(19) United States
(12) Patent Application Publication
(10) Pub. No.: US 2015/0007357 A1
(43) Pub. Date: Jan. 1, 2015

(54) Use Of Humanized Mice To Determine Toxicity

(71) Applicant: Massachusetts Institute Of Technology, Cambridge, MA (US)

(72) Inventors: Salim Bouguermouh, Singapore (SG); Maroun Khoury, Santiago (CL); Qingfeng Chen, Singapore (SG); Jianzhu Chen, Lexington, MA (US)

(21) Appl. No.: 14/362,774

(22) PCT Filed: Dec. 5, 2012
(86) PCT No.: PCT/US2012/067983

§ 371 Date: Jun. 4, 2014

Related U.S. Application Data
(60) Provisional application No. 61/567,466, filed on Dec. 6, 2011.

(51) Int. Cl. A61K 49/00

(52) U.S. Cl. CPC A61K 49/0008
USPC 800/3

(57) ABSTRACT

The invention is directed to a method of determining whether an agent causes immune toxicity in a human comprising administering the agent to a non-human mammal that has been engrafted with human hematopoietic stem cells (HSCs) and administered one or more human cytokines; and determining whether the agent causes immune toxicity in the non-human mammal. If the agent causes immune toxicity in the non-human mammal then the agent causes toxicity in a human. The invention is also directed to a method of determining whether administration of an agent causes cytokine release syndrome in an individual in need thereof comprising administering the agent to a non-human mammal that has been engrafted with HSCs and administered one or more human cytokines; and determining whether the agent causes cytokine release syndrome in the non-human mammal. If the agent causes cytokine release syndrome in the non-human mammal then the agent will cause cytokine release syndrome in the human.

### RELATED APPLICATION(S)

This application claims the benefit of U.S. Provisional Application No. 61/567,466, filed on Dec. 6, 2011.

The entire teachings of the above application are incorporated herein by reference.

### BACKGROUND OF THE INVENTION

A significant gap exists between pre-clinical and clinical testing, as close-to-human models are often unable to accurately predict many adverse effects. In a phase-I clinical trial, the administration of TGN1412, a humanized superagonistic CD28 monoclonal antibody (IgG4), developed for the treatment of autoimmune disease, led to catastrophic events associated with “cytokine storm” symptoms. The necessity for a model that can accurately predict such adverse effects is immense. The immunodeficient NOD-scid IL2rγnull mice with an enhanced engraftment property of human immune cells present an appealing model. However, their widespread use has been limited due to the weak human immune responses observed in the current model.

Thus, a need exists for improved models that can accurately predict adverse effects of agents (e.g., therapeutic agents) on the immune system in humans.

### SUMMARY OF THE INVENTION

The immune responses of a humanized mouse model are improved by injecting human hematopoietic stem cells and human cytokines (e.g., human IL-15 and Flt-3L cytokines) into a mouse. Shown herein is that the cytokine-treated humanized (CTH) mouse model was capable of predicting, with a high degree of fidelity, adverse effects of four distinct monoclonal antibodies used in clinics or in preclinical trial.

Accordingly, in one aspect, the invention is directed to a method of determining whether an (one or more) agent causes immune toxicity in a human. The method comprises administering the agent to a non-human mammal that has been engrafted with human hematopoietic stem cells (HSCs) and administered one or more human cytokines; and determining whether the agent causes immune toxicity in the non-human mammal, wherein if the agent causes immune toxicity in the non-human mammal then the agent causes toxicity in a human.

In another aspect, the invention is directed to a method of determining whether administration of an (one or more) agent causes cytokine release syndrome in an individual (e.g., human) in need thereof. The method comprises administering the agent to a non-human mammal that has been engrafted with human hematopoietic stem cells (HSCs) and administered one or more human cytokines; and determining whether the agent causes cytokine release syndrome in the non-human mammal, wherein if the agent causes cytokine release syndrome in the non-human mammal then the agent will cause cytokine release syndrome in the human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: The injections of TGN 1412 induced a large panel of systemic pro-inflammatory cytokine production. The cytokine production was measured in the sera of TGN 1412-IgG4, a humanized version of the mouse anti-human CD28 antibody 5.11A1 (isotype: human IgG4/Kappa) also referred to herein as TGN1412, and TGN1412-AA, a humanized FcR-non-binding version of the mouse anti-human CD28 antibody 5.11A1 (Isotype: human IgG4/Kappa), treated groups at 48 hours before treatment, 2 and 24 hours post-injection. The values were also compared to the saline-injected control group. The amount of human interleukin-2 (hIL-2), hIL-6, hIL-8, hIL-1β, hIL-4, hIFN-γ and hTNF-α were determined by BD FACSArray analysis. Each symbol represents one mouse. The same symbol represents the same mouse at different time points. The n values indicate the number of mice used in each group, from two independent experiments.

FIGS. 2A-2B: A change in T and NK cell percentages and absolute numbers was noted following TGN1412 treatment. Mice were bled at the indicated time points and PBMCs were analyzed for human CD45 (hCD45). (2A) Comparison of percentages of CD3⁺, CD14⁺, CD19⁺, CD56⁺, gated on human CD45 cells and CD4⁺ and CD8⁺; gated on CD45⁺CD3⁺ cells from PBMCs of mice following flow cytometry analysis is represented. (2B) The absolute numbers of the different cell lineages were calculated following a correlation between the number of viable cells counted using the hematocytometer and the percentage of the corresponding cell population obtained from the flow cytometry analysis.

FIG. 3: The injections of TGN1412-1gG4 to humanized mice not treated with IL-15 and FLT3L cytokines was not followed by an increase in cytokine production. The cytokine production was measured in the sera of TGN1412-IgG4 treated groups at 48 hours before treatment, 2 and 24 hours post-injection. The values were also compared to the saline-injected control group. The amount of hIL-2, hIL-6, hIL-8, hIL-1b, hIL-4, hIFN-γ and hTNF-α were determined by BD FACSArray analysis. Each symbol represents one mouse. The same symbol represents the same mouse at different time points. The n values indicate the number of mice used in each group, from a single experiment.

FIGS. 4A-4B: A change in T, B and NK cell percentages and absolute numbers was noted following TGN1412 treatment of humanized mice not treated with IL-15 and FLT3L cytokines. Mice were bled at the indicated time points and PBMCs were analyzed for human CD45 (hCD45). (4A) Comparison of percentages of CD3⁺, CD14⁺CD19⁺, CD56⁺, gated on human CD45 cells and CD4⁺ and CD8⁺ gated on CD45⁺CD3⁺ cells from PBMCs of mice following flow cytometry analysis is represented. (4B) The absolute numbers of the different cell lineages were calculated following a correlation between the number of viable cells counted using the hematocytometer and the percentage of the corresponding cell population obtained from the flow cytometry analysis.

FIG. 5: The injection of OKT3 induced a large panel of systemic pro-inflammatory cytokine production. The cytokine production was measured in the sera of OKT3 treated group at 48 hours before treatment, 2 and 24 hours post-injection. The values were also compared to the saline-injected control group. The amount of hIL-2, hIL-6, hIL-8, hIL-1β, hIL4, hlFN-γ and hTNF-α were determined via BD FACSArray. Each symbol represents one mouse. The same symbol represents the same mouse at different time points. The n values indicate the number of mice used in each group, from three independent experiments.

FIGS. 6A-6B: A change in several cell percentages and absolute numbers was noted following OKT3 treatment. Mice were bled at the indicated time points and PBMCs were analyzed for human CD45 (hCD45) as mentioned in FIG. 1. (6A) Comparison of percentages of CD3⁺CD14⁺CD19⁺CD56⁺, gated on human CD45 cells from PBMCs of mice following flow cytometry analysis is represented. (6B) The absolute numbers of the different cell lineages is presented.

FIG. 7: The injections of Alemtuzumab showed a slightly increase of pro inflammatory cytokines. The cytokines production was measured in the sera of Alemtuzumab treated group at 48 hours before treatment, 2 and 24 hours post-injection. The values were also compared to the saline-injected control group. The n values indicate the number of mice used in each group, from two independent experiments.

FIGS. 8A-8B: The injection of Alemtuzumab was associated with a drastic decrease of lymphocytes, NK cells and monocytes. Mice were bled at the indicated time points and PBMCs were analyzed for human CD45 (hCD45) as mentioned in FIG. 1. (8A) Comparison of percentages of CD3⁺, CD14⁺, CD19⁺ and CD56⁺, gated on human CD45⁺ cells from PBMCs of mice following flow cytometry analysis is represented. (8B) The absolute numbers of the different cell lineages is presented.

FIG. 9: The injections of Rituximab did not show any noticeable effect on the production of systemic pro-inflammatory cytokines. The cytokines production was measured in the sera of Rituximab treated group at 48 hours before treatment, 2 and 24 hours post-injection. The values were also compared to the saline-injected control group. The amount of the different measured cytokine remained unchanged. The n values indicate the number of mice used in each group, from two independent experiments.

FIGS. 10A-10B Among all the different cell lineages analysed, only B cells showed a change in the percentage and total numbers. Mice were bled at the indicated time points and PBMCs were analyzed for human CD45 (hCD45) as mentioned in FIG. 1. (10A) Comparison of percentages of CD3⁺, CD14⁺, CD19⁺; and CD56⁺, gated on human CD45⁺ cells from PBMCs of mice following flow cytometry analysis is represented. (10B) The absolute numbers of the different cell lineages is presented.

FIGS. 11A-11D Detection of liver enzymes after administration of TGN1412-IgG4, TGN1412-AA, OKT3, Alemtuzumab and Rituximab in cytokine treated (11A, 11B) and none treated mice (11C, 11D). (11A, 11B) The administration of TGN1412-IgG4 was associated with an elevation of the Aspartate and Alanine aminotransferase (AST and ALT) levels at 24 hours post-treatment. The injection of TGN1412-AA was only associated with an elevation of ALT. The administration of OKT3, Alemtuzumab and Rituximab did not induce a significant increase in any of the tested liver enzymes. (11C, 11D) The administration of TGN1412-IgG4 did not induce an augmentation in any of the tested liver enzymes.

FIGS. 12A-12B: Cells isolated from the spleen of 2 control humanized mice were stained for human antibodies and analyzed by flow cytometry. A representative plot showing the expression of CD28 on (12A) CD45⁺CD3⁺CD4⁺ and CD45⁺CD3⁺CD4⁺ human cells and (12B) naive, effector and memory CD4⁺ cells.

FIG. 13: Total cells isolated from the spleen of control, TGN1214-IgG4 and TGN1214-AA were counted and stained for the different human antibodies.

FIG. 14: Total cells isolated from the spleen of control, TGN1214-IgG4 were counted and stained for the different human antibodies.

FIG. 15: The administration of TGN1412 and OKT3 to the mice treated with human IL-15 and FLT3L human cytokines plasmid induce a large panel of systemic pro-inflammatory cytokine production although the injections of Alemtuzumab show a slightly increase of hIL-6 and hIL-8 cytokines. The injections of Rituximab do not show any noticeable effect on the production of pro-inflammatory cytokines. The cytokine production was measured in the sera of TGN1412-IgG4, TGN1412-AA, OKT3, Alemtuzumab and Rituximab treated groups at 48 hours before treatment, 2 and 24 hours post-injection. The values where also compared to the saline-injected control group. The amount of hIL-2, hIFN-γ, hIL-6, hIL-8, hTNF-α, hIL-1β and hIL-4 were determined by BD FACSArray analysis. Each symbol represents one mouse. The n values indicate the number of mice used in each group, from at least three independent experiments. Statistical analysis by Kruskal-Wallis Test (Nonparametric ANOVA): *P≦0.05; **P≦0.005; ***P≦0.0001.

FIGS. 16A-16H: A change in T, NK and B cells cell percentages and absolute numbers is noted following TGN1412, OKT3 Alemtuzumab and Rituximab treatment of the mice injected with IL-15 and FLT3L human cytokines plasmid. Mice were bled at the indicated time points and PBMCs were analyzed for human CD45 (hCD45) and mouse CD45 (mCD45). (16A, 16B and 16C) The absolute numbers of the different cell lineages were calculated following a correlation between the numbers of viable cells counted using the Hematocytometer and the percentage of the corresponding cell population obtained from the flow cytometry analysis. (16D) Comparison of percentages of CD3+, CD14+, CD19+, CD56+ gated on human CD45, (16E and 16F) hCD45 and mCD45 gated on live cells and (16G) CD4+ and CD8+ gated on CD45+CD3+ cells from PBMCs of mice following flow cytometry analysis is represented. (16H) Comparison of the level of human CD45+ cells reconstitution. Statistical analysis by Kruskal-Wallis Test (Nonparametric ANOVA): *P≦0.05; **P≦0.005; ***P≦0.0001.

FIG. 17: The injections of TGN1412-IgG4 to humanized mice not treated with IL-15 and FLT3L human cytokines plasmid is not followed by systemic cytokine release. The cytokine production was measured in the sera of TGN1412-IgG4 treated groups at 48 hours before treatment, 2 and 24 hours post-injection. The values where also compared to the saline-injected control group. The amount of hIL-2, hIFN-γ hIL-6, hIL-8, hTNF-α, hIL-1β and hIL-4 were determined by BD FACSArray analysis. Each symbol represents one mouse. The same symbol represents the same mouse at different time points. The n values indicate the number of mice used in each group, from a single experiment.

FIGS. 18A-18H: A change in T, B and NK cell percentages and absolute numbers is noted following TGN1412 treatment of humanized mice not treated with IL-15 and FLT3L cytokines. Mice were bled at the indicated time points and PBMCs were analyzed for human CD45 (hCD45). (18A, 18B and 18C) The absolute numbers of the different cell lineages were calculated as previously. (18D) Comparison of percentages of CD3+, CD14+, CD19+, CD56+ gated on human CD45, (18E and 18F) hCD45 and mCD45 gated on live cells and (18G) CD4+ and CD8+ gated on CD45+CD3+ cells from PBMCs of mice following flow cytometry analysis is represented. (18H) Comparison of the level of human CD45+ cells reconstitution. Statistical analysis by Kruskal-Wallis Test (Nonparametric ANOVA); *P≦0.05; **P≦0.005.

FIG. 19: The injections of TGN1412 and OKT3 to the mice treated with M-CSF cytokines increased the production of systemic IL-6 and IL-8. The cytokine production was measured in the sera of TGN1412-IgG4 and OKT3 treated groups at 48 hours before treatment, 2 and 24 hours post-injection. The values where also compared to the saline-injected control group. The amount human cytokines were determined as described previously. Each symbol represents one mouse. The n values indicate the number of mice used in each group, from at least three independent experiments. Statistical analysis by Kruskal-Wallis Test (Nonparametric ANOVA) and Paired t test: *P≦0.05; **P≦0.005; ***P≦0.0001.

FIGS. 20A-20H: A change in T, NK and B cells cell percentages and absolute numbers is noted following TGN1412-IgG and OKT3 treatment of the mice injected with M-CSF human cytokine plasmid. Mice were bled at the indicated time points and PBMCs were analyzed for human CD45 (hCD45) and mouse CD45 (mCD45). (20A, 20B and 20C) The absolute numbers of the different cell lineages were calculated as previously. (20D) Comparison of percentages of CD3+, CD14+, CD19+, CD56+ gated on human CD45, (20E and 20F) hCD45 and mCD45 gated on live cells and (20G) CD4+ and CD8+ gated on CD45+CD3+ cells from PBMCs of mice following flow cytometry analysis is represented. (20H) Comparison of the level of human CD45+ cells reconstitution. Statistical analysis by Kruskal-Wallis Test (Nonparametric ANOVA): *P≦0.05; **P≦0.005.

FIG. 21: The injections of TGN1412 and OKT3 to the mice treated with IL-15 and FLT3L or M-CSF human cytokines plasmid induce a slightly increase of mouse mIL-6 but not mIL-2 or mIFN-γ. The cytokine production was measured in the sera at the same time point use for human cytokines. The amount of mIL-2, mIFN-γ and mIL-6 were determined by BD FACSArray analysis. Each symbol represents one mouse. The n values indicate the number of mice used in each group, from at least tow independent experiments. Statistical analysis by Kruskal-Wallis Test (Nonparametric ANOVA): *P≦0.05.

FIGS. 22A-22D: Detection of liver enzymes after administration of TGN1412-IgG4, TGN1412-AA, OKT3, Alemtuzumab and Rituximab in cytokine treated (22A, 22B) and none treated mice (22C, 22D). (22A, 22B) The administration of TGN1412-IgG4 is associated with an elevation of the Aspartate and Alanine aminotransferase (AST and ALT) levels at 24 hours post-treatment. The injection of TGN1412-AA was only associated with an elevation of ALT. The administration of OKT3, Alemtuzumab and Rituximab did not induce a significatively increase in any of the tested liver enzymes. (22C, 22D) The administration of TGN1412-IgG4 did not induce an augmentation in any of the tested liver enzymes. Statistical analysis by Kruskal-Wallis Test (Nonparametric ANOVA): *P≦0.05; **P≦0.005.

FIGS. 23A-23B: Cells isolated from the spleen of 2 control humanized mice were stained for human antibodies and analyzed by flow cytometry. A representative plot showing the expression of CD28 on (23A) CD45+CD3+CD8+ and CD45+CD3+CD4+ human cells and (23B) Naïve, effector and memory CD4+ cells.

FIG. 24: Total cells isolated from the spleen of control, TGN1214-IgG4 and TGN1214-AA were counted and stained for the different human antibodies.

FIG. 25: Total cells isolated from the spleen of control, TGN1214-IgG4 were counted and stained for the different human antibodies.

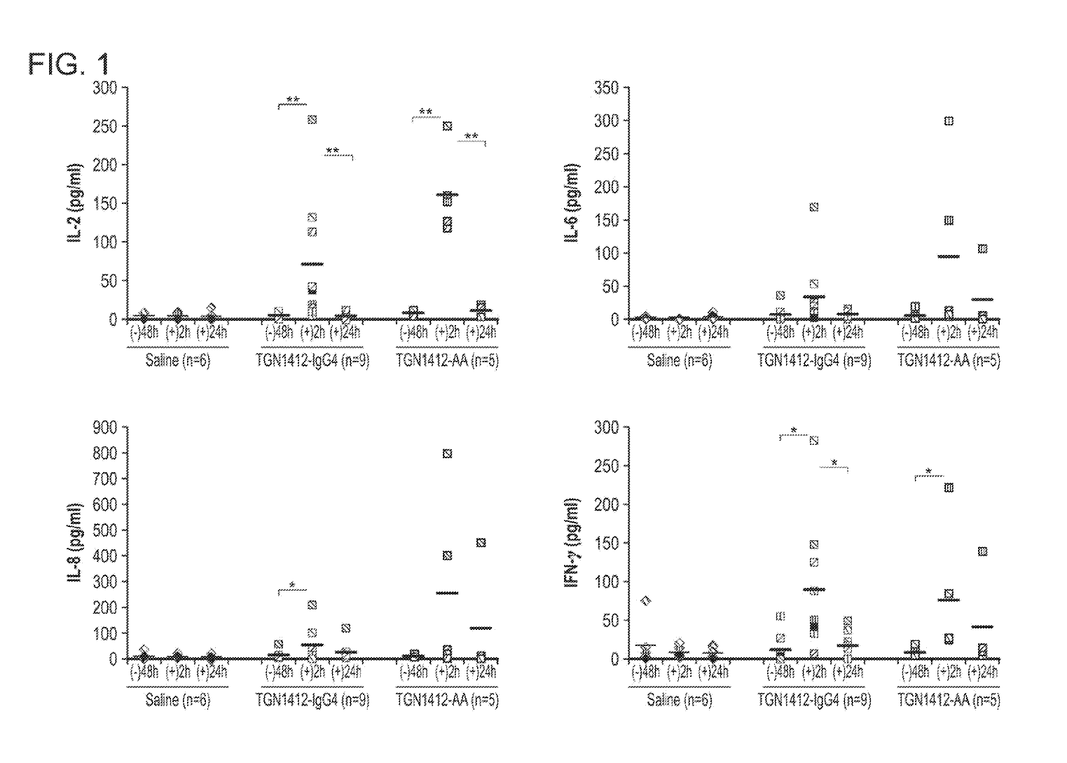

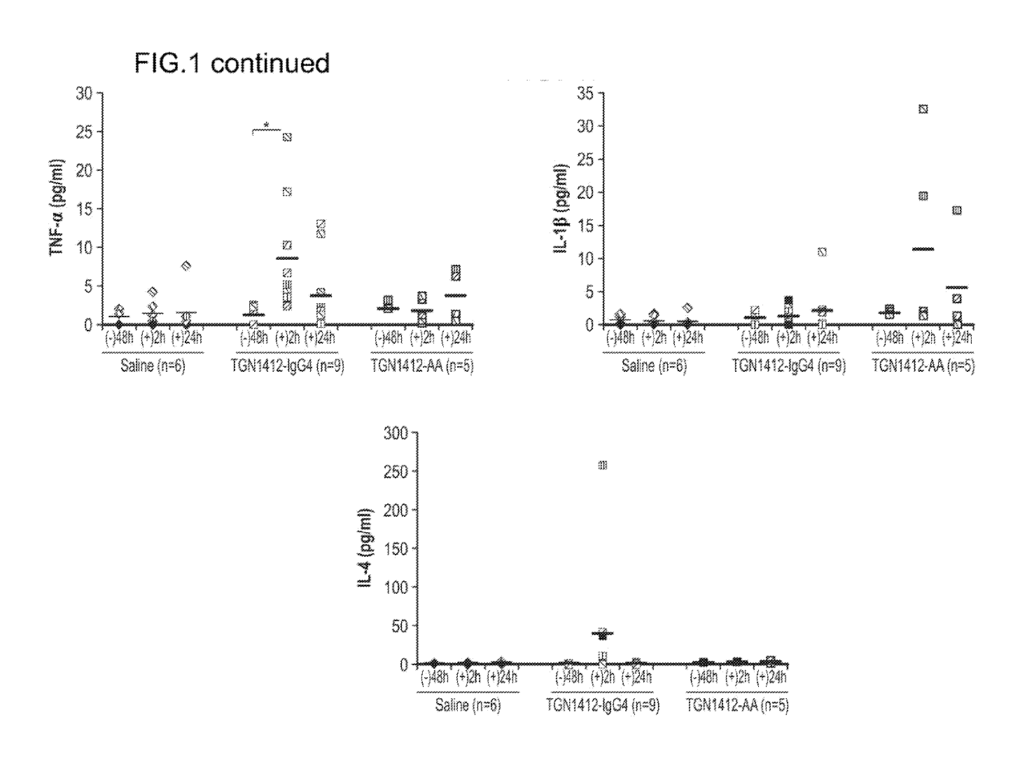

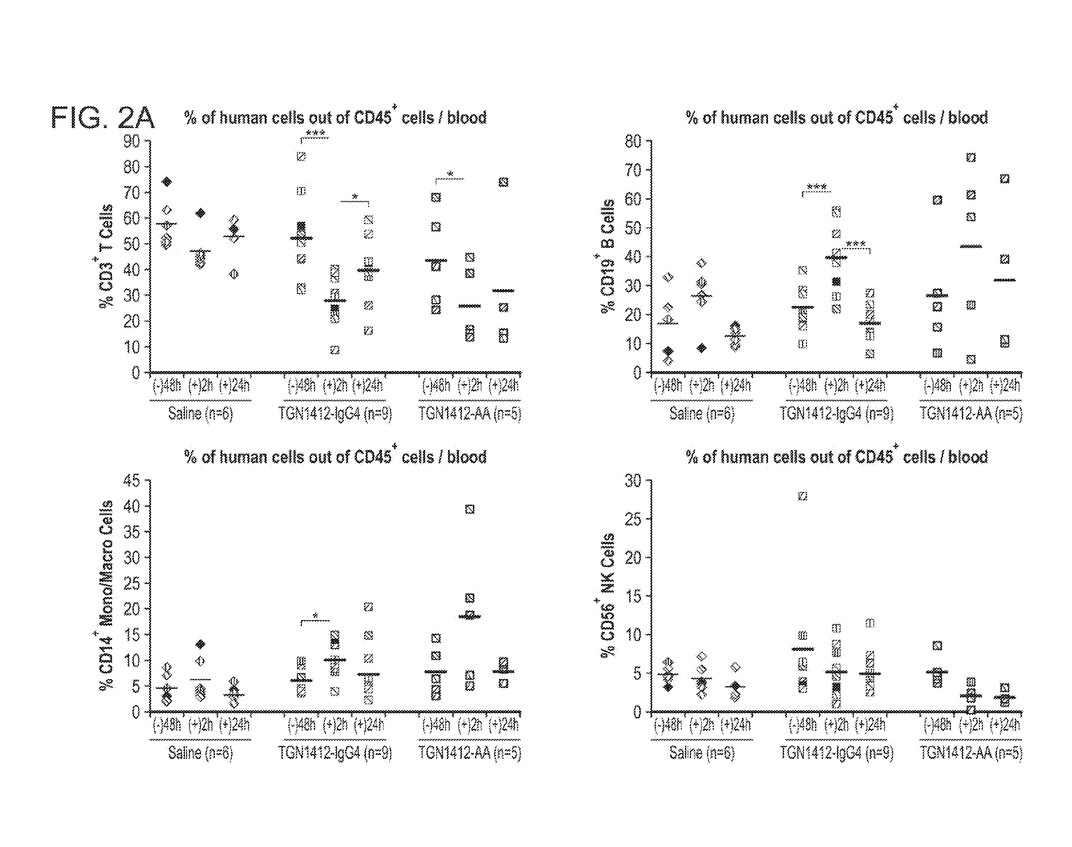

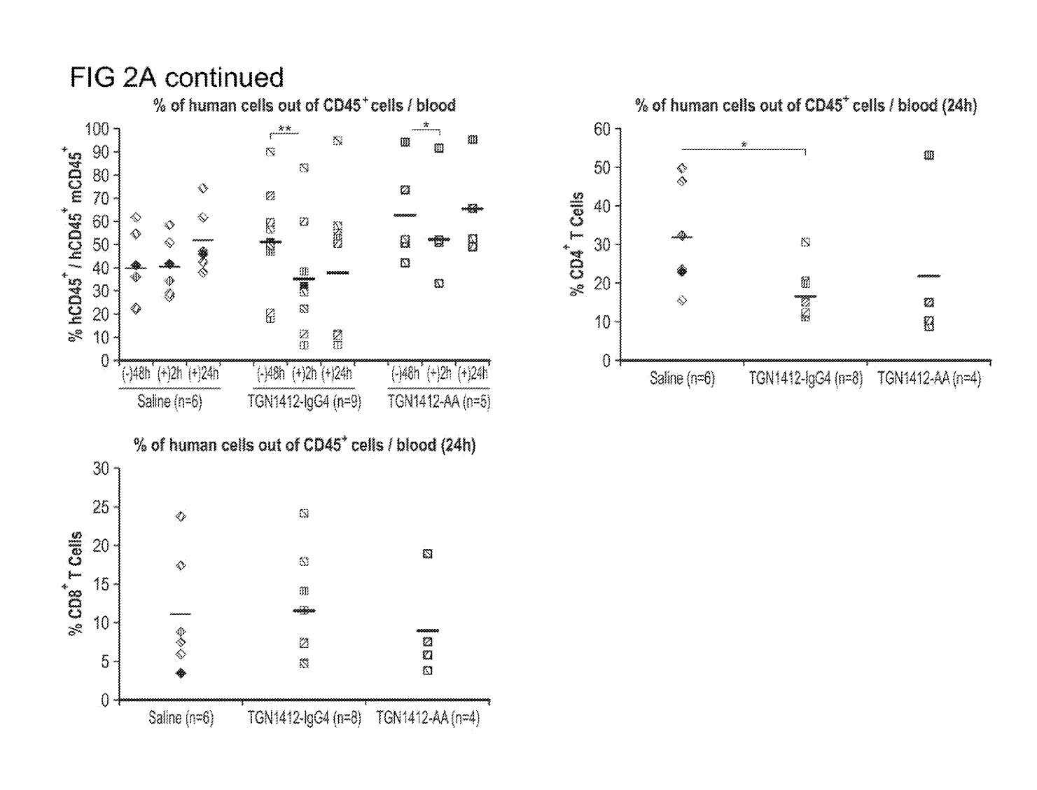

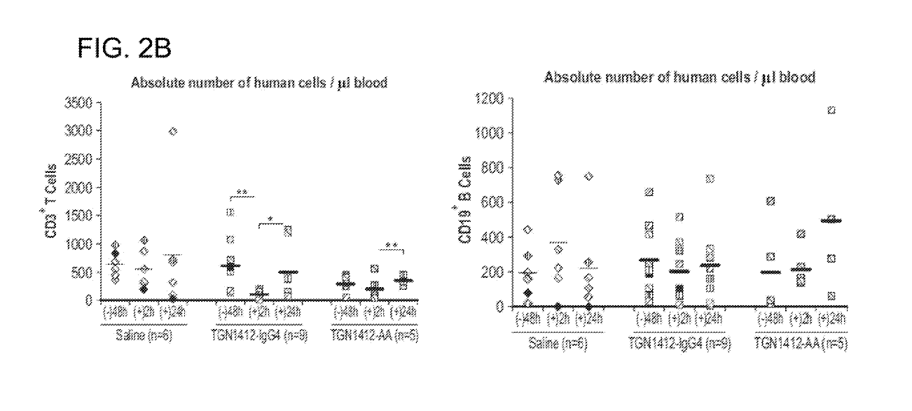

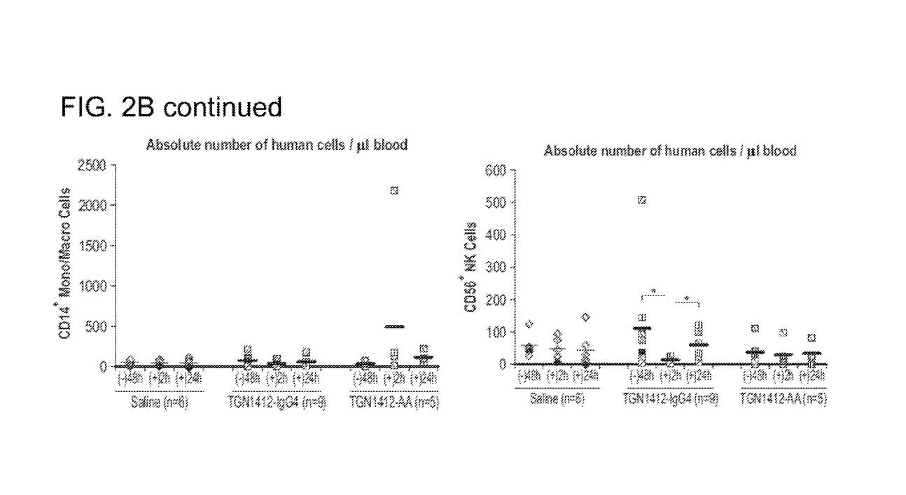

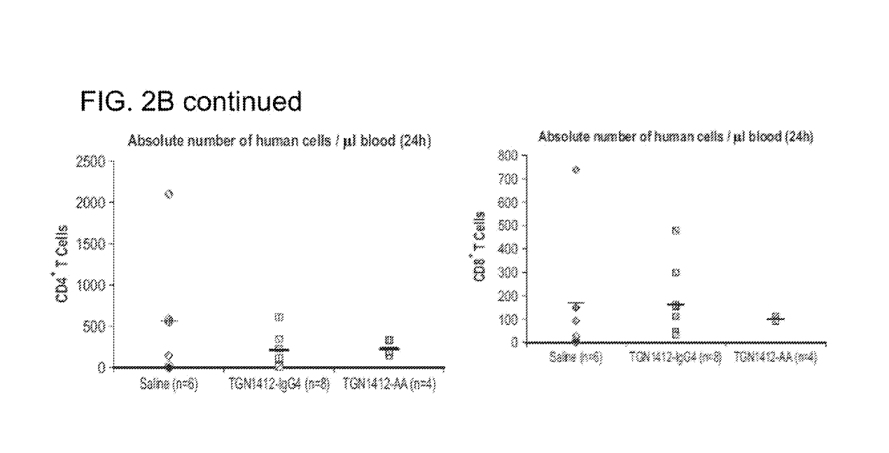

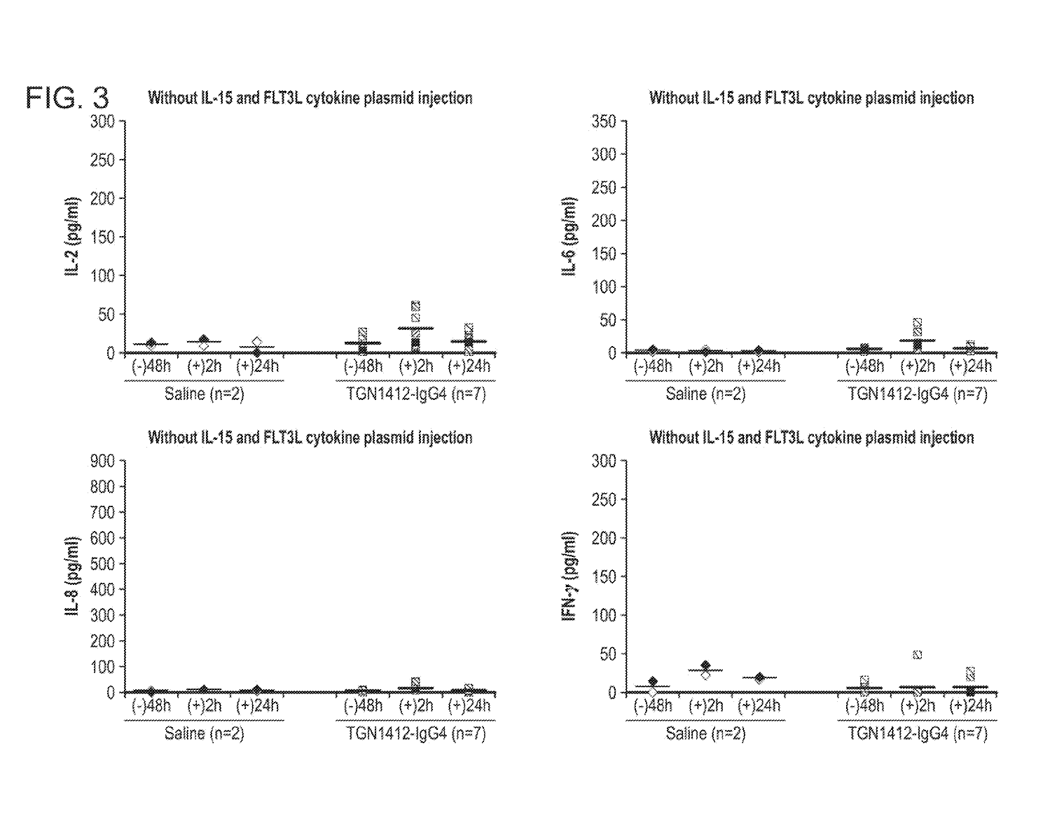

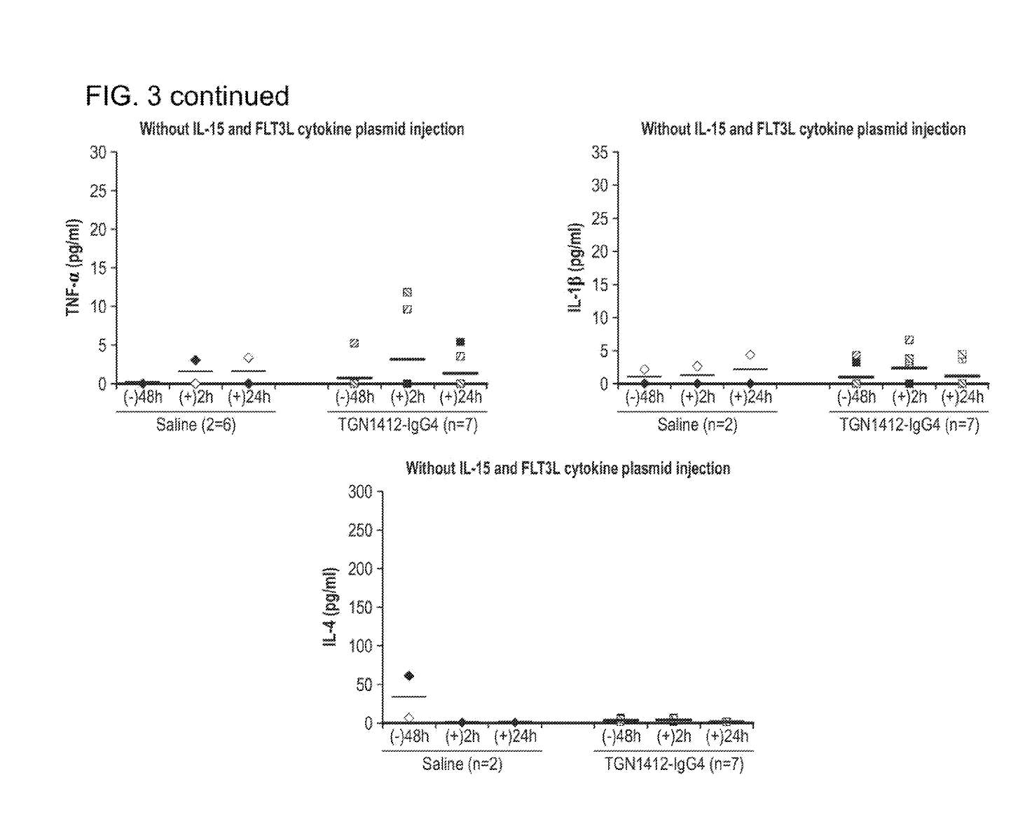

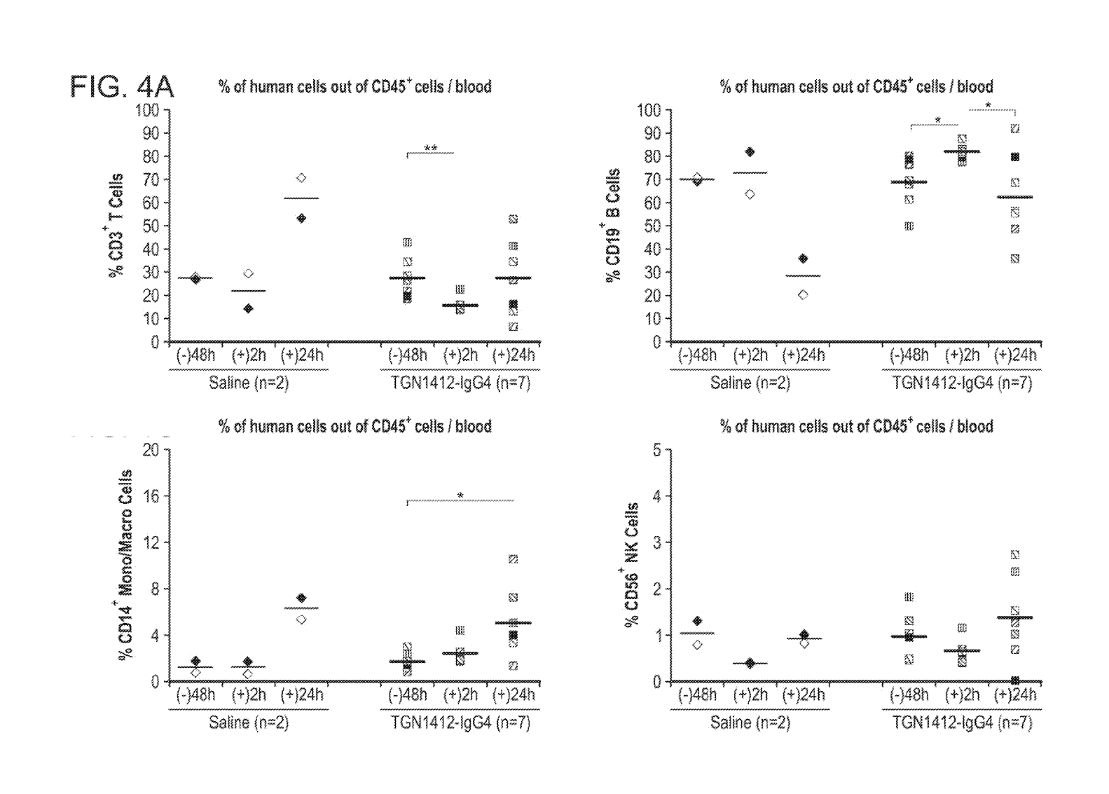

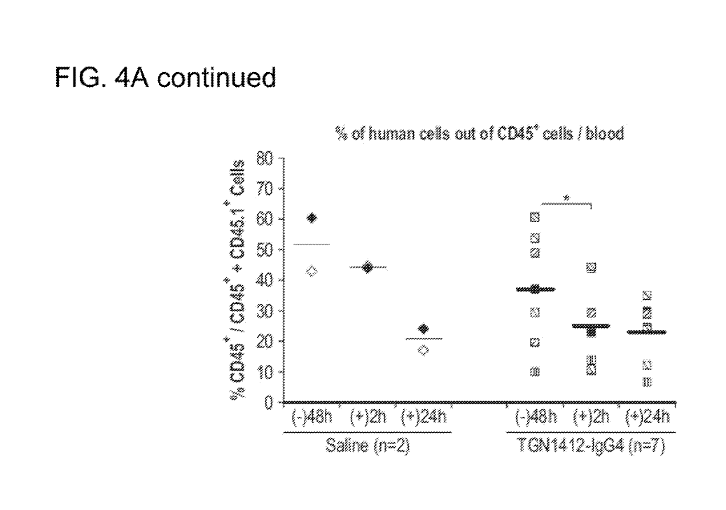

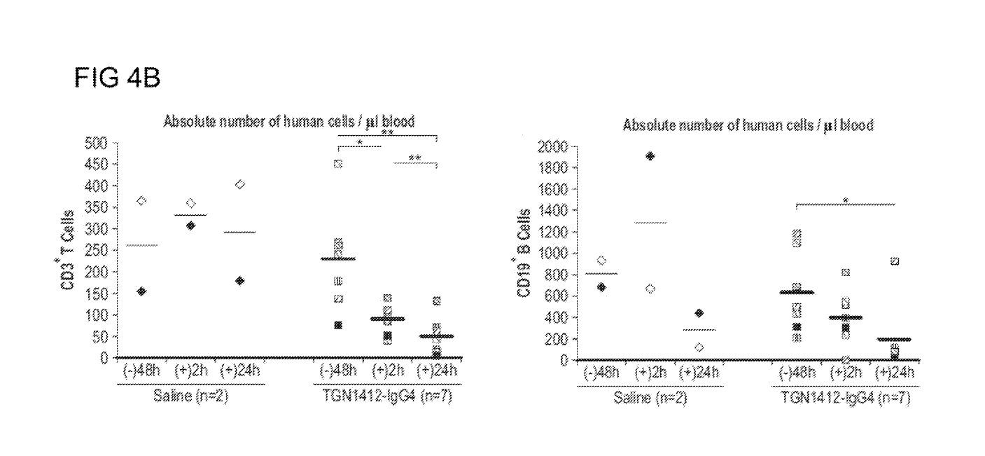

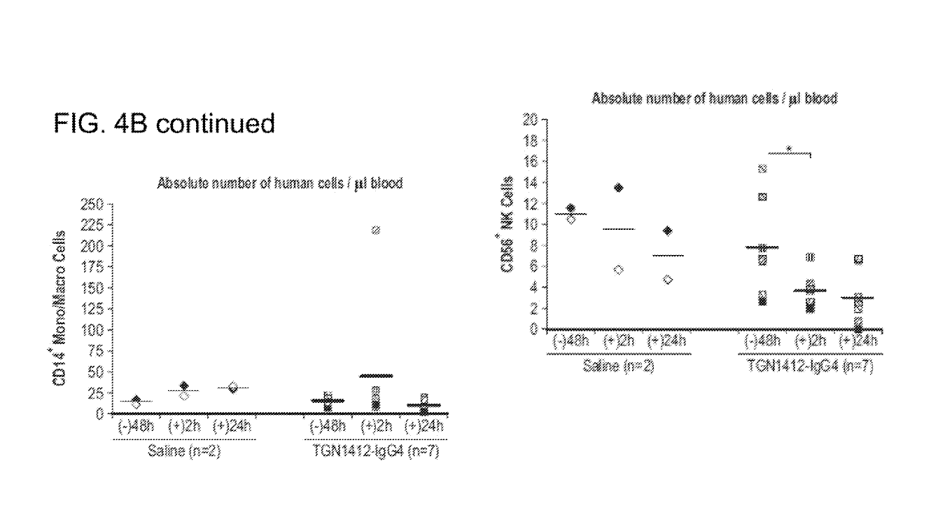

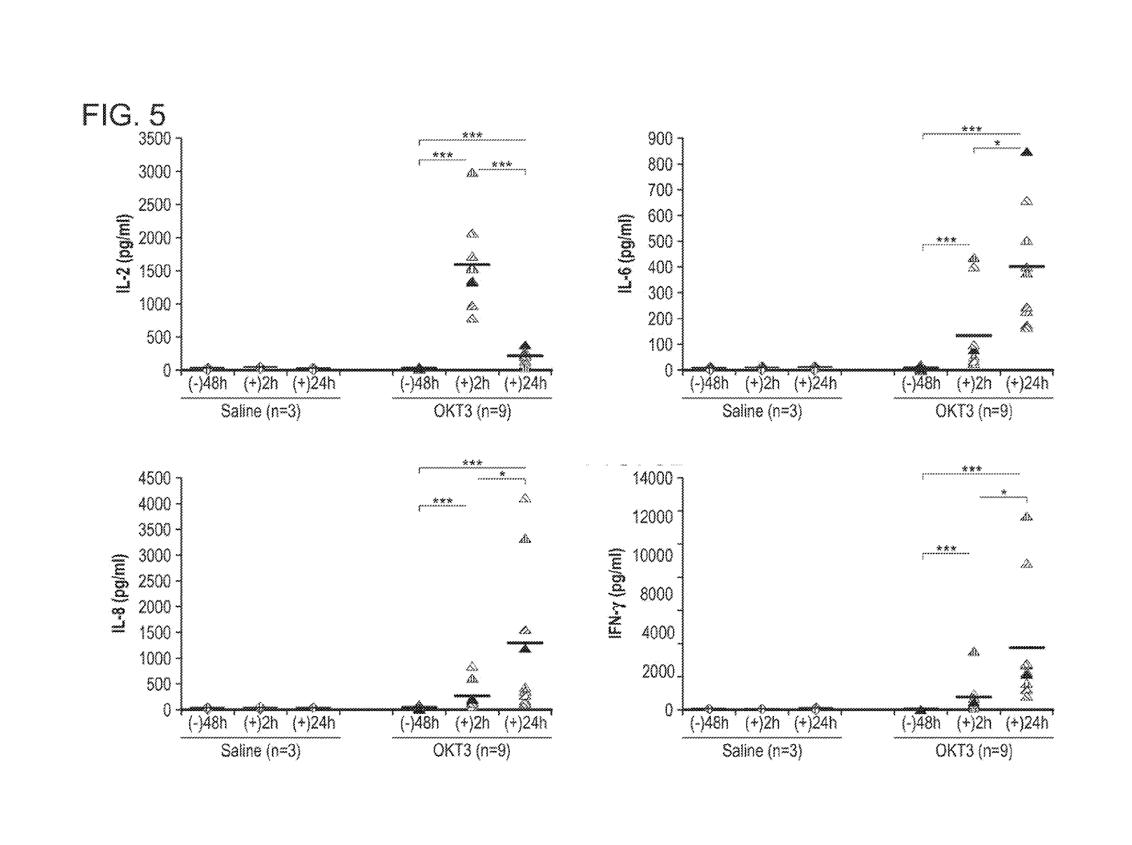

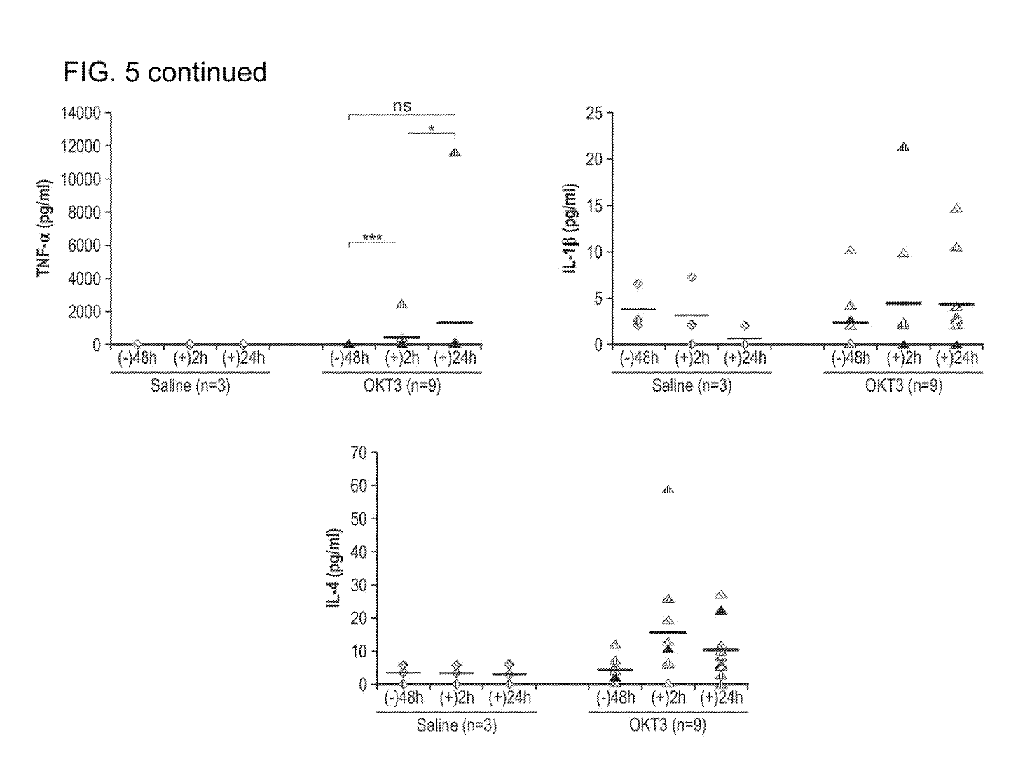

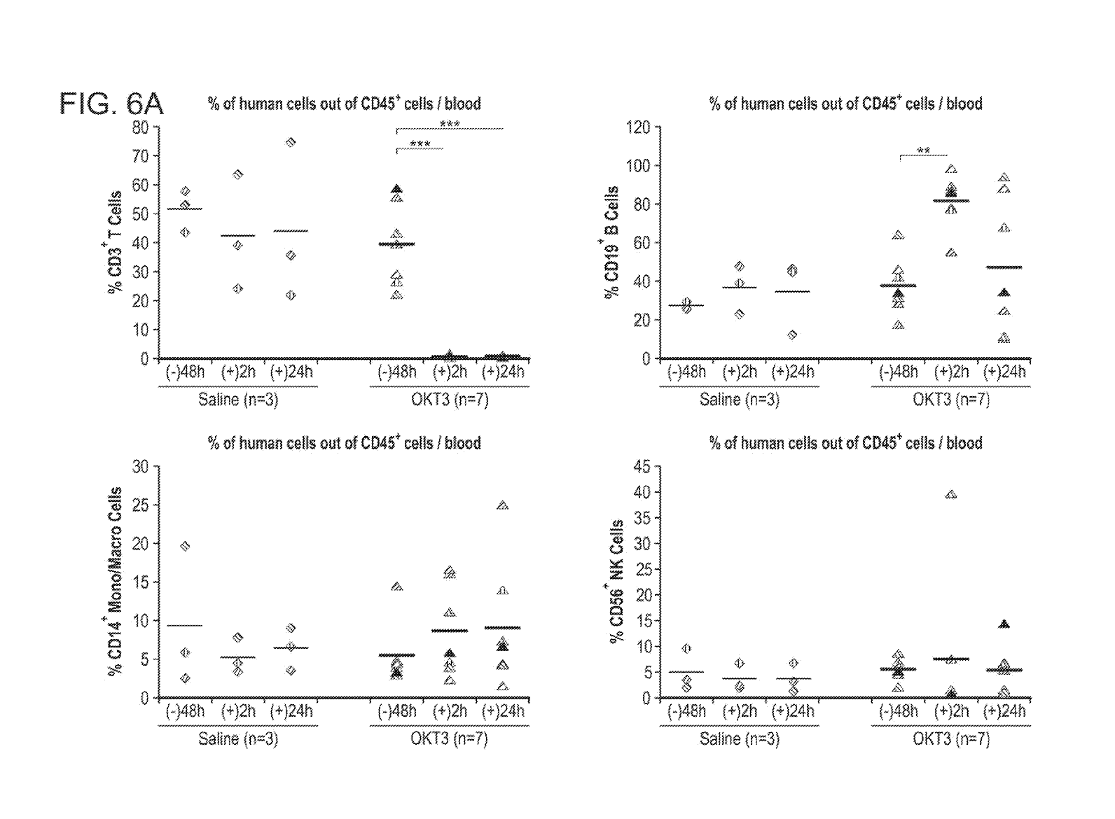

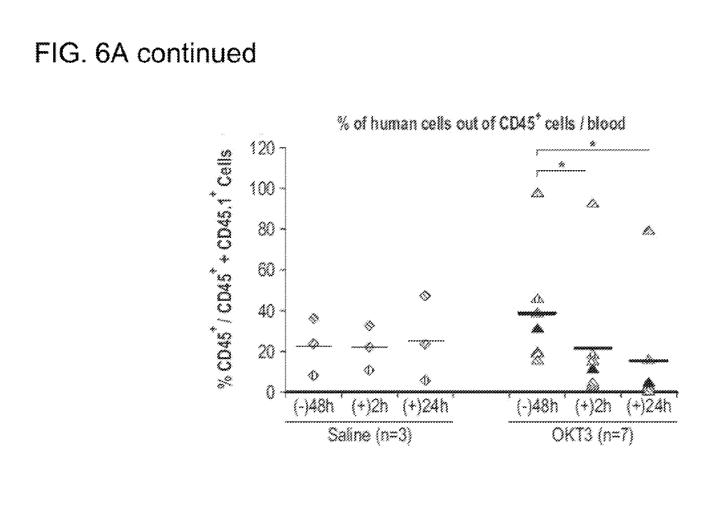

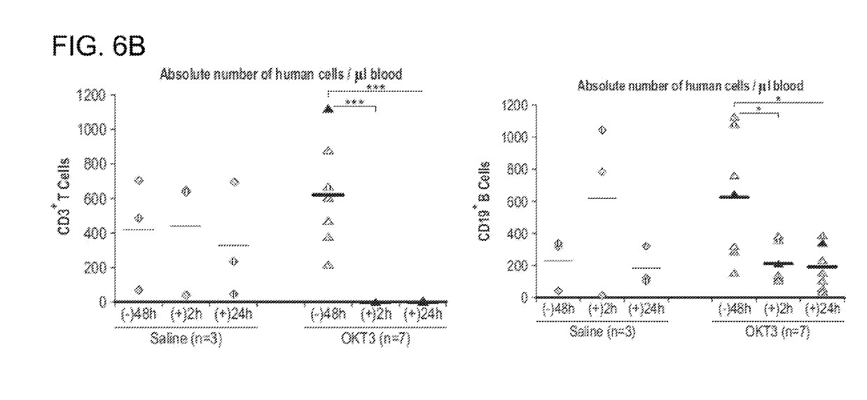

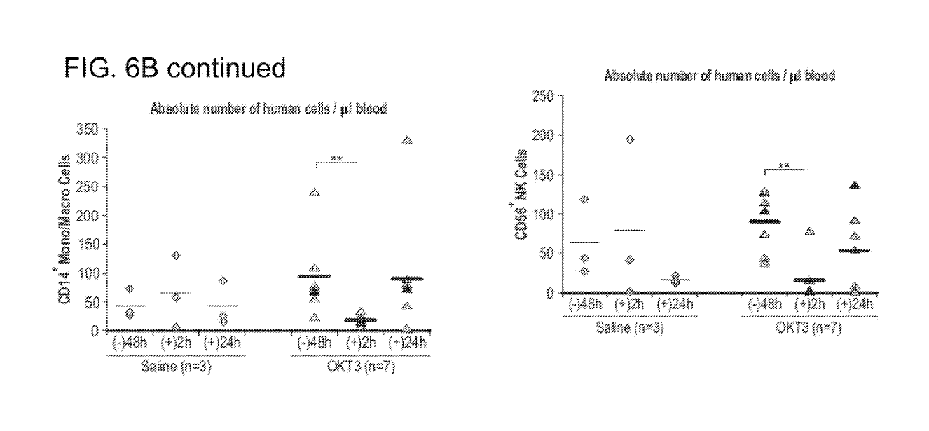

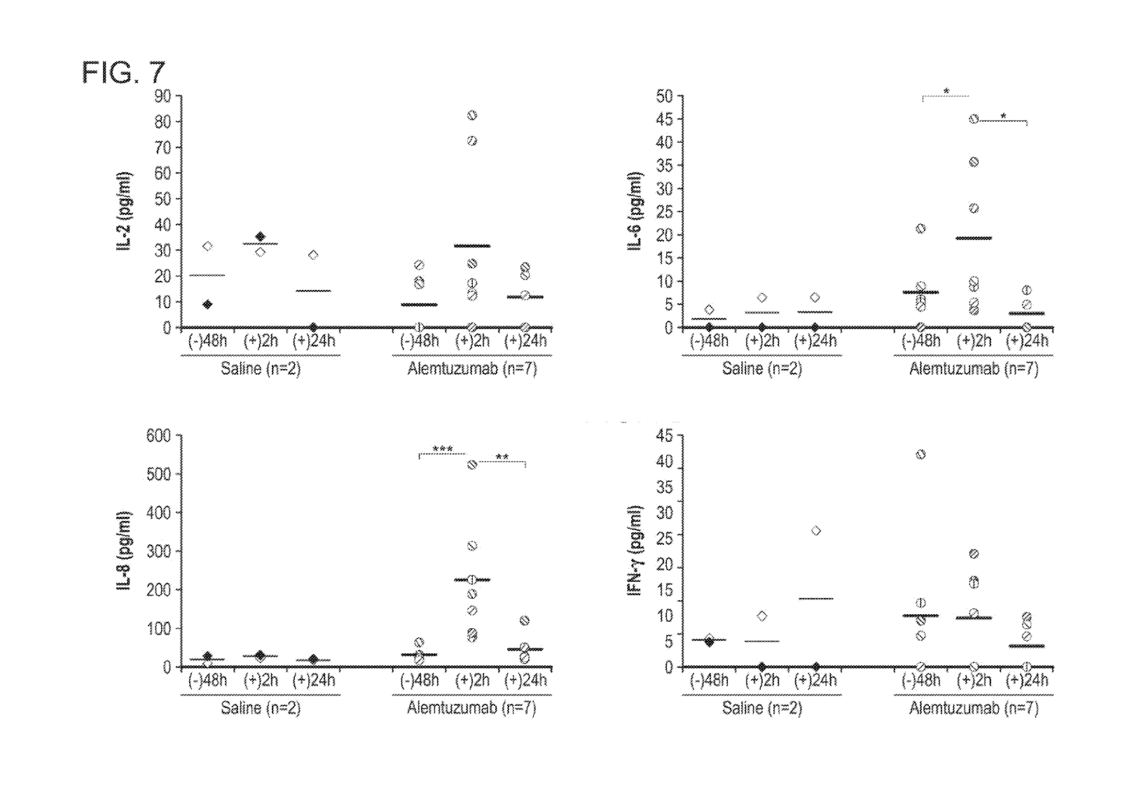

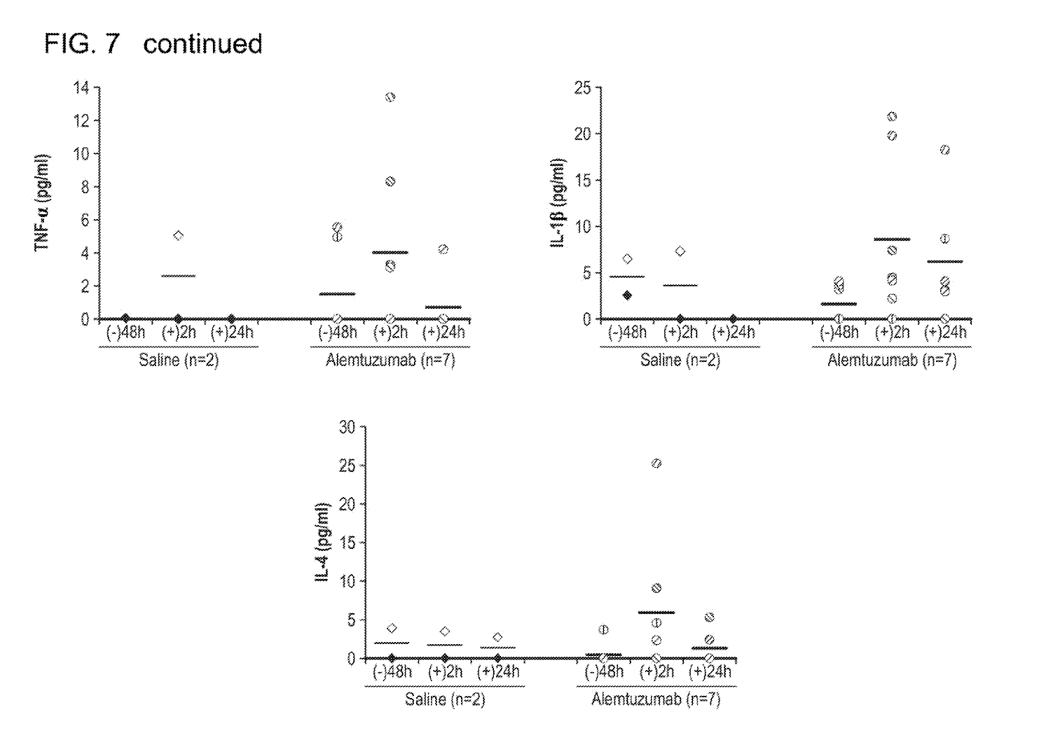

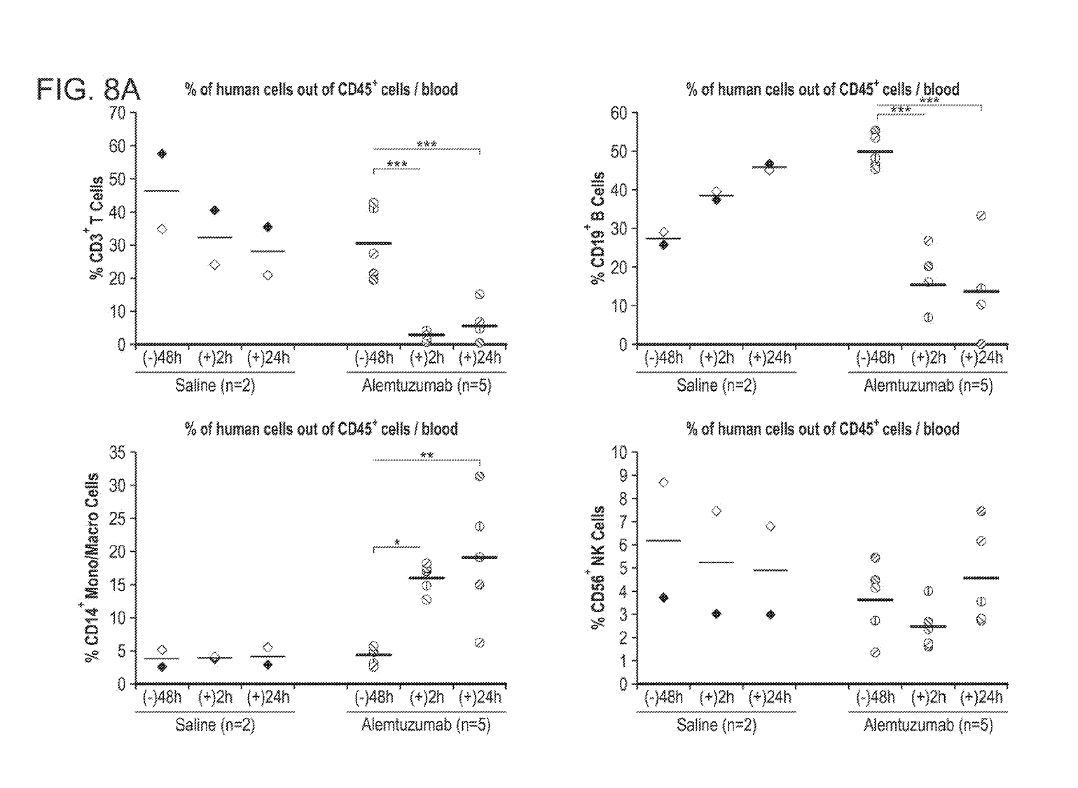

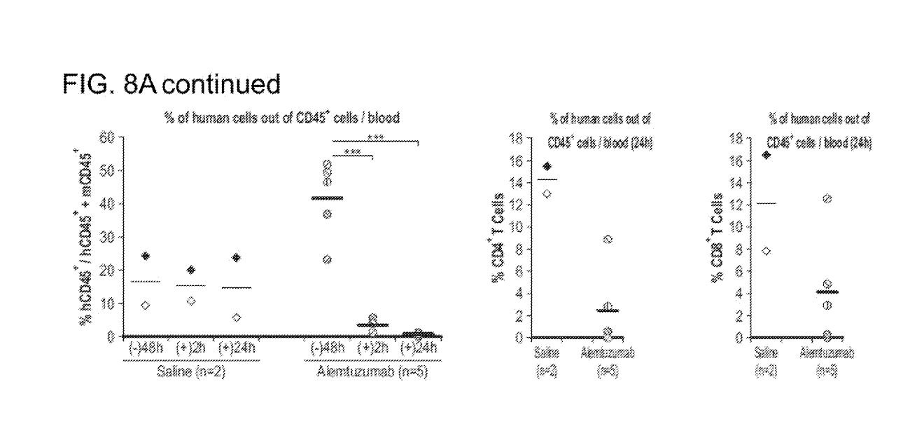

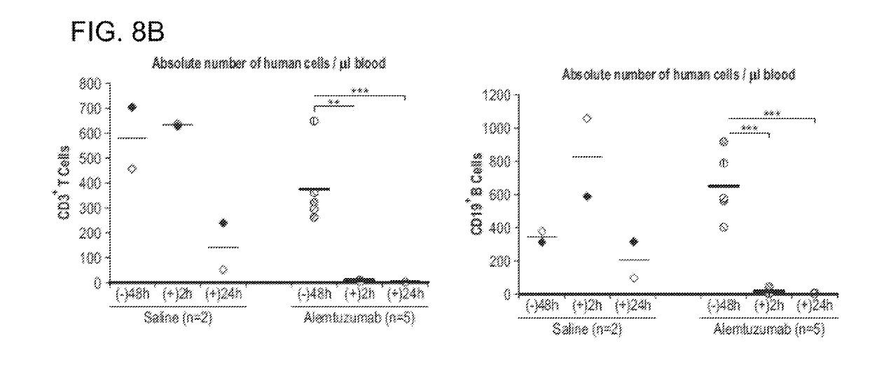

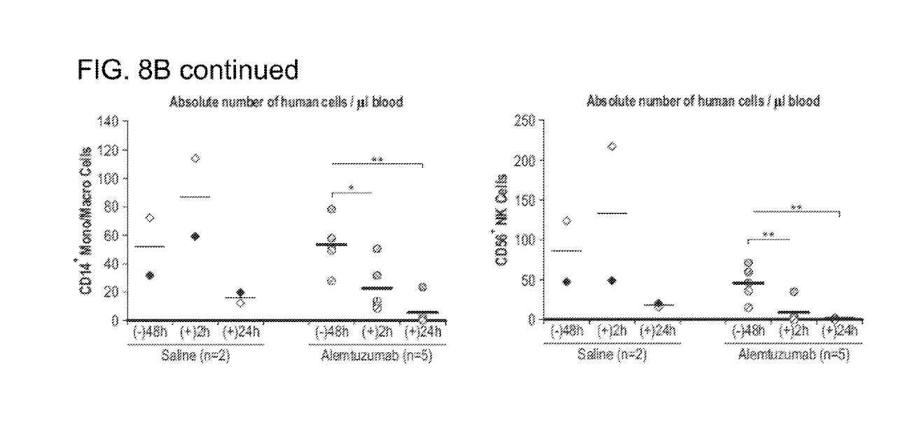

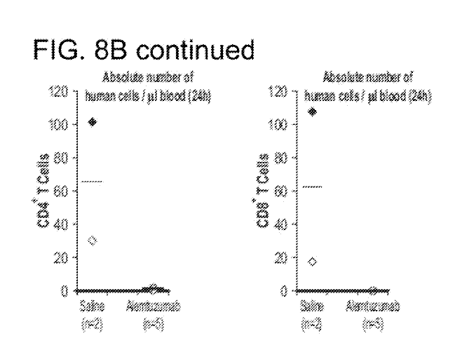

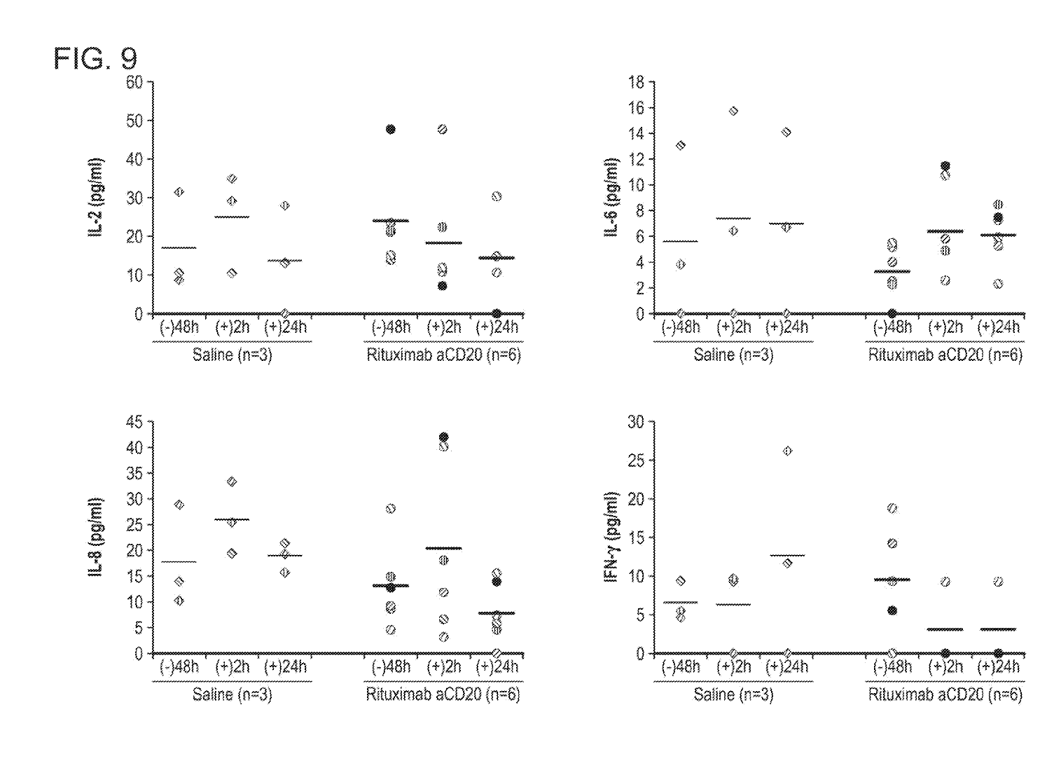

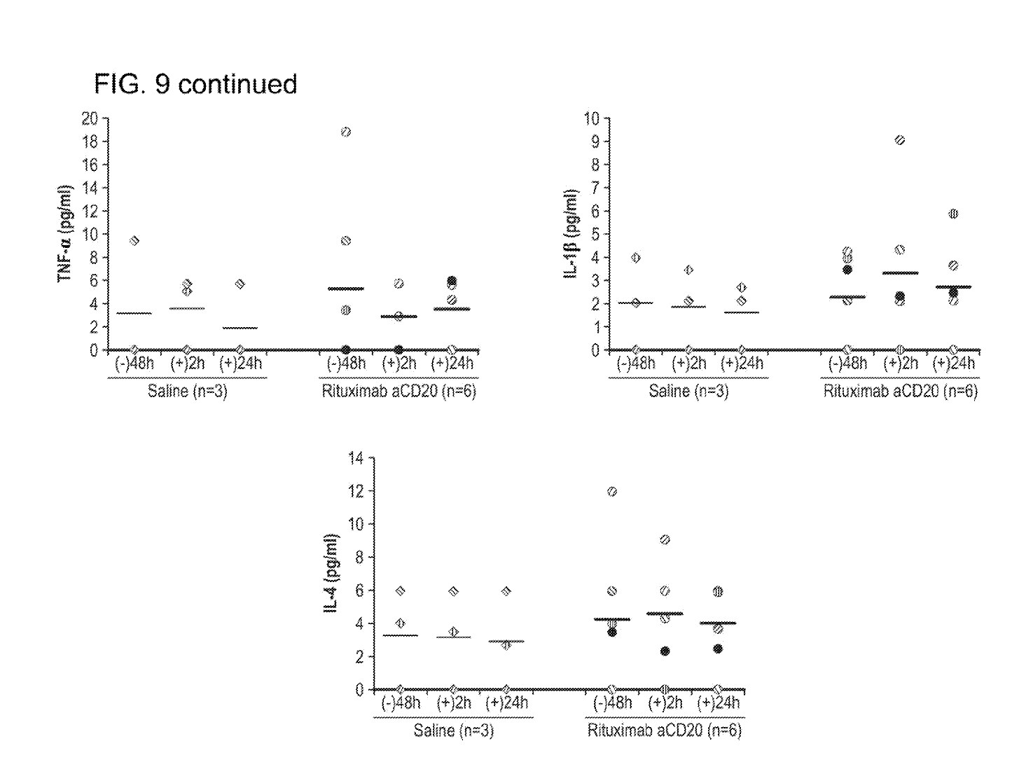

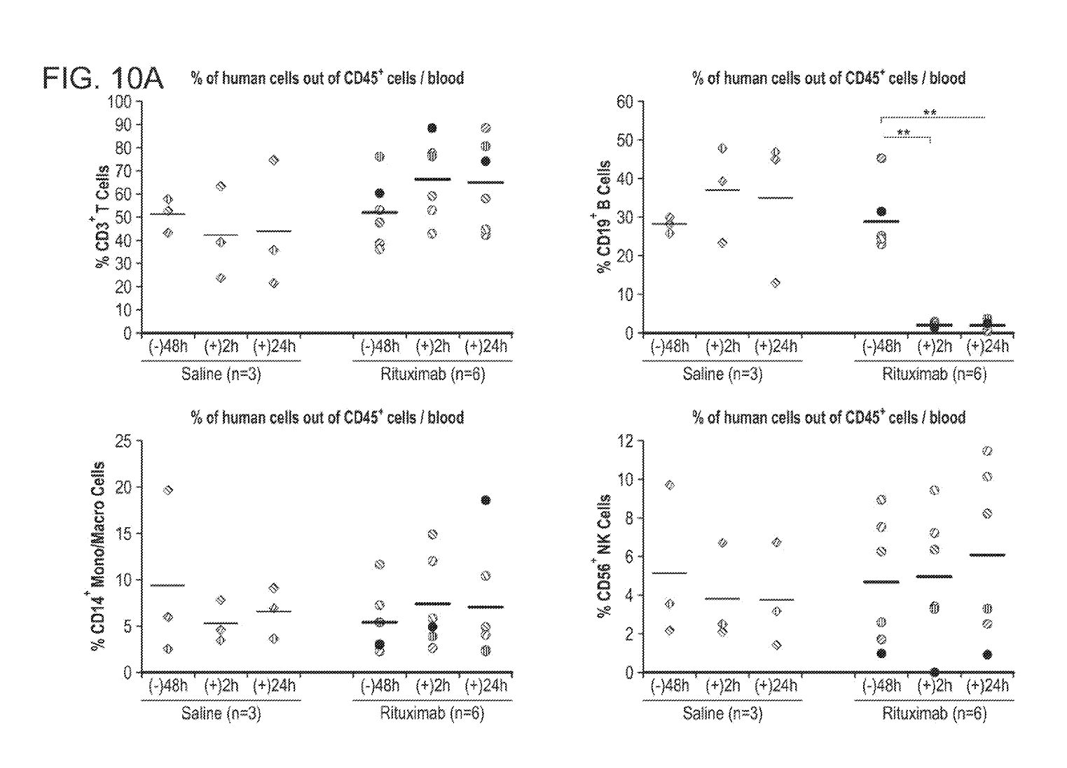

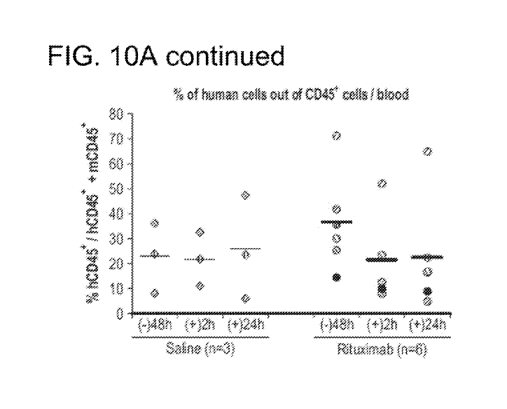

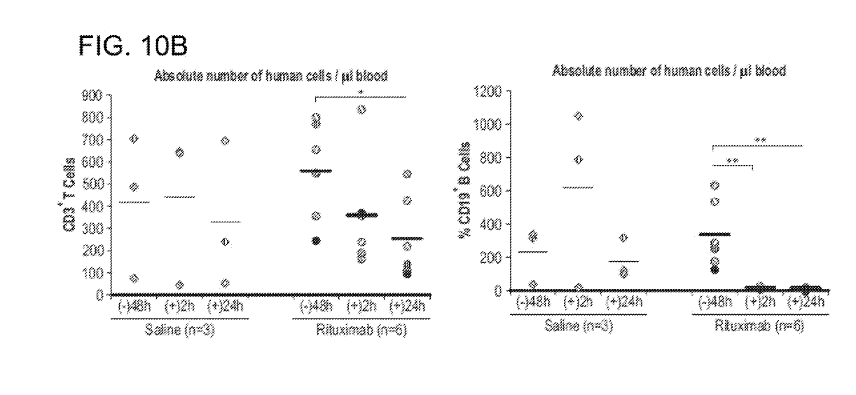

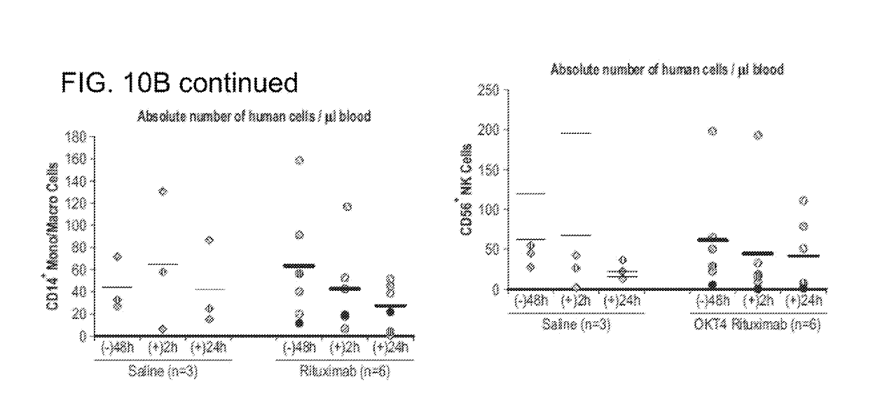

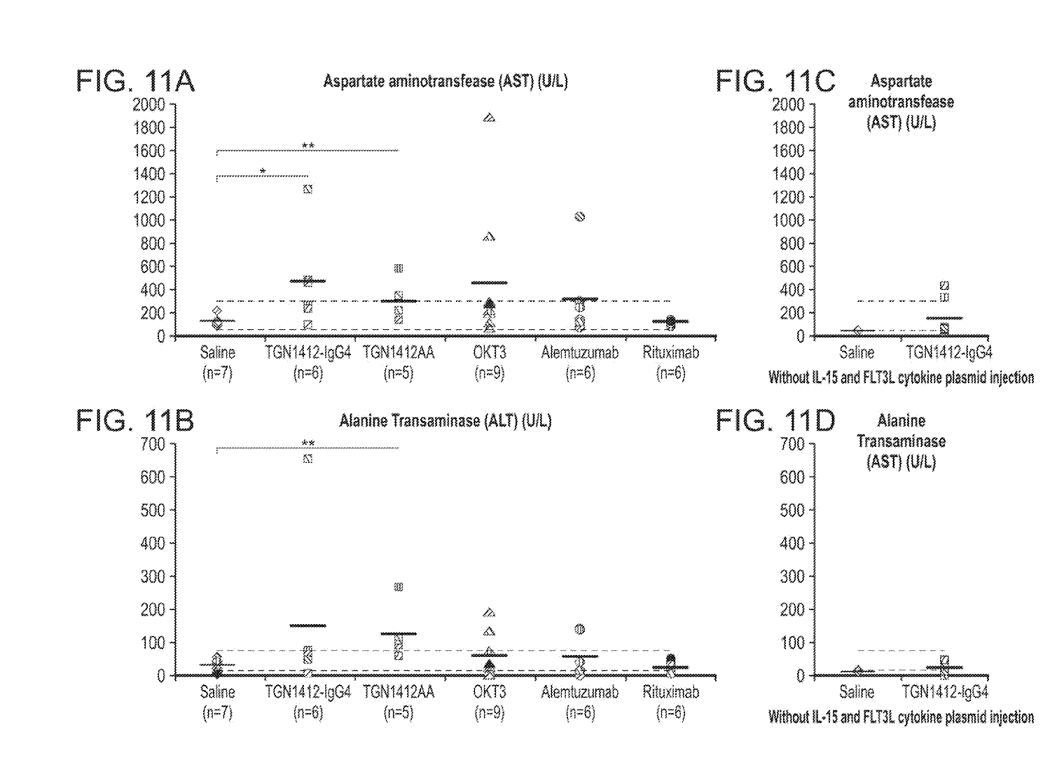

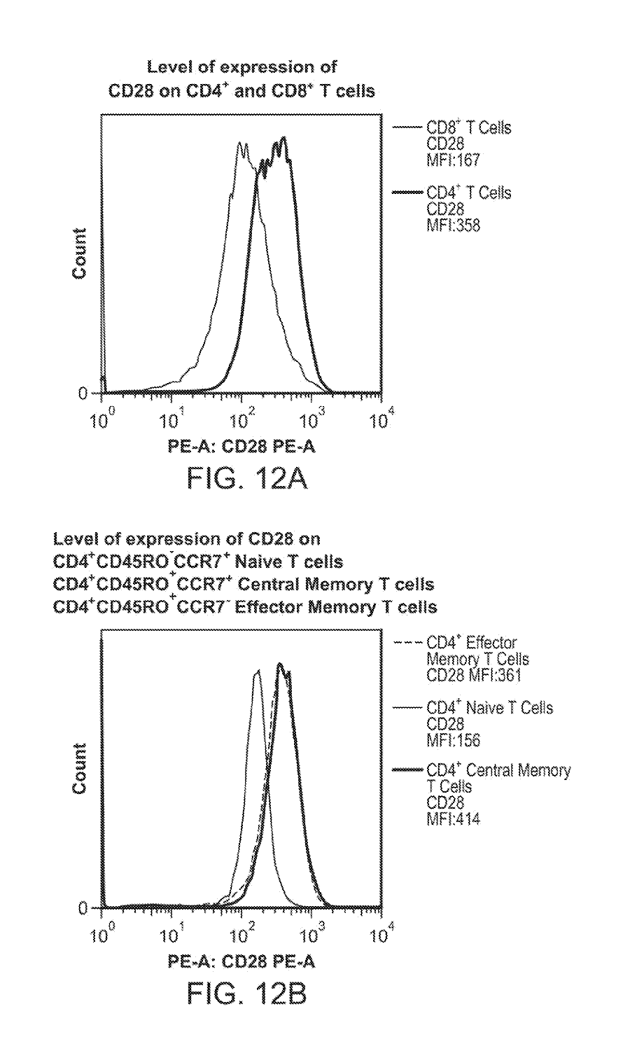

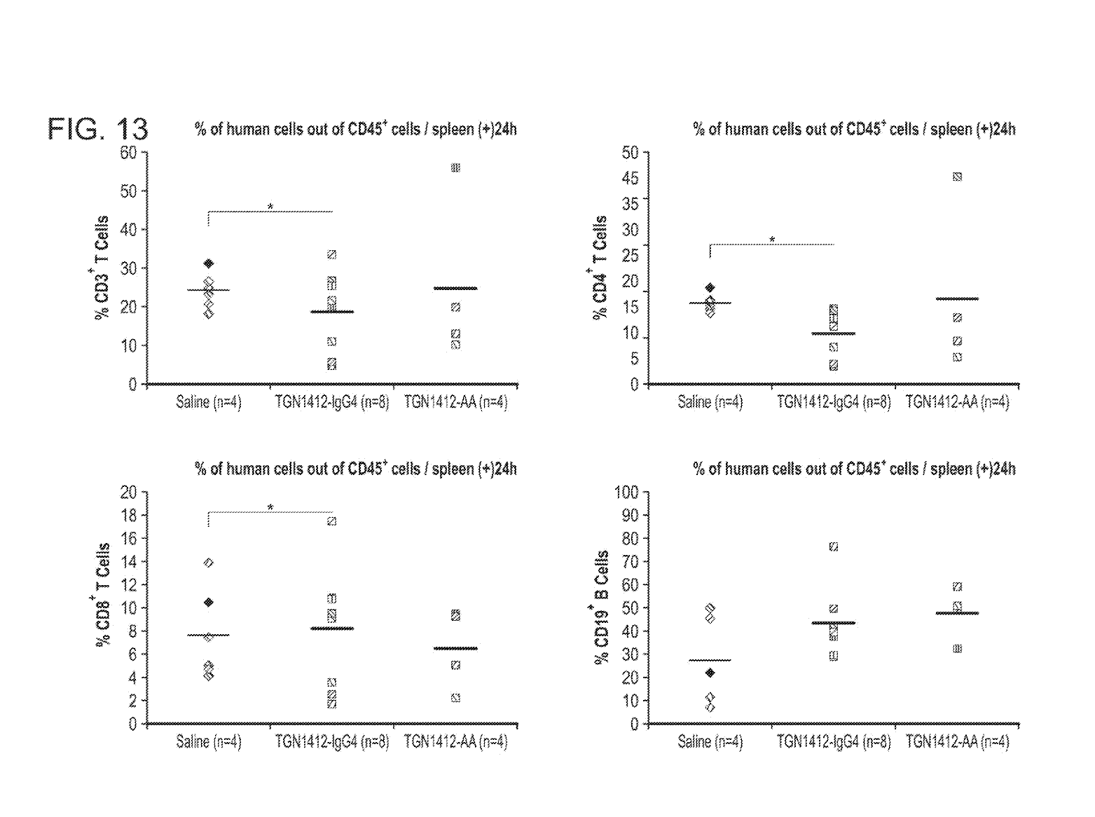

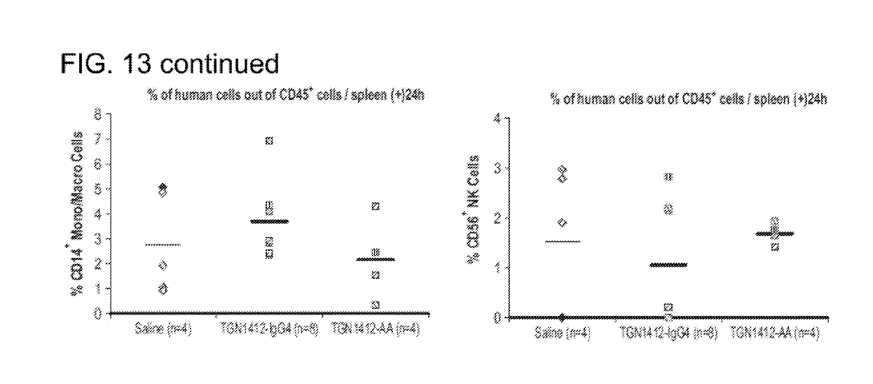

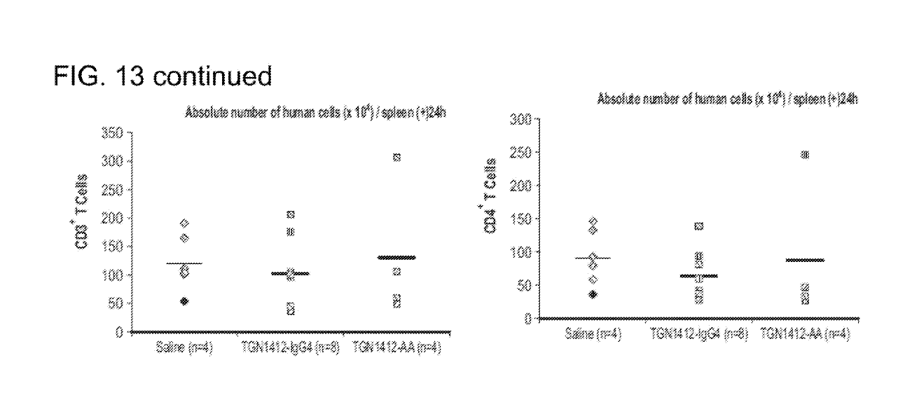

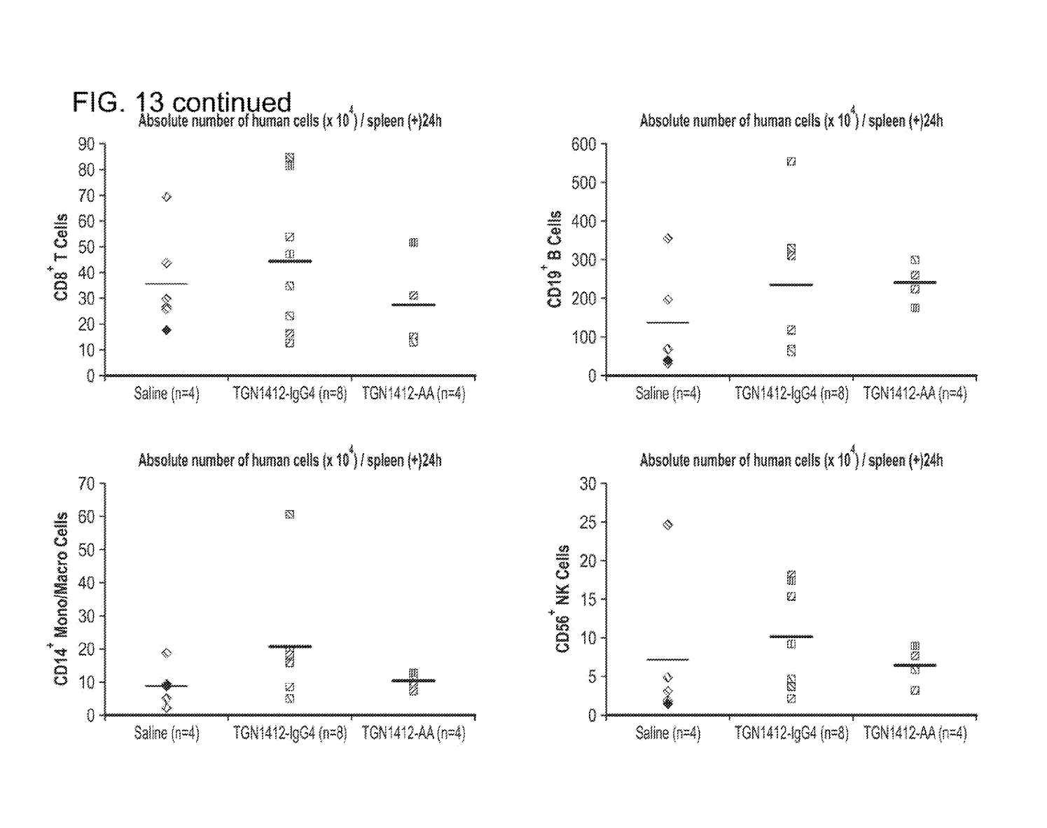

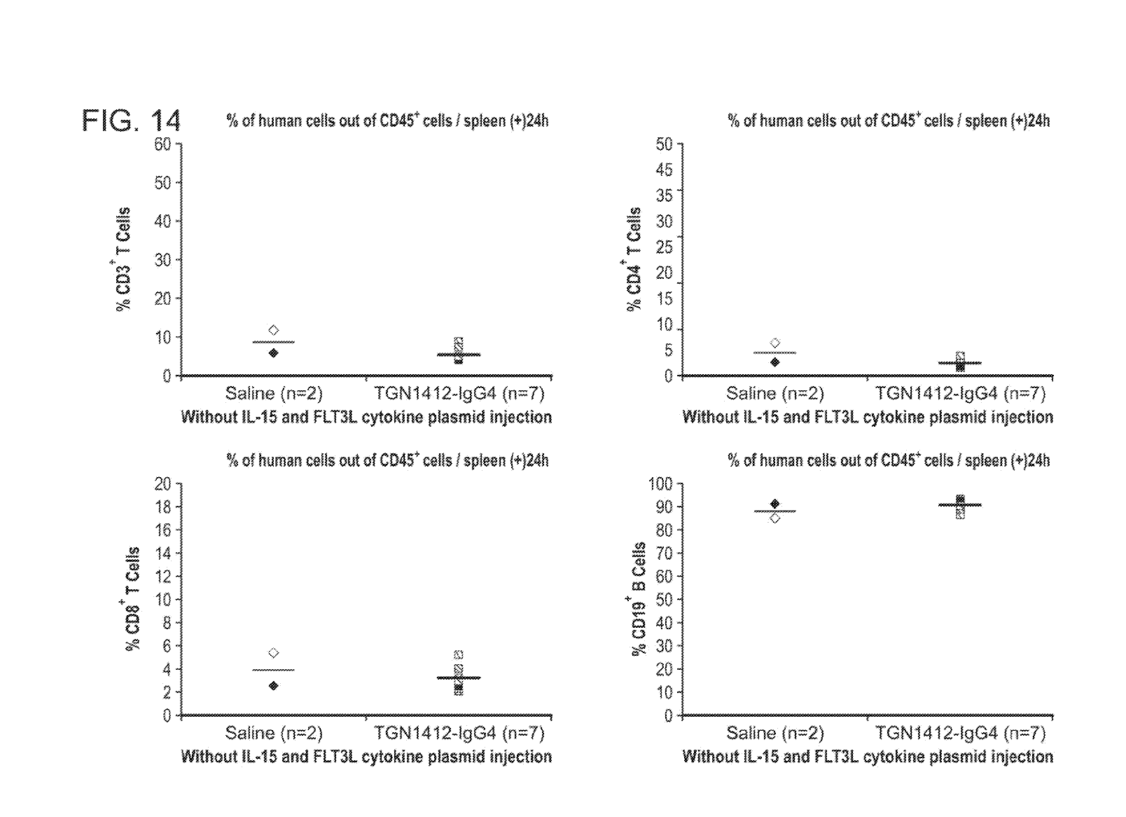

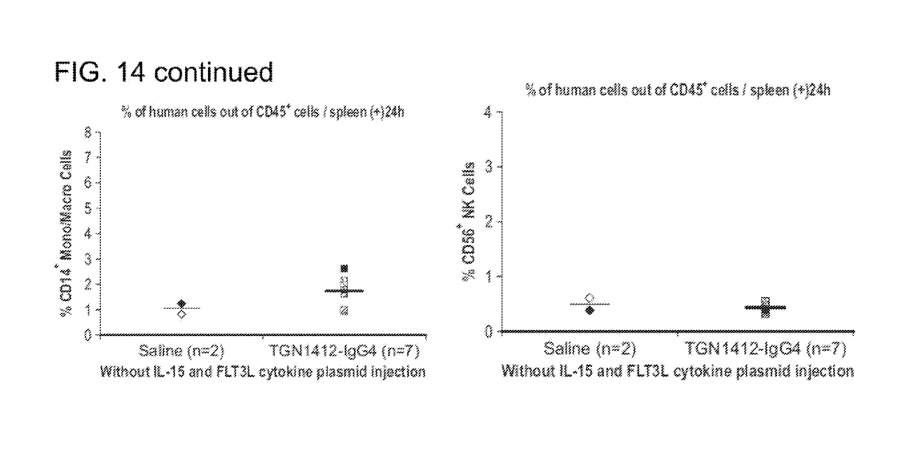

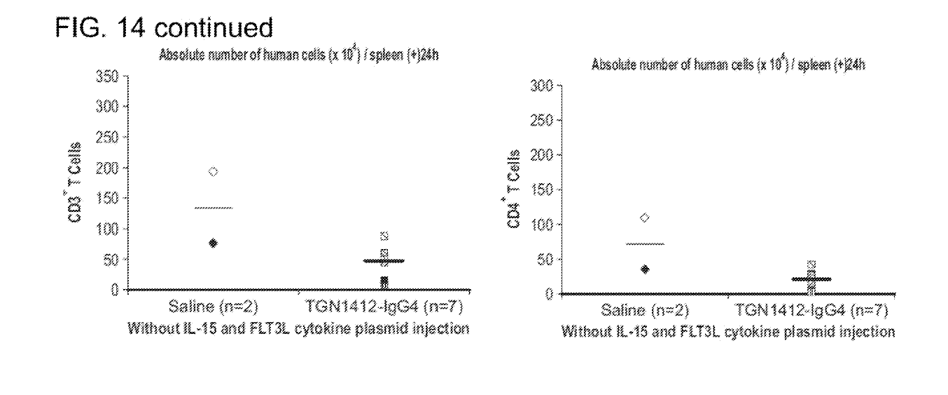

### DETAILED DESCRIPTION OF THE INVENTION

As described herein, cytokine-treated humanized (CTH) mice were generated and evaluated for the ability to predict immune toxicity of agent (e.g., therapeutics) that have been observed, for example, in the clinics. The cytokine-treated humanized mice were injected with different therapeutic antibodies, TGN1412 (anti-CD28), OKT3 (anti-CD3), alemtuzumab (anti-CDS2) and Rituximab (anti-CD20), and the adverse effects were assayed by cell immunophenotyping, cytokine and liver enzyme measurements. The therapeutic effects and side effects of the four antibodies in humanized mice were comparable to those observed in the clinic. Both TGN1412 and OKT3-treated mice showed a significant increase in major inflammatory cytokines accompanied by a depletion of T cells, a decrease in NK cell counts and an increase in liver enzymes. Alemtuzumab-treated mice exhibited a dramatic depletion of lymphocytes, natural killer cells and monocytes but only a slight increase (significant for IL-8) in cytokines levels. These responses were absent in the Rituximab-treated mice, where a depletion of B cells was noted. Administration of non-cytokine treated mice did not induce the cytokine responses. These findings show that CTH mice can predict immune toxicity of agents such as therapeutics with high fidelity and accuracy.

Accordingly, in one aspect, the invention is directed to a method of determining whether an (one or more) agent causes toxicity in a human. The method comprises administering the agent to a non-human mammal that has been engrafted with human hematopoietic stem cells (HSCs) and administered one or more human cytokines; and determining whether the agent causes toxicity in the non-human mammal, wherein if the agent causes toxicity in the non-human mammal then the agent causes toxicity in a human.

In another aspect, the invention is directed to a method of determining whether an (one or more) agent causes immune toxicity in a human. The method comprises administering the agent to a non-human mammal that has been engrafted with human hematopoietic stem cells (HSCs) and administered one or more human cytokines; and determining whether the agent causes immune toxicity in the non-human mammal, wherein if the agent causes immune toxicity in the non-human mammal then the agent causes toxicity in a human.

As used herein, an agent is toxic or causes toxicity if it causes injury or harm to an individual (e.g., human). The toxicity can be, for example, acute or chronic. In particular aspects, the invention is directed to methods of determining toxicity caused by human immune cells reconstituted in a humanized non-human mammal such as a humanized mice (e.g., cytokine-treated humanized mice). The advantage of using the humanized mice as described herein is that it allows the exploration or determination of in vivo toxicity caused by the reaction of a human immune system (that has been reconstituted in a non-human mammal such as a mouse) to an agent such as a therapeutic agent.

Immune toxicity refers to the undesirable/unintended effect of an agent on the functioning of the immune system of an individual. See, for example, Weir, A, Journal of Immunotoxicology, 5:3-10 (2008); Gribble, E J., et al., Expert Opinion Drug Metab Toxicol, 3(2) (2007).

In some instances, immune toxicity can produce a cytokine storm in an individual. Cytokine storm, cytokine release syndrome, or infusion reaction is an adverse event usually seen upon first exposure to an agent (e.g., a therapeutic mAb). It is characterized by the systemic release of several inflammatory cytokines. Symptoms range from mild to severe, including fatigue, headache, urticaria, pruritus, bronchospasm, dyspnea, sensation of tongue or throat swelling, rhinitis, nausea, vomiting, flushing, fever, chills, hypotension, tachycardia and asthenia. See, for example, Wing, M., et al. Journal of Immunotoxicology, 5:11-15 (2008) and Wang, H., et al., American Journal of Emergency Medicine, 26:711-715 (2008).

Thus, in yet another aspect, the invention is directed to a method of determining whether administration of an (one or more) agent will cause cytokine release syndrome in an individual (e.g., human) in need thereof. The method comprises administering the agent to a non-human mammal that has been engrafted with human hematopoietic stem cells (HSCs) and administered one or more human cytokines; and determining whether the agent causes cytokine release syndrome in the non-human mammal, wherein if the agent causes cytokine release syndrome in the non-human mammal then the agent will cause cytokine release syndrome in the human.

As used herein, HSCs (e.g., human HSCs) are self renewing stem cells that, when engrafted into a recipient, “repopulate” or “reconstitute” the hematopoietic system of the graft recipient (e.g., a non-human mammal; an immunodeficient non-human mammal) and sustain (e.g., long term) hematopoiesis in the recipient. Thus, when human HSCs are engrafted into a non-human mammal, the human HSCs repopulate the hematopoietic system of the non-human mammal with human HSCs.

HSCs are multipotent stem cells that give rise to (differentiate into) blood cell types including myeloid (e.g., monocytes and macrophages, neutrophils, basophils, eosinophils, erythrocytes, megakaryocytes/platelets, dendritic cells) and lymphoid lineages (e.g., T-cells, B-cells, NK-cells). The reconstituted human HSCs can differentiate into human NK cells, human monocytes, human macrophages, human dendritic cells, human red blood cells, human B cells, human T cells or combinations thereof in the non-human mammal.

HSCs express the cell marker CD34 and are commonly referred to as “CD34+”. As understood by those of skill in the art, HSCs can also express other cell markers, such as CD133 and/or CD90 (“CD133+”, “CD90+”). In some instances, HSCs are characterized by markers that are not expressed, e.g., CD38. Thus, in one embodiment of the invention, the human HSCs used in the methods described herein are CD34+, CD90+, CD133+, CD34+CD38−, CD34+CD90+, CD34+CD133+CD38−, CD133+CD38−, CD133+CD90+CD38−, CD34+CD133+CD90+CD38−, or any combination thereof. In a particular embodiment, the HSCs are both CD34 (“CD34+”) and CD133+ (“CD133+”), also referred to herein as “double positive” or “DP” cells or “DPC”. In another embodiment, the HSCs are CD34+CD133+, and can further comprise CD38− and/or CD90+.

HSCs are found in bone marrow such as in femurs, hip, ribs, sternum, and other bones of a donor (e.g., vertebrate animals such as mammals, including humans, primates, pigs, mice, etc.). Other sources of HSCs for clinical and scientific use include umbilical cord blood, placenta, fetal liver, mobilized peripheral blood, non-mobilized (or unmobilized) peripheral blood, fetal liver, fetal spleen, embryonic stem cells, and aorta-gonad-mesonephros (AGM), or a combination thereof.

As will be understood by persons of skill in the art, mobilized peripheral blood refers to peripheral blood that is enriched with HSCs (e.g., CD34+ cells). Administration of agents such as chemotherapeutics and/or G-CSF mobilizes stem cells from the bone marrow to the peripheral circulation. For example, administration of granulocyte colony-stimulating factor (G-CSF) for at least, or about, 5 days mobilizes CD34+ cells to the peripheral blood. A 30-fold enrichment of circulating CD34+ cells is observed with peak values occurring on day 5 after the start of G-CSF administration. Without mobilization of peripheral blood, the number of circulating CD34+ cells is very low, estimated between 0.01 to 0.05% of total mononuclear blood cells.

The human HSCs for use in the methods can be obtained from a single donor or multiple donors. In addition, the HSCs used in the methods described herein can be freshly isolated HSCs, cryopreserved HSCS, or a combination thereof.

As known in the art, HSCs can be obtained from these sources using a variety of methods known in the art. For example, HSCs can be obtained directly by removal from the bone marrow, e.g., in the hip, femur, etc., using a needle and syringe, or from blood following pre-treatment of the donor with cytokines, such as granulocyte colony-stimulating factor (G-CSF), that induce cells to be released from the bone marrow compartment.

The HSCs for use in the methods of the invention can be introduced into the non-human mammal directly as obtained (e.g., unexpanded) or manipulated (e.g., expanded) prior to introducing the HSCs into the non-human mammal. In one embodiment, the HSCs are expanded prior to introducing the HSCs into the non-human mammal. As will be appreciated by those of skill in the art there are a variety of methods that can be used to expand HSCs (see e.g., Zhang, Y., et al., Tissue Engineering, 12(8):2161-2170 (2006); Zhang C C, et al., Blood, 111(7):3415-3423 (2008)). In a particular embodiment, a population of HSCs can be expanded by co-culturing the HSCs with mesenchymal stem cells (MSCs) in the presence of growth factors (e.g., angiopoietin-like 5 (Angplt5) growth factor, IGF-binding protein 2 (IGFBP2), stem cell factor (SCF), fibroblast growth factor (FGF), thrombopoietin (TPO), or a combination thereof) to produce a cell culture. The cell culture is maintained under conditions in which an expanded population of HSCs is produced (e.g., see Khoury, M, Stem Cell Dev., 2(8):1371-1381 (2011) and International Application No. WO 2010/138873 which is incorporated herein by reference).

In the methods described herein, the agent is administered to a non-human mammal that has been engrafted with human HSCs and administered one or more human cytokines. The one or more human cytokines that are administered to the non-human mammal can be a (one or more) cytokine protein and/or a (one or more) nucleic acid (e.g., DNA, RNA) encoding one or more human cytokines. The human cytokines are administered or introduced into the non-human mammal to induce differentiation of the human HSCs into functional human cells (e.g., functional human blood cell lineages). As is known in the art, cytokines are proteins that stimulate or inhibit differentiation, proliferation or function of immune cells. Also known and available in the art are numerous human cytokine proteins and nucleic acid sequences which encode human cytokines (see, for example, www.ncbi.nlm.nih.gov). Methods for obtaining human cytokine protein and/or nucleic acid encoding one or more human cytokines are routine in the art and include isolating the protein or nucleic acid (e.g., cloning) from a variety of sources (e.g., serum), producing the protein or nucleic acid recombinantly, or obtaining the protein or nucleic acid from commercial sources.

There are a variety of human cytokines that can be used in the methods of the invention. Examples of such human cytokines include interleukin-12 (IL-12), interleukin-15 (IL-15), Fms-related tyrosine kinase 3 ligand (Flt3L), Flt3L/Flk2 ligand (FL), granulocyte macrophage colony stimulating factor (GM-CSF), interleukin-4 (IL-4), interleukin-3 (IL-3), macrophage colony stimulating factor (M-CSF), erythropoietin (EPO), interleukin-23 (IL-23), interleukin-3 (IL-3), interleukin-9 (IL-9), stem cell factor, interleukin-7 (IL-7), interleukin-17 (IL-17) and a combination thereof. The type of cytokine and the number of cytokines introduced into the non-human mammal will depend upon which human blood cell lineages are to be reconstituted when differentiation of the human HSCs occur in the non-human mammal.

In some aspects, at least (comprising) one cytokine, at least 2 cytokines, at least 3 cytokines, at least 4 cytokines, at least 5 cytokines, at least 6 cytokines, at least 7 cytokines, at least 8 cytokine, at least 9 cytokines, at least 10 cytokines, at least 11 cytokines, at least 12 cytokines, at least 13 cytokines, at least 14 cytokines, at least 15 cytokines, at least 16 cytokines, at least 17 cytokines, at least 18 cytokines, at least 19 cytokines, or at least 20 cytokines, are introduced into the non-human mammal. In other aspect, only (consisting, consisting essentially of) one cytokine, 2 cytokines, 3 cytokines, 4 cytokines, 5 cytokines, 6 cytokines, 7 cytokines, 8 cytokine, 9 cytokines, 10 cytokines, 11 cytokines, 12 cytokines, 13 cytokines, 14 cytokines, 15 cytokines, 16 cytokines, 17 cytokines, 18 cytokines, 19 cytokines, or 20 cytokines are introduced into the non-human mammal. Each cytokine protein and/or nucleic acid encoding each human cytokine can be introduced simultaneously or sequentially (e.g., in the instances in which more than one cytokine is to be expressed in the non-human mammal, each nucleic acid encoding each cytokine can be introduced in its own single plasmid or vector, or can be introduced in multiple plasmids or vectors; alternatively, all the nucleic acid encoding the cytokines to be introduced can be introduced in a single plasmid or vector).

In the methods of the invention, the human HSCs and human cytokine protein and/or nucleic acid encoding one or more human cytokines are introduced into a non-human mammal. As used herein, the terms “mammal” and “mammalian” refer to any vertebrate animal, including monotremes, marsupials and placental, that suckle their young and either give birth to living young (eutharian or placental mammals) or are egg-laying (metatharian or nonplacental mammals). Examples of mammalian species that can be used in the methods described herein include non-human primates (e.g., monkeys, chimpanzees), rodents (e.g., rats, mice, guinea pigs), canines, felines, and ruminents (e.g., cows, pigs, horses). In one embodiment, the non-human mammal is a mouse. The non-human mammal used in the methods described herein can be adult, newborn (e.g., <48 hours old; pups) or in utero.

In particular embodiments, the non-human mammal is an immunodeficient non-human mammal, that is, a non-human mammal that has one or more deficiencies in its immune system (e.g., NSG or NOD scid gamma (NOD.Cg-Prkdcscid Il2rgtm1Wjl/SzJ) mice) and, as a result, allow reconstitution of human blood cell lineages when human HSCs are introduced. For example, the non-human mammal lacks its own T cells, B cells, NK cells or a combination thereof. In particular embodiments, the non-human mammal is an immunodeficient mouse, such as a non-obese diabetic mouse that carries a severe combined immunodeficiency mutation (NOD/scid mouse); a non-obese diabetic mouse that carries a severe combined immunodeficiency mutation and lacks a gene for the cytokine-receptor γ chain (NOD/scid IL2R γ−/− mouse); and a Balb/c rag−/−γc−/− mouse.

Other specific examples of immunodeficient mice include, but are not limited to, severe combined immunodeficiency (scid) mice, non-obese diabetic (NOD)-scid mice, IL2rg^{—/—} mice (e.g., NOD/LySz-scid IL2rg^{-/-} mice, NOD/Shi-scid IL2rg^{—/—} mice (NOG mice), BALB/c-Rag^{—/—} IL2rg^{—/—} mice, H2^{d}-Rag^{—/—} IL2rg^{—/—} mice), NOD/Rag^{—/—} IL2rg^{—/—} mice.

In some embodiments, the non-human mammal is treated or manipulated prior to introduction of the human HSCs and human cytokines (e.g., protein and/or nucleic acid encoding one or more human cytokines to further enhance reconstitution of the human HSCs). For example, the non-human mammal can be manipulated to further enhance engraftment and/or reconstitution of the human HSCs. In one embodiment, the non-human mammal is irradiated prior to introduction of the HSCs and the human cytokines. In another embodiment, one or more chemotherapeutics are administered to the non-human mammal prior to introduction of the HSCs and the human cytokines.

As will also be appreciated by those of skill in the art, there are a variety of ways to introduce HSCs, human cytokine protein and nucleic acid encoding cytokines into a non-human mammal. Examples of such methods include, but are not limited to, intradermal, intramuscular, intraperitoneal, intraocular, intrafemoral, intraventricular, intracranial, intrathecal, intravenous, intracardial, intrahepatic, intra-bone marrow, subcutaneous, topical, oral and intranasal routes of administration. Other suitable methods of introduction can also include, in utero injection, hydrodynamic gene delivery, gene therapy, rechargeable or biodegradable devices, particle acceleration devises (“gene guns”) and slow release polymeric devices.

The HSCs can be introduced into the non-human using any such routes of administration or the like. In a particular embodiment, the HSCs are injected intracardially into the non-human mammal.

The one or more human cytokine proteins and/or nucleic acid encoding the one or more human cytokines can be also by introduced using any such route of administration. In the embodiment in which nucleic acid is introduced, any route of administration can be used as long as the nucleic acid(s) is/are expressed in the non-human mammal. For example, nucleic acid encoding the one or more cytokines can be introduced as naked nucleic acid (naked DNA), in a plasmid (e.g., pcDNA3.1(+)) or in a viral vector (e.g., adenovirus, adeno-associated virus, lentivirus, retrovirus and the like). In a particular embodiment, the nucleic acid encoding the one or more cytokines is introduced in a plasmid using hydrodynamic injection (e.g., into tail vein of a non-human mammal).

As will be appreciated by those of skill in the art, alternative methods can be used to introduce one or more human cytokines into the non-human mammal. For example, a knock-in methodology can be used. In molecular cloning and biology, a knock-in (or gene knock-in) refers to a genetic engineering method that involves the insertion of a protein coding cDNA sequence at a particular locus in an organism's chromosome. Typically, this is done in mice since the technology for this process is more refined, and because mouse embryonic stem cells are easily manipulated. Human cytokine knock-in mice are mice in which specific mouse cytokine locuses are replaced by human cytokines so the mice produce these specific human cytokines instead of mouse cytokines. See. for example, Willinger, T., et al., PNAS, 108(6):2390-2395 (2011) and Rongvaux, A., et al., PNAS, 108(6):2378-2383 (2011).

In addition, one or more human cytokines can be introduced into a non-human mammals using transgenic techniques. Transgenic mice have inserted DNA that originated from human or other species. The difference between knock-in technology and transgenic technology is that a knock-in involves a gene inserted into a specific locus, and is a “targeted” insertion. See, for example, Billerbeck, E., et al., Blood, 117(11) (2011).

The HSCs, human cytokine protein and/or the nucleic acid encoding the one or more human cytokines are introduced simultaneously or sequentially. In a particular aspect, the human HSCs are introduced into the non-human mammal and the non-human is maintained under conditions in which the human HSCs repopulate the hematopoietic system of the non-human mammal. After the hematopoietic system of the non-human mammal is reconstituted with human HSCs, human cytokines are then introduced into the non-human mammal. In a particular embodiment, human HSCs are introduced into a newborn pup (e.g., about 48 hours old) and human cytokine protein and/or nucleic acid encoding one or more human cytokines are introduced about 1 month, about 2 months, about 3 months, about 4 months, about 5 months, about 6 months, about 7 months, about 8 months, about 9 months, about 10 months, about 11 months, about 12 months later.

Once the HSCs and the one or more human cytokines are introduced, the non-human mammal is maintained under conditions in which the non-human is reconstituted with the human HSCs and the cytokines stimulate differentiation, proliferation and/or function of human immune cells in the non-human mammal. Such conditions under which the non-human animals of the invention are maintained include meeting the basic needs (e.g., food, water, light) of the mammal as known to those of skill in the art.

The methods described herein can further comprise determining whether the nucleic acid encoding the one or more human cytokine is expressed, the human HSCs are present and/or the human HSCs have differentiated into one or more human blood lineage cells. Methods for determining whether the nucleic acid is expressed and/or the non-human is reconstituted with the HSCs are provided herein and are well known to those of skill in the art. For example, flow cytometry analysis using antibodies specific for surface cell markers of human HSCs can be used to detect the presence of human HSCs in the non-human mammal. In addition, sera can be collected from the non-human mammal and assayed for the presence of the human cytokines. Assays for assessing the function of the differentiated HSCs (e.g., NK cells, dendritic cells, T cell, B cells, monocytes/macrophages, erythrocytes) can be also be used. See, for example, International Published Application No. WO 2011/002727 which is incorporated herein by reference in its entirety.

As will be appreciated by one of skill in the art, in addition to cytokines, other proteins and/or nucleic acid encoding other proteins (e.g., human proteins; human secreted proteins), such as growth factors, steroids, and/or small molecules, can be used in the methods to improve reconstitution and/or function of human cells beyond blood lineage cells. For example, an agonist of one or more of the human cytokines can be introduced into the non-human mammal to enhance reconstitution of the HSCs.

The methods described herein can be used to assess a variety of agents for toxicity in humans. For example, the agent can be a small molecular weight organic or inorganic molecule, therapeutic agent, diagnostic agent, cosmetic agent, and/or alimentary additive agent. Specific examples of agents include an antibody (e.g., polyclonal antibody, monoclonal antibody, chimeric antibody, humanized antibody and the like), protein, nucleic acid, polysachharide, a lipopolysaccharide, a lipoprotein, a lipid, a vaccination agent (e.g., a microbial antigen), a nanoparticle etc.

The agent can be administered to the non-human mammal using any of a variety of routes of administration. Examples of routes of administration include intradermal, intramuscular, intraperitoneal, intraocular, intrafemoral, intraventricular, intracranial, intrathecal, intravenous, intracardial, intrahepatic, intra-bone marrow, subcutaneous, topical, oral and intranasal routes of administration. Other suitable methods of introduction can also include, hydrodynamic gene delivery, gene therapy, rechargeable or biodegradable devices, particle acceleration devises (“gene guns”) and slow release polymeric devices.

As will be appreciated by those of skill in the art, a variety of methods can be used to determine whether the agent causes toxicity, immune toxicity, and/or cytokine storm in the non-human mammal. For example, whether the agent causes toxicity in the non-human mammal is determined by measuring cell surface markers, immune cell phenotype (e.g., an immune cell phenotype that is indicative of toxicity (immune toxicity) in, for example, a human), increased expression of one or more liver enzymes, increased expression of one or more pro-inflammatory cytokines or a combination thereof that occurs in the non-human mammal after administration of the agent.

As will be appreciated by those of skill in the art, measuring immune cell phenotype can be determined in a variety of ways. For example, immune cell phenotype can be measured by determining proliferation (increased; decreased) and/or activation (increased; decreased) of one or more immune cells produced in the non-human mammal. The immune cells can be human immune cells, mouse immune cells or a combination thereof. In a particular embodiment, immune cell phenotype is measured by determining proliferation (increased; decreased) and/or activation (increased; decreased) of one or more human immune cells produced in the non-human mammal. Examples of immune cells (human; mouse) include lymphocytes (e.g., T cell, B cells), natural killer (NK) cells, monocytes, macrophages, CD45.1⁺ cells and the like.

Proliferation of T cells can be determined by measuring cells expressing CD3⁺, proliferation of B cells can be determined by measuring cells expressing CD19⁺, proliferation of NK cells can be determined by measuring cells expressing CD56⁺, and proliferation of monocytes/macrophages can be determined by measuring cells expressing CD14⁺. In addition, proliferation of T cells can be determined by measuring cells expressing CD45+CD3+, proliferation of B cells can be determined by measuring cells expressing CD45+CD19+, proliferation of NK cell can be determined by measuring cells expressing CD45+CD56+, proliferation of lymphocytes cane be determined by measuring cells expressing CD45+CD56+, and proliferation of monocytes/macrophages can be determined by measuring cells expressing CD45+CD14+.

Antibodies that specifically bind these markers of immune cells can be used for detection. In addition, or alternatively, immunochemistry can be used to detect infiltration of one or more organs in the non-human mammal by human cells expressing these markers. Immunomagnetic cell separation can also be used to quantify the different immune cell types.

As will also be understood by those in the art, activation of immune cells (e.g., human or mouse) can also be used to determine whether the agent causes toxicity. For example, activated T cells are indicated by increased expression of CD69, CD25, CD44 and decrease expression of CD62L antigens.

Methods for detecting or measuring increased expression of one or more liver enzymes (mouse or human) in the non-human mammal are also known in the art. For example, known methods include those that detect aspartate and/or alanine aminotransferase.

Similarly, methods for measuring increased expression of one or more pro-inflammatory cytokines (human or mouse) are also known to those skilled in the art. Pro-inflammatory human cytokines include interleukin-2 (IL)-2, IL-6, IL-8, IL-1β, IL-4, gamma interferon (IFN-γ), tumor necrosis factor alpha (TNF-α) IL-10 or a combination thereof.

Increased expression of pro-inflammatory cytokines can be determined as described herein using flow cytometry. Specifically, pro-inflammatory cytokines were detected in sera using a BD Cytometric Bead Array (CBA) (BD Biosciences, USA). The experiments were conducted according to the manufacturer's recommendation and results were analyzed with the FCAP array software (Soft Flow Hungary, BD Biosciences). Alternatively, antibody-based methods and/or enzyme-linked immunosorbent assays can be used.

Other ways to measure toxicity (e.g., immune toxicity) in the non-human mammal include obtaining body weight measurements, and/or analyzing histology sections (liver, kidney, and lung), blood parameters (creatinine, high-sensitivity CRP (CRPHS), albumin and blood urea nitrogen (BUN); measure platelets (decrease); and increase D-D Dimer (increase).

Whether the agent causes toxicity in the non-human mammal can be determined at one or more time points after administration of the agent to the non-human mammal. For example, whether the agent causes toxicity in the non-human mammal can be determined within one or more hours (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours 24 hours), one or more days (e.g., 2 days, 3 days, 4 days, 5 days, 6 days, 7 days, 8 days, 9, days, 10 days, 11 days, 12 days, 13 days, 14 days, 15 days, 16 days, 17 days, 18 days, 18 days, 19 days, 20 days, 21 days, 22 days, 23 days, 24 days, 25 days, 27 days, 28 days, 29 days, 30 days, 31 days, 32 days, 33 days, 34 days, 35 days, 36 days, 37 days, days, 34 days, 35 days, 36 days, 37 days, 38 days, 39 days, 40 days), one or more weeks (e.g., 1 week, 2 weeks, 3 weeks, 4 weeks, 5 weeks, 6 weeks, 7 weeks, 8 weeks, 9 weeks, 10 weeks, 11 weeks, 12 weeks), one or more months (e.g., 1 month, 2 months, 3 moths, 4 months, 5 months, 6 months), or one or more years (e.g., 1 year, 2 years) etc. after administration of the agent.

The methods of the invention can further comprise comparing the effects in the non-human mammal that has been administered the agent to a suitable control. For example, a suitable control would be a non-human mammal that has been engrafted with human HSCs and treated with (administered; introduced) human cytokines, but not administered the agent.

To date there have been a few in vitro assay using whole blood or PBMC from humans to screen for cytokine release syndrome. Monoclonal antibodies were tested in aqueous solution or immobilized directly onto plastic plate by directly air drying or wet coating, and indirectly via anti-Fc capture. The most successful method of application, in term of stimulating the release of large cytokines was air drying of the mAbs onto plates or adding soluble mAbs to cultures with endothelial cell monolayers. The use of humanized mice more closely mimics the human environment allowing not only the measurement of the cytokine release but also other immunologic parameters, such as immunophenotyping, proliferation, activation of the different sub type of immune cells and the biochemistry parameter, and therefore, should provide results that more closely reassemble what happen when mAbs are given to patients and thus have superior predictive value.

Current clinical testing of new drugs on volunteers results in 90% of drugs failing. This failure is often due to toxicities that were not exposed in preclinical studies largely due to the inadequacy of the existing animal and primate models because of differences in the immune system between human and animal/primate. The commercial value of an animal model that can accurately predict these adverse effects arc immense in regards with the investment risk assessment. Moreover, many therapeutic candidates that successfully have gone through all in vitro and pre-clinical testing do not reach clinical testing phases as they were unable to secure appropriate funding. In this regard, the humanized model described herein can be used as a platform to prioritize with high degree of accuracy, the large number of clinically relevant candidates for clinical evaluation. Provided herein is strong evidence showing that the cytokine-treated humanized mice present a robust prediction tools for drug immunotoxicity testing. The methods described herein contribute to the estimate and set up of the first-dose-in-man, based on the ‘no observed adverse effect level’ (NOAEL) and on the ‘minimal anticipated biological effect level’ (MABEL) as determined in toxicity studies. Another application is to identify the different mechanisms that underlie the side effects and determine the most sensitive, and predictive common biological markers to use for the cytokine release syndrome in humans. As shown herein, humanized mice were not only able to show the expected side-effects of specific monoclonal antibodies but also confirmed their corresponding desired effect (for example, depletion of T or B cells). These advantages point to applications beyond drug safety, but also toward standard drug testing for molecules targeting the immune system. The model provided herein is the missing link between preclinical and clinical testing. The integration of this model into drug development paradigms has the potential to facilitate entry into first in human clinical trials and accelerate the process by which new therapeutics reach patients.

Although the invention of a robust prediction tool for drug immunotoxicity testing has been exemplified using humanized mice treated with IL-15 and Flt-3L, those of skill in the art will appreciate that other cytokines can be used, such as GMCSF and IL-4 can be used to treat the mice in order to increase the response of T cells, or MCSF in order to increase the number of monocytes as the cytokine release syndrome occurs also by the Fc end of the mAb to the Fc receptors on non-target cell to cause cytokine release or binding to the Fc receptor causes clustering and signaling through the target cell.

### EXEMPLIFICATION

### Example 1

### Methods

### Fetal Liver CD34⁺ (HSC) Cell Isolation

Human fetal livers were obtained from aborted fetuses at 15-23 weeks of gestation in accordance with the institute ethical guidelines. All women have given written informed consent for the donation of fetal tissue for research. The fetuses were collected under sterile condition within 2 h of the termination of pregnancy. The liver tissue from the fetus was initially cut into small pieces, followed by digestion with 2 mg/ml collagenase IV prepared in DMEM for 15 min at 37° C. with periodic mixing. Then, a single cell suspension was prepared by passing the digested tissue through 100 μm cell strainer (BD Biosciences). Viable cells were counted by excluding dead cells with Trypan blue. Cell isolation procedures were carried out under sterile condition using the CD34 positive selection kit (Stem Cell Technologies, Canada). The purity of CD34+ cells was determined by flow cytometry and rated between 80 to 95%.

### Construction of Humanized Mice and Hydrodynamic Gene Delivery

NSG mice were purchased from the Jackson Laboratories and maintained under specific pathogen-free conditions in the animal facilities at Nanyang Technological University and National University of Singapore. To reconstitute mice, newborn pups (less than 48 hours old) were irradiated with 100 cGy using a Gamma radiation source and injected intracardially with CD34+CD133+ cells (1×10 cells/recipient). Human IL-15 and human Fltr-3 ligand were cloned separately into pcDNA3.1(+) vector (Invitrogen, USA) as described previously (Chen Q et al. (2009) Proc Natl Acad Sci, USA, 106:21783-21788). For hydrodynamic gene delivery, 12 week-old humice were injected with 50 μg of each plasmid in a total of 2 ml saline within 7 seconds using a 27 gauge needle. All research with human samples and mice was performed in compliance with the institutional guidelines.

### Injection of Human Monoclonal Antibodies

The blood sampling at seven days post-cytokines treatment was used as the time point (−48 hours) for setting the baseline of the production levels of different cytokines and human cells absolute numbers and percentages. Two days later (0 hours), mice were then intra-venously injected (25 μg/mouse) with one of the corresponding monoclonal antibodies (mAbs), TGN1412-IgG4 also referred to herein as TGN1412-AA (anti-CD28, custom produced by JN Bioscences LLC, USA), OKT3 (anti-CD3, Biolegend), Campath® (Alemtuzumab, anti-CD52, Genzyme Corporation, Cambridge, Mass.) or Mabthera® (Rituximab, anti-CD20, Hoffmann-La Roche) resuspended in 200 μl of clinical grade (0.9%) sodium chloride solution (Braun). Blood samples were collected 2 and 24 hours after the mAb treatment, and all injected animals were euthanized at 24 hours.

### Single Cell Preparation, Cytokine Detection, Antibodies, and Flow Cytometry.

Single cell suspensions were prepared from spleen by standard procedures. The following human conjugated antibodies were used for flow cytometry staining: CD3, CD4, CD8, CD19, CD28, CD45, CD45RO, CCR7 from Biolegend; CD14 and CD56 from BD Biosciences (BD Biosciences, USA) and mouse CD45.1 from eBioscience. Cells were stained with appropriate antibodies in 100 μl PBS containing 0.2% BSA and 0.05% sodium azide for 30 min on ice. Flow cytometry was performed on a LSRII flow cytometer using the FACSDiva software (BD, Franklin Lakes, N.J.), and samples were analyzed using the Flowjo software. For cytokine detection, the concentration of human IL-2, IL-4, IL-6, IL-8, IL-1β, TNF-α, and IFN-γ were determined in sera using a BD Cytometric Bead Array (CBA) (BD Biosciences, USA). The experiments were conducted according to the manufacturer's recommendation and results were analyzed with the FCAP array software (Soft Flow Hungary, BD Biosciences).

### Statistics

Results were analyzed using GraphPad Prism 5.0 (Graph-Pad Softwares Inc., CA, USA).

### Results

As described herein, whether immunocompromised mice (e.g., NSG mice) engrafted with human HSCs and treated with human cytokines enhance the immune system and are able to serve as an accurate and reliable system for assaying and predicting toxicity in humans was investigated. Four monoclonal antibodies specific for CD28, CD3, CD52 and CD20 were used to validate the system because these antibodies are known to exhibit different side effects in humans. A major side effect is the “cytokine storm”. The cytokine release in treated mice was measured and compared to the secretion profiles of clinical data. Following TGN1412 injection, mice showed a significant increase in serum levels of human IL-2, IFN-γ, TNF-α and IL-8. This was not observed in humanized mice that were not treated with human cytokines. The cytokine treatment was used to obtain the cytokine responses described above. The kinetics of cytokine production was in good agreement with the expression curve depicted in the clinical trial, where all 6 volunteers manifested a similar trend. However, only 2 of the 6 recruits showed an increased level of 1L-4 and 3/6 for IL-1β. Similarly, only 2 of 9 mice showed an increased IL-4 level but no change in the IL-1β level. Besides the similarities observed at the cytokine level, T cell numbers dramatically decreased in both humans and humanized mice, and CD4⁺ cells seemed to be more profoundly affected probably because they showed a higher expression of the CD28. The single difference noted was the drop of the monocytes counts only in the clinical trial; this could be related to an initial difference in the monocytes numbers between the humanized mice and humans. It was noteworthy to mention that none of these symptoms is detected in the initial study in wild type mice prior to the clinical trial. The CD4 effector memory cells CD4⁺CD45RO⁺CCR7CD28⁺ were identified in the cytokine-treated humanized mice and found to be responsible for the production of pro-inflammatory cytokines following TGN1412 stimulation. This subset is specific to humans and absent in non-humans primates, and wild type mice, and can account for the absence of significant side effects detected in the conventional models.

Similar to the results obtained with TGNI412, the administration of OKT3 in the cytokine-treated humanized mice resulted in elevation of human IL-2, IL-6, IL-8 and IFN-γ cytokines. These findings were in parallel with results reported in kidney transplanted patients, treated with OKT3. The T cell depletion noted was also in accordance with the results in patients where lymphopenia and neutropenia were evident at two hours after the injection.

Alemtuzumab, known to induce a milder cytokine storm in patients, was also tested. The administration of Alemtuzumab to the cytokine-treated humanized mice induced an elevated level of human IL-2, IL-6, IL-8 and 1L-1β at 2 hrs but returned to the baseline level at 24 h, except for IL-1β. A severe reduction in B and T cells, NK cells and monocytes was observed 2 hrs after the injection. All these results were similar with what was observed in patients treated with Alemtuzumab.

Rituximab, a monoclonal antibody known for having only minimal or no inflammatory cytokine release in patients, in contrast with the severe adverse effects described for the TGN1214 and OKT3, was also tested. In one clinical study, it was shown that following Rituximab administration, some leukemic patients showed a slight rise of the IL-6 (˜80 pg/ml) and TNF-β (˜870 pg/ml) levels with minimal change in IL-2, IL-4 and IFN-γ compared to the baseline levels. The cytokine release was transient as both cytokine levels returned to baseline after completion of the initial Rituximab infusion. This induced expression was related to leukemic patients with large numbers of B lymphocytes. The absence of cytokines release in the study described herein could be related to the low number of CD20⁺ lymphocytes in comparison with the chronic lymphocytic leukemia patients. However, the model described herein is more comparable to the outcome noted in the rheumatoid arthritis patients, where a depletion of peripheral-blood B cells was noted. Although a reduction of the T cells number was observed 2 and 24 hours after mAb injection in accordance with the result of a clinical trial describing a transient decrease of the peripheral T cell counts post Rituximab infusion.

Together, these results show that the cytokine-treated humanized mice are predictive of acute immune toxicity of biologic therapeutics, including monoclonal antibodies and other protein therapeutics.

### Example 2

Provided below is additional data and a reanalysis of the experiments described in Example 1 in which whether the cytokine-treated humanized mice can accurately predict immune toxicity of antibody therapeutics was evaluated. As described in Example 1, four monoclonal antibodies with different degrees of side effect in humans were selected for analysis.

Besides TGN1412, OKT3, a mouse mAb against human CD3 for suppressing renal allograft rejection, is known to induce severe adverse effects, including cytokine release syndrome and an acute or severe influenza-like syndrome. Alemtuzumab, a humanized anti-CD52 antibody for treating chronic lymphocytic leukemia and preventing graft-versus-host disease, is also known to induce release of inflammatory cytokines. CD52 is a glycoprotein expressed on the surface of essentially all normal and malignant T and B lymphocytes, the majority of monocytes, macrophages and natural killer cells. Inflammatory cytokines release was also observed after first dose of Alemtuzumab. In patients with relapsed or refractory B-cell chronic lymphocytic leukemia, those with massive lymphadenopathy are more prone to cytokine release syndrome. Rituximab is a murine-human chimeric antibody that binds CD20 primarily located on pre-B and mature B lymphocytes. Rituximab result in an effective modulation of autoimmune diseases, and is also used for the treatment of leukemia and lymphomas, showing mild to no side-effects, largely depending on the nature and the importance of the tumor. The study described herein shows that results from the humanized mice accurately predicted the immune toxicity of four antibody therapeutics in humans.

### Material and Methods

### Fetal Liver CD34+ Cell Isolation

Human fetal livers were obtained from aborted fetuses at 15-23 weeks of gestation in accordance with the institute ethical guidelines (Polkinhorne). All women have given written informed consent for the donation of fetal tissue for research. The fetuses were collected under sterile condition within 2 h of the termination of pregnancy. The liver tissue from the fetus was initially cut into small pieces, followed by digestion with 2 mg/ml collagenase IV prepared in DMEM for 15 min at 37° C. with periodic mixing. Then, single cell suspension was prepared by passing the digested tissue through 100 μm cell strainer (BD Biosciences). Viable cells were counted by excluding dead cells with Trypan blue. Cell isolation procedures were carried out under sterile condition using the CD34 positive selection kit (Stem Cell Technologies, Canada). The purity of CD34+ cells was determined by flow cytometry and rated between 90 to 98%.

### Construction of Humanized Mice and Hydrodynamic Gene Delivery

NSG mice were purchased from the Jackson Laboratories and maintained under specific pathogen-free conditions in the animal facilities at Nanyang Technological University and National University of Singapore. To reconstitute mice, newborn pups (less than 48 hours old) were irradiated with 100 cGy using a Gamma radiation source and injected intracardially with CD34+CD133+ cells (1×10⁵ cells/recipient). Human IL-15 and human Fltr-3 ligand were cloned separately into pcDNA3.1(+) vector (Invitrogen, USA) as described previously. For hydrodynamic gene delivery, 12 week-old humice were injected with 50 μg of each plasmid in a total of 2 ml saline within 7 seconds using a 27 gauge needle. All research with human samples and mice was performed in compliance with the institutional guidelines.

### Injection of Human Monoclonal Antibodies

The blood sampling at seven days post-cytokines treatment was used as the time point (−48 hours) for setting the baseline of the production levels of different cytokines and human cells absolute numbers and percentages. Two days later (0 hours), mice were then intra-venously injected (25 μg/mouse) with one of the corresponding monoclonal antibodies (mAbs), TGN1412-IgG4 or TGN1412-AA (anti-CD28, custom produced by JN Bioscences LLC, USA), OKT3 (anti-CD3, Biolegend), Campath® (Alemtuzumab, anti-CD52, Genzyme Corporation, Cambridge, Mass.) or Mabthera® (Rituximab, anti-CD20, Hoffmann-La Roche) resuspended in 200 μl of clinical grade (0.9%) sodium chloride solution (Braun). Blood samples were collected 2 and 24 hours after the mAb treatment, and all injected animals were euthanized at 24 hours.

### Single Cell Preparation, Cytokine Detection, Antibodies, and Flow Cytometry.

Single cell suspensions were prepared from spleen by standard procedures. The following human conjugated antibodies were used for flow cytometry staining. CD3, CD4, CD8, CD19, CD28, CD45, CD45RO, CCR7 from Biolegend; CD14 and CD56 from BD Biosciences (BD Biosciences, USA) and mouse CD45.1 from eBioscience. Cells were stained with appropriate antibodies in 100 μl PBS containing 0.2% BSA and 0.05% sodium azide for 30 min on ice. Flow cytometry was performed on a LSRII flow cytometer using the FACSDiva software (BD, Franklin Lakes, N.J.), and samples were analyzed using the Flowjo software. For cytokine detection, the concentration of human IL-2, IL-6, IL-8, IL-1β, TNF-α, IFN-γ and mouse IL-2, IL-6, IL-8 were determined in sera using a BD Cytometric Bead Array (CBA) (BD Biosciences, USA). The experiments were conducted according to the manufacturer's recommendation and results were analyzed with the FCAP array software (Soft Flow Hungary, BD Biosciences).

### Statistics

Results were analyzed using GraphPad Prism 5.0 (Graph-Pad Softwares Inc., CA, USA).

### Results

### TGN1412 Induces Similar Adverse Side Effects in Cytokine-Treated Humanized Mice as in Humans

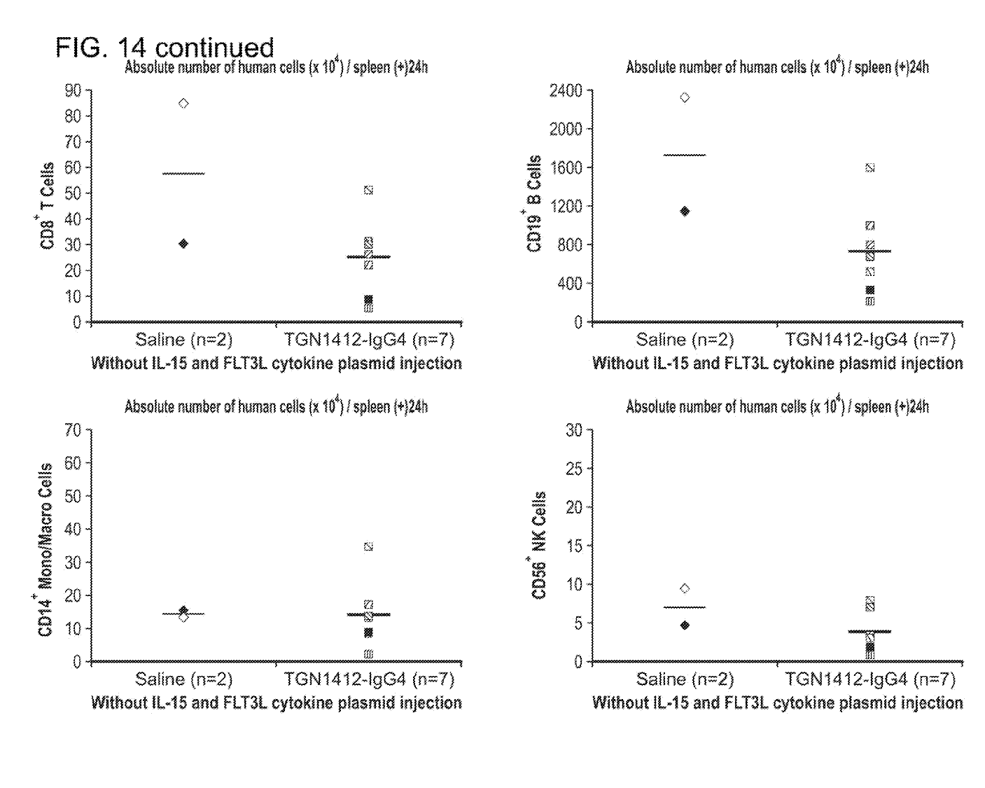

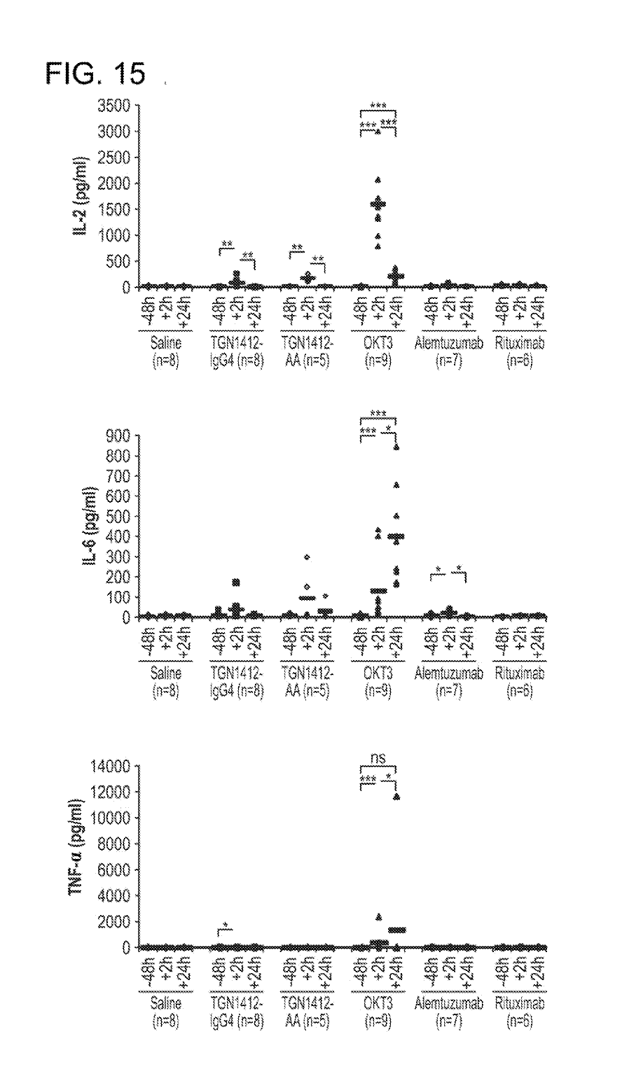

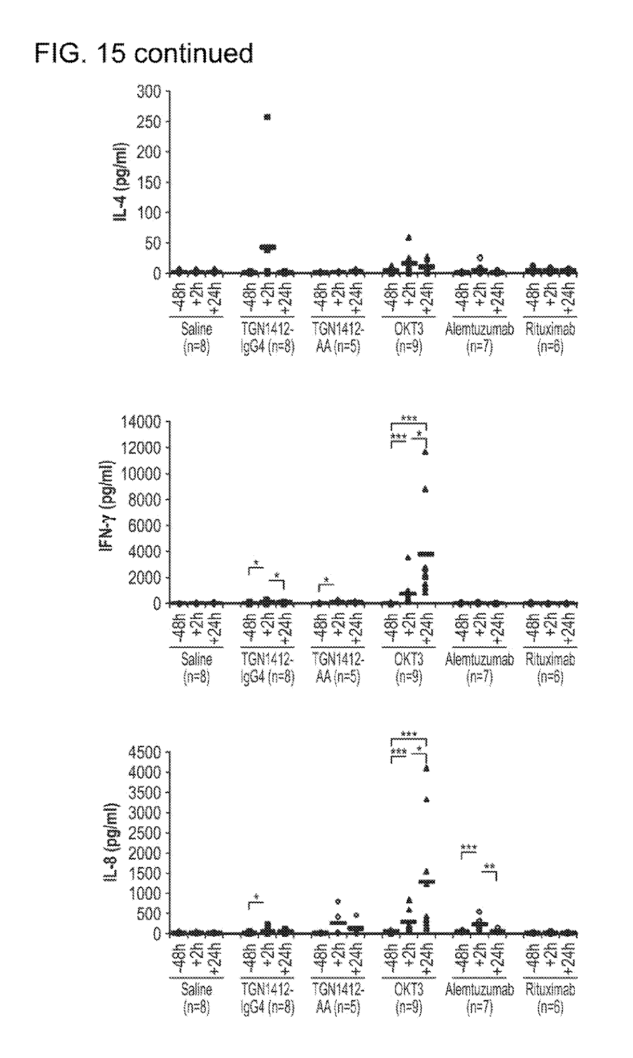

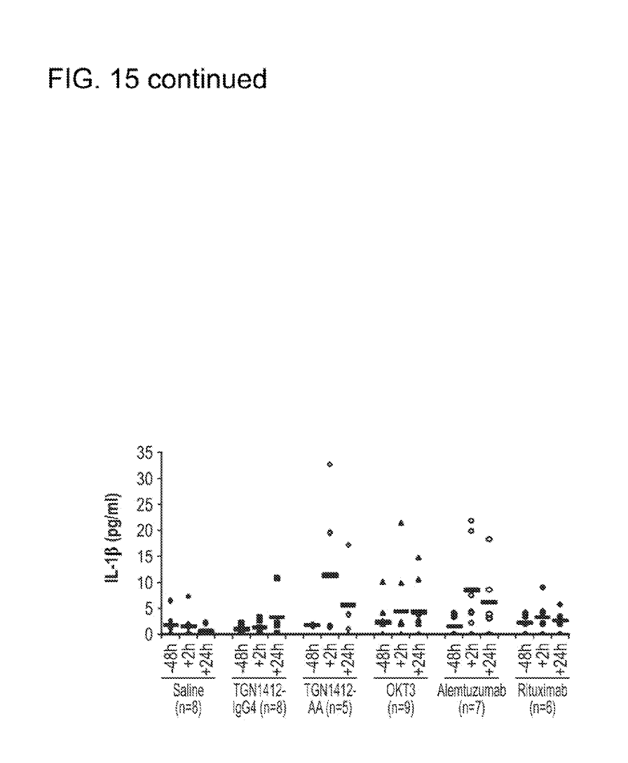

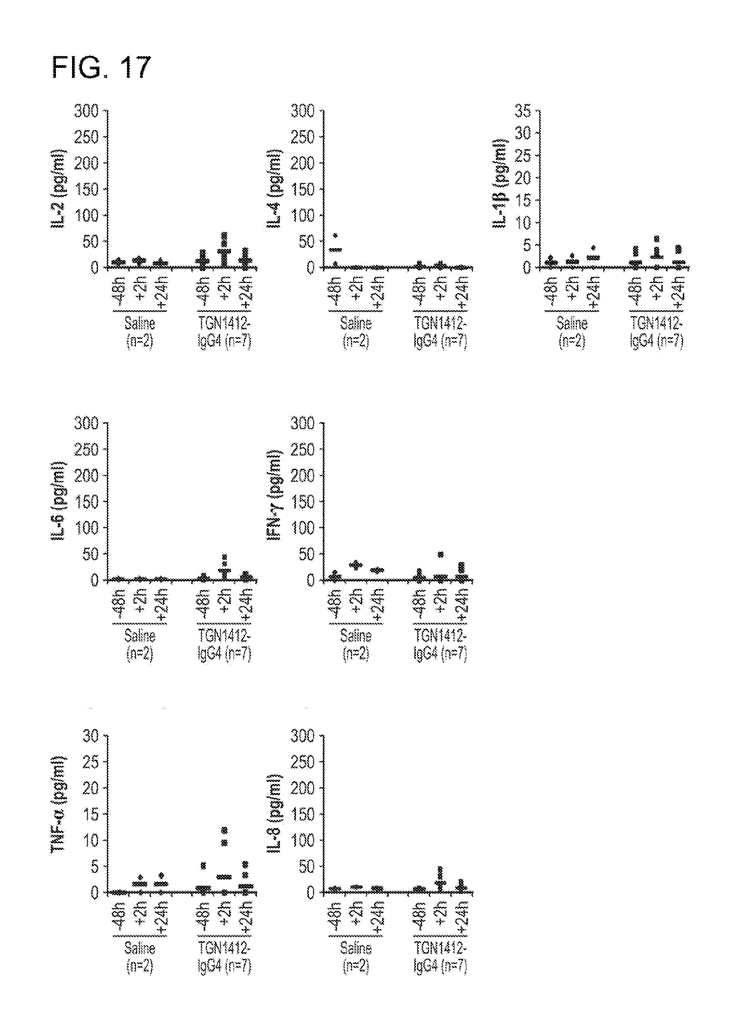

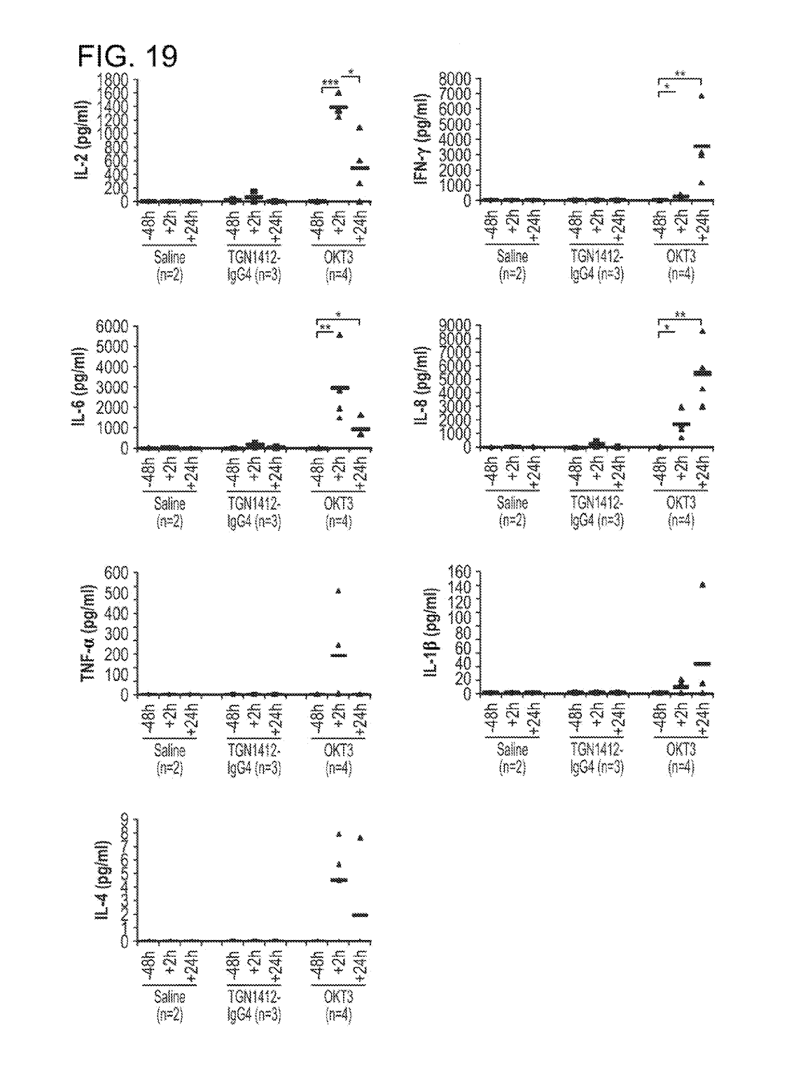

Whether the cytokine-treated humanized mouse model can predict the severe side-effect of TGN1412 antibody was tested. Humanized mice were constructed by engrafting NSG newborn pups with human HSCs. To enhance human immune responses, reconstituted mice were hydrodynamically injected with plasmids encoding the human IL-15 and Flt-3L. Seven days after the cytokine plasmid injection, the resulting cytokine-treated humanized mice were injected i.v. with 1 mg/kg of the TGN1412-IgG4 or a FcR-non-binding mutated version the TGN1412-AA. At 2 hours-post injection, mice from both treated groups showed a significant increase in serum levels of human IL-2, IFN-γ, TNF-α and IL-8, in comparison with a control (Saline) group. The IL-2 level was slightly lower in TGN1412-IgG4-treated mice as compared to the TGN1412-AA-treated mice. While IL-2 level returned after 24 hours to the pretreatment measure, the levels of IL-6, IL-8 and IFN-γ remained elevated (FIG. 15). Mice with increased levels of cytokine production showed clinical signs of weakness accompanied by a dramatic decrease in motility. However the reconstituted mice not injected with plasmids encoding the human IL-15 and Flt-3L showed no significant change in serum level of cytokines at 2 and 24 hours-post TGN1412-IgG4 injection (FIG. 17). Experiments were also conducted after human M-CSF plasmid injection to augment the number of human monocytes and macrophages with the objective to increase the cytokines release after TGN1412-IgG injection (FIG. 19). Despite the observed similarity of the amount of cytokine release between the TGN1412-IgG4 and the FcR-non-binding mutated version the TGN1412-AA, in vitro experiments have shown increase of inflammatory cytokines production after TGN1412 binding to the plastic culture plate. The extent the binding to the FcR contributes to the cytokine release as it was reported previously in an in-vitro study was analyzed, even if it was known that the IgG4 bind less efficiently to the Fc-receptor. At 2 hours-post injection, mice treated with M-CSF plasmid in comparison with IL-15 and Flt-3L treated mice showed an increase in serum levels of human IL-6 (36.8 vs 173.8 pg/ml) and IL-8 (131.8 vs 234.0 pg/ml) (FIGS. 15 and 19). These results confirmed the generation of a human cytokine storm in the cytokine-treated humanized mouse model.

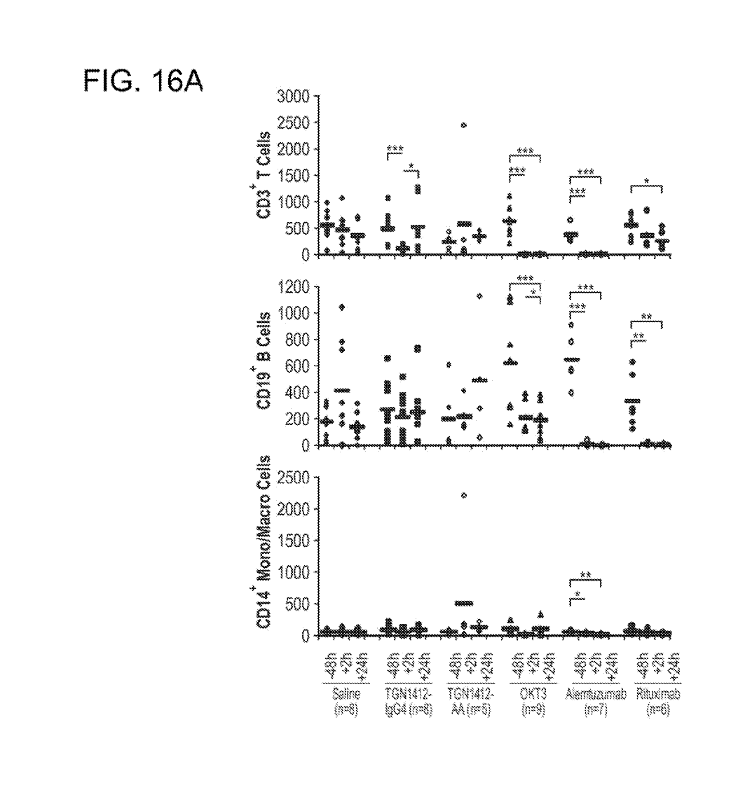

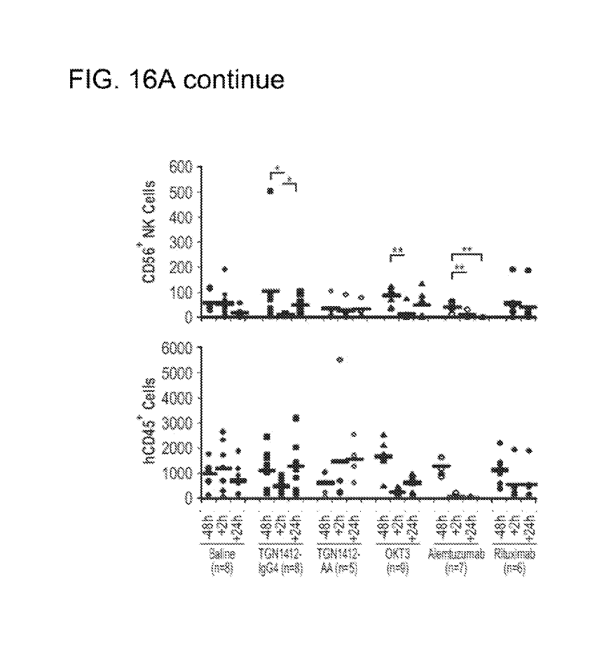

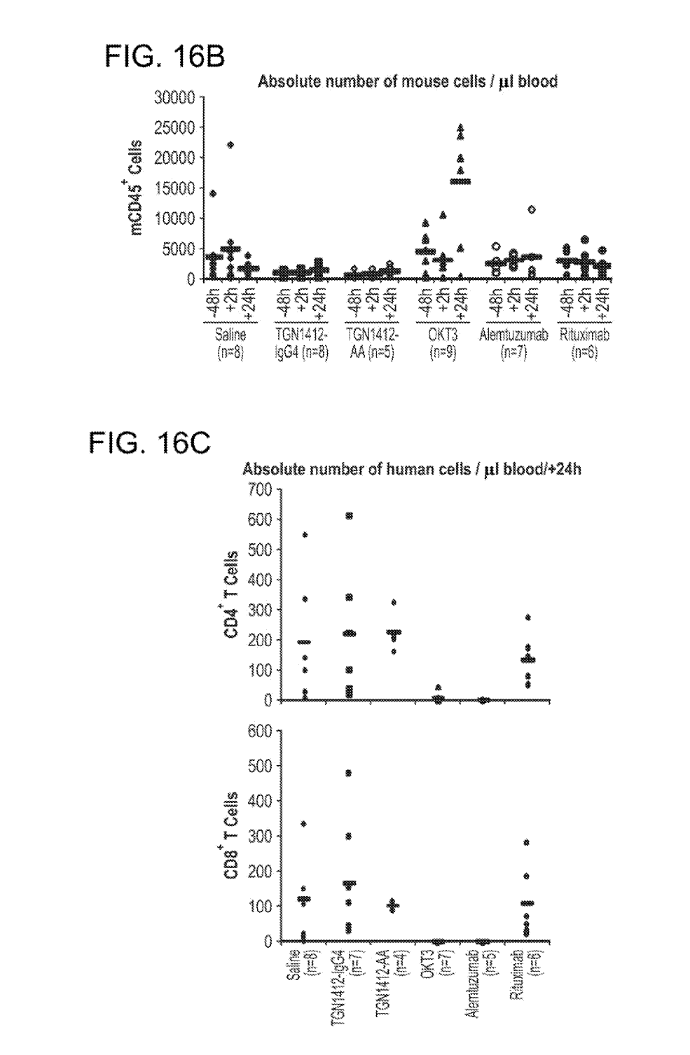

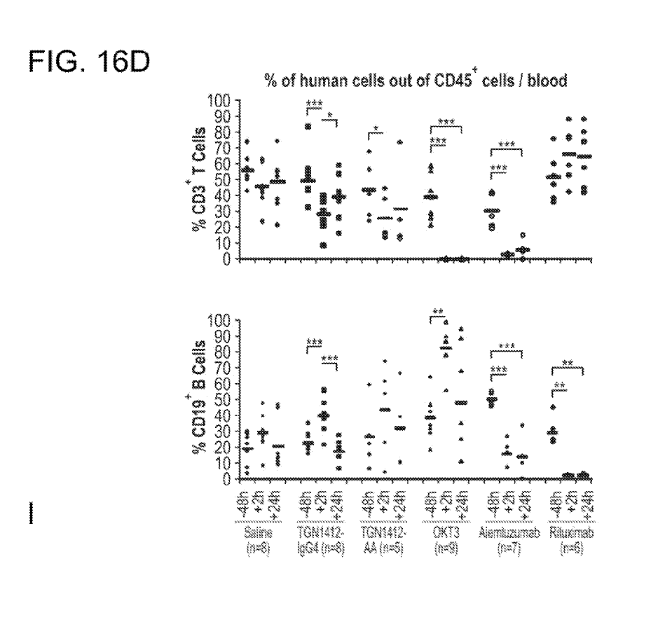

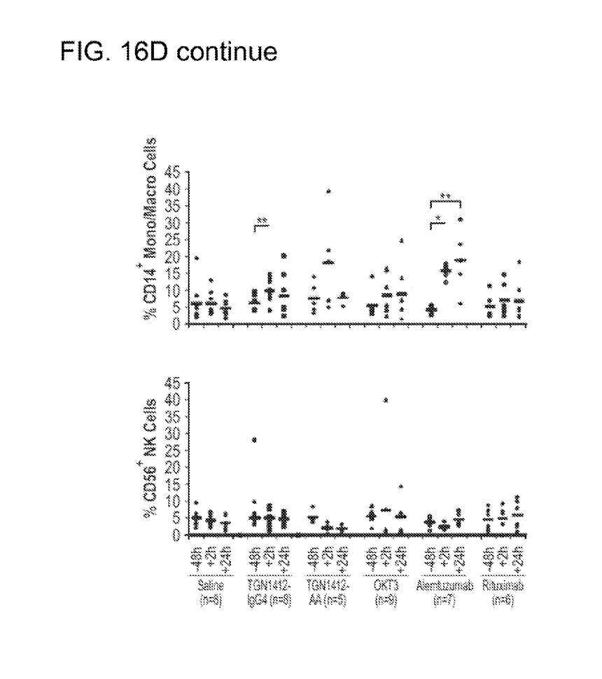

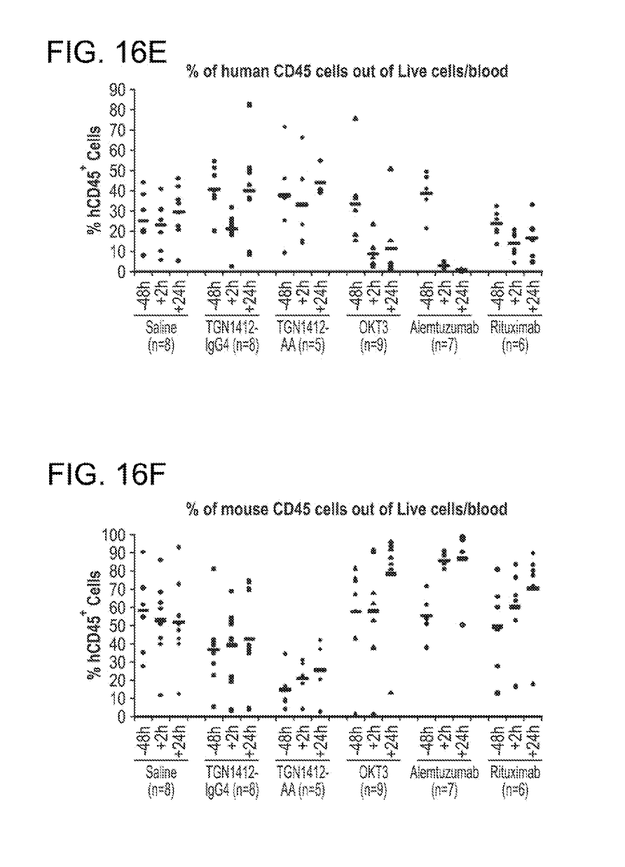

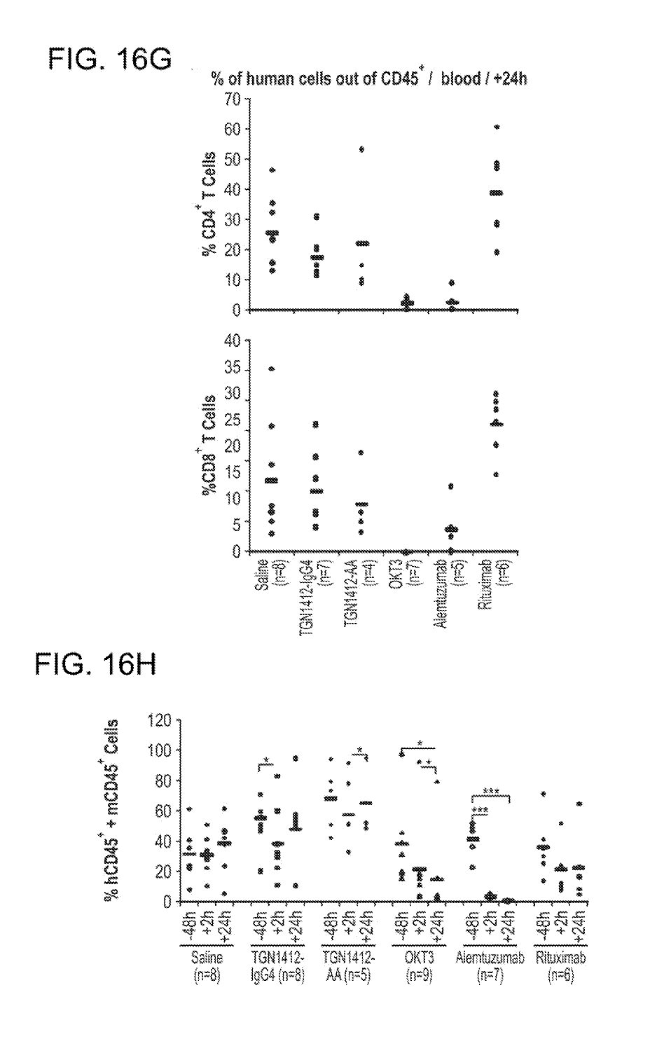

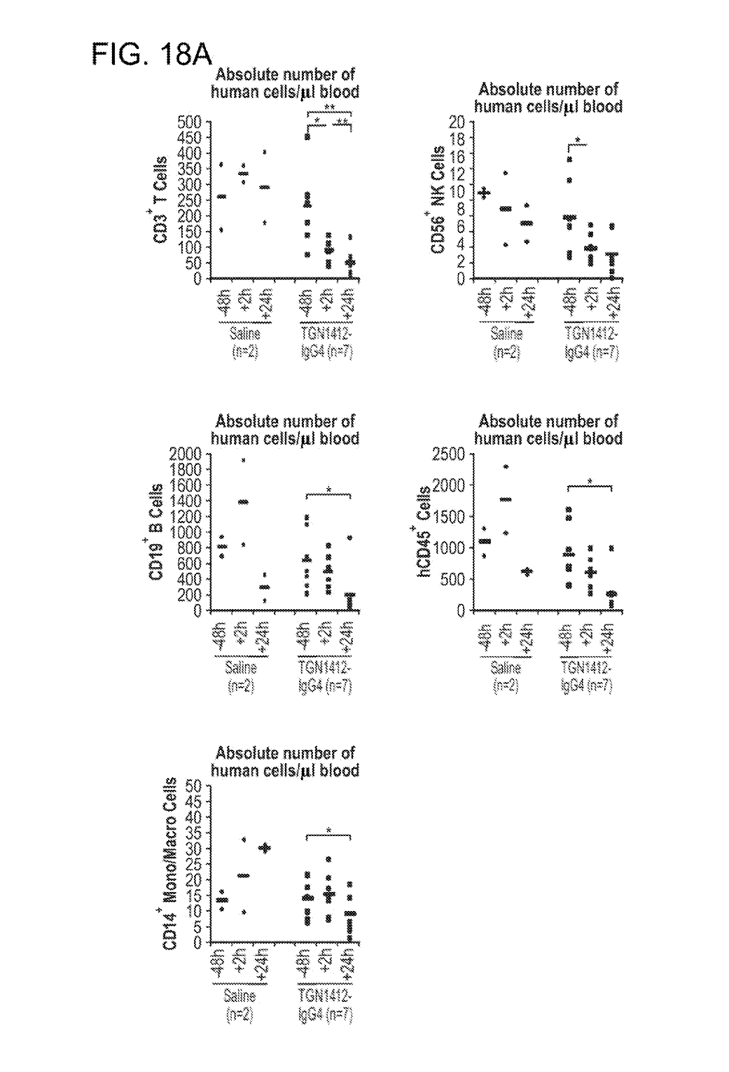

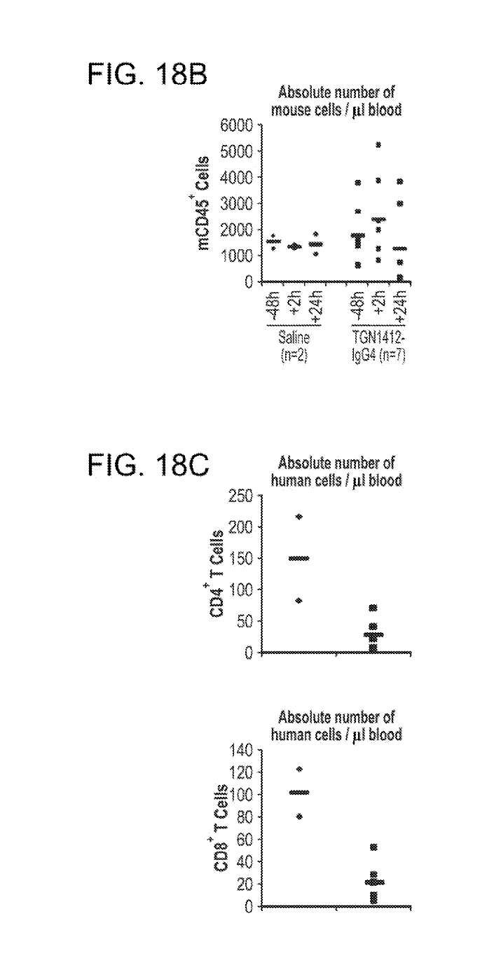

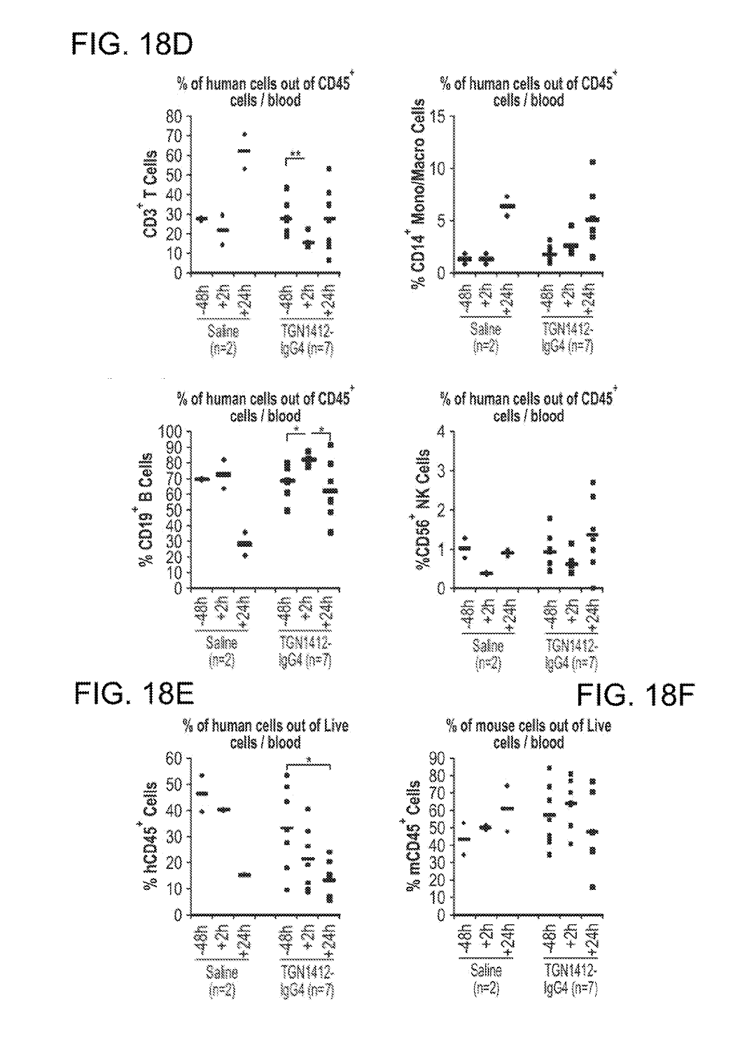

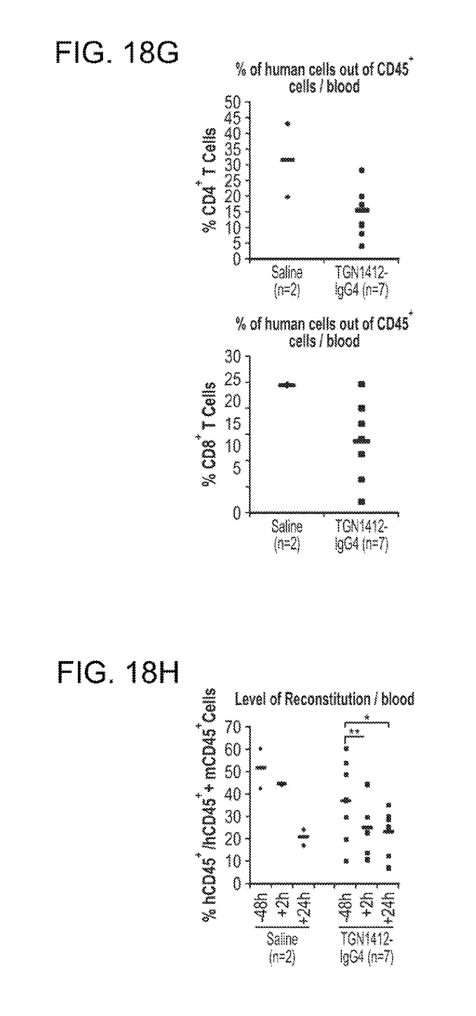

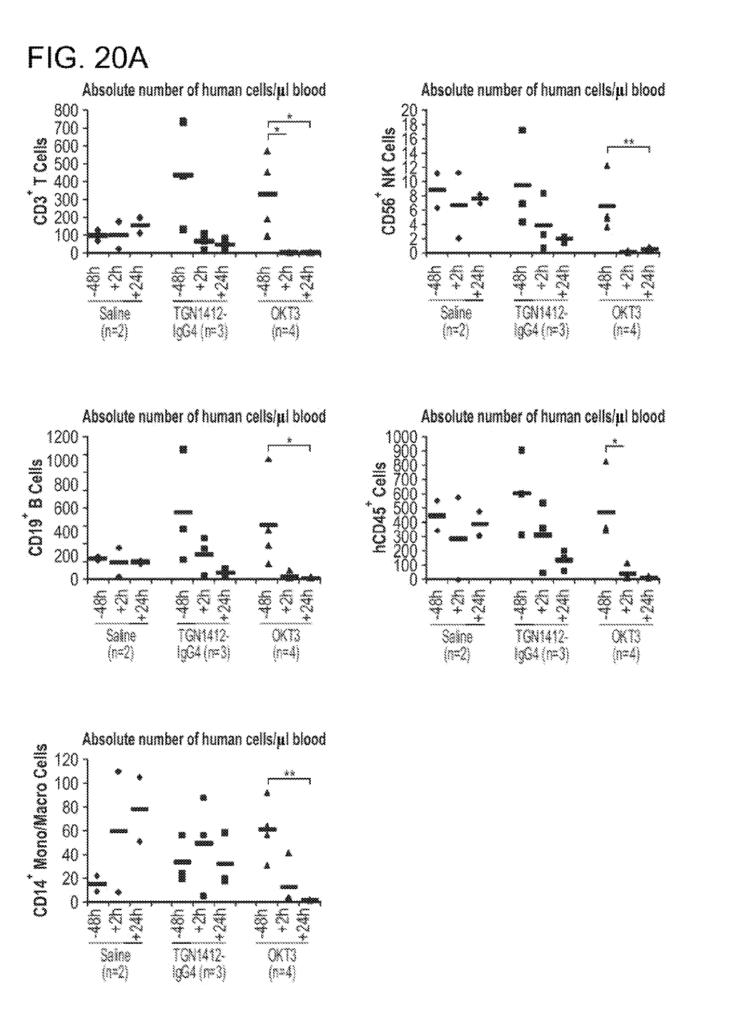

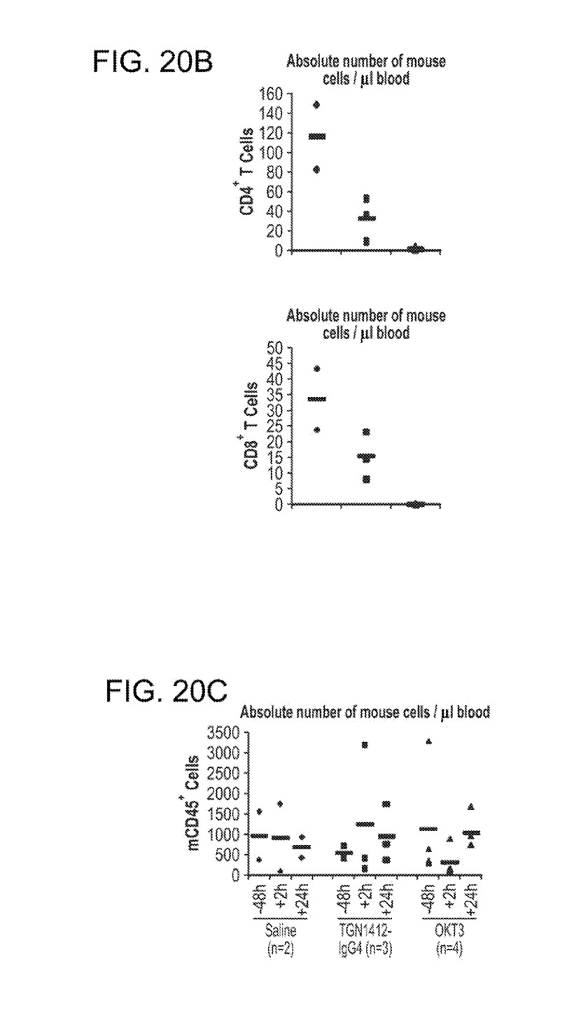

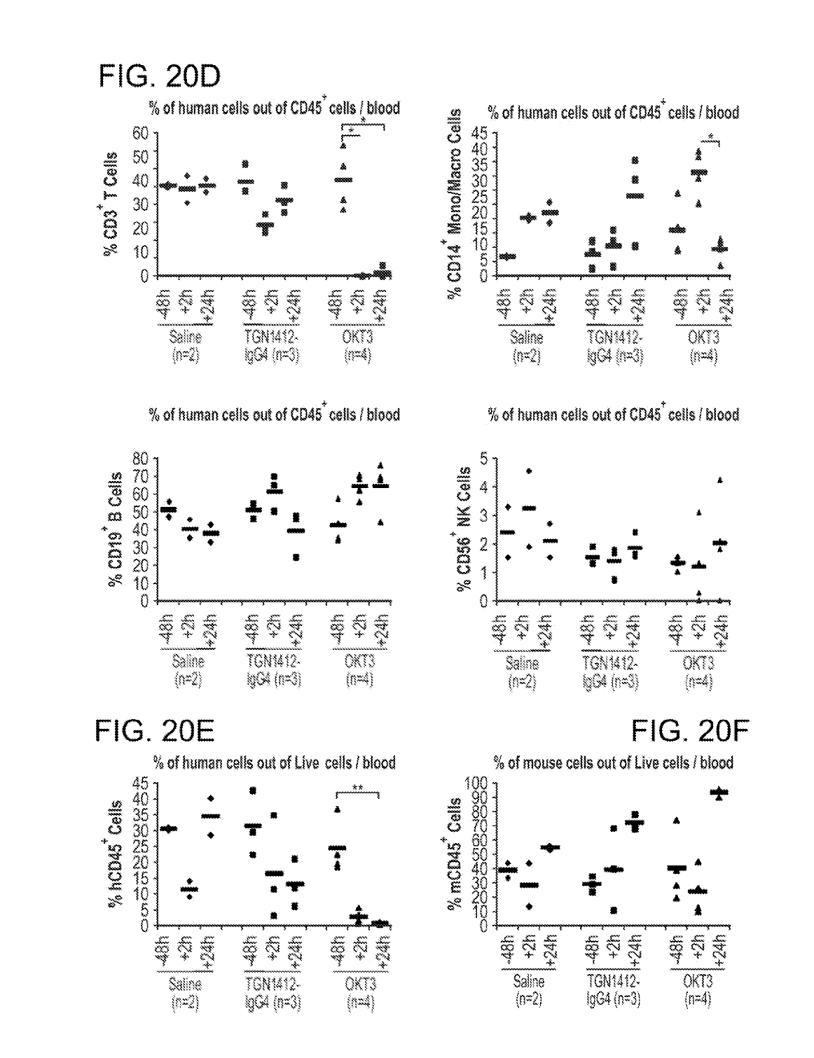

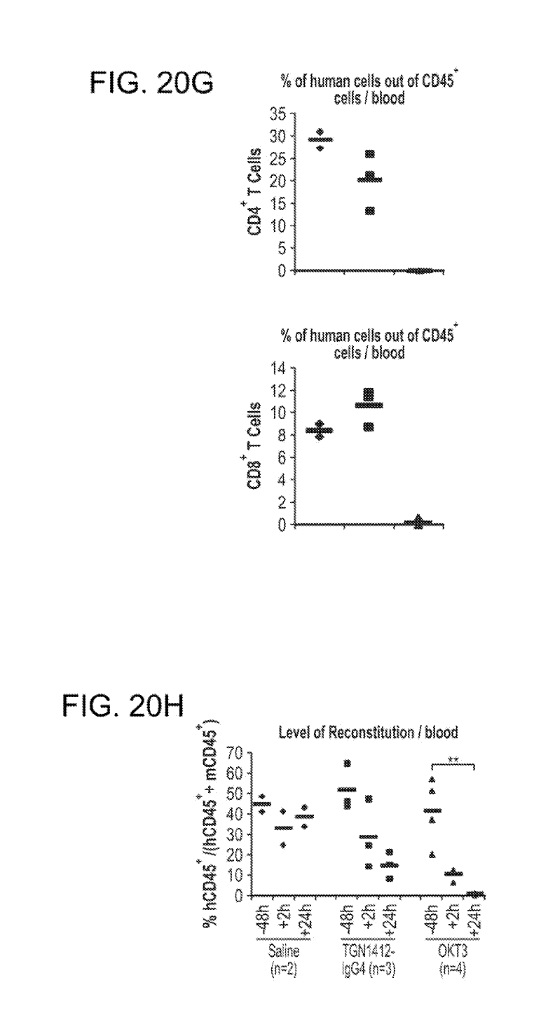

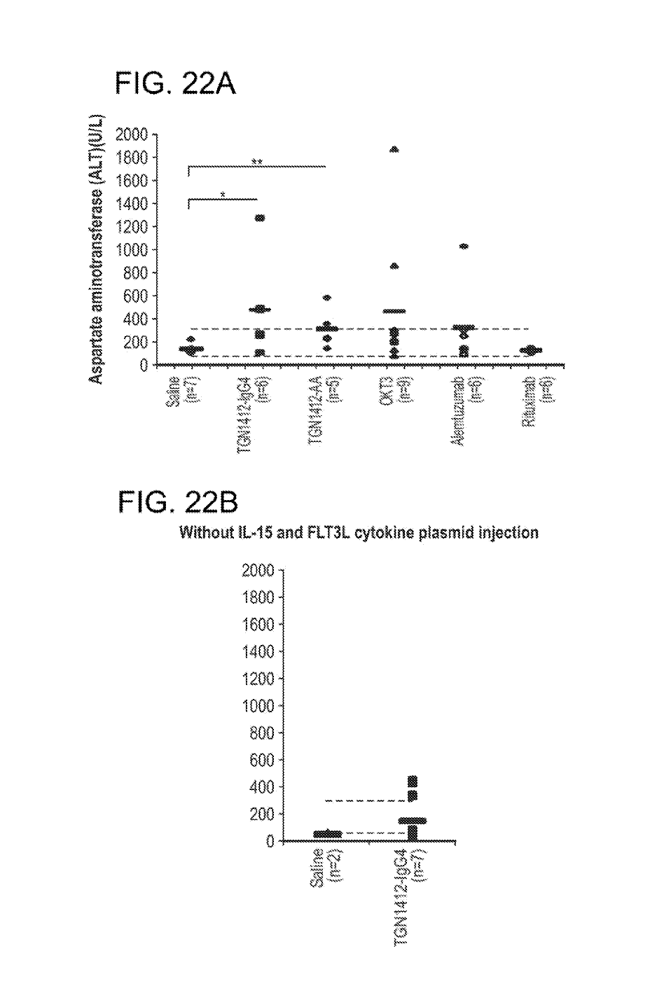

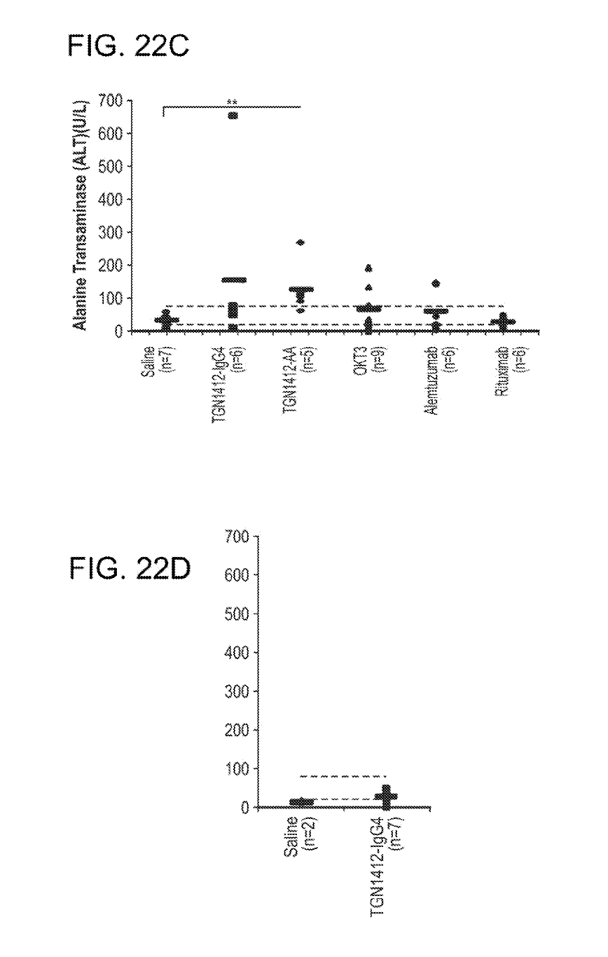

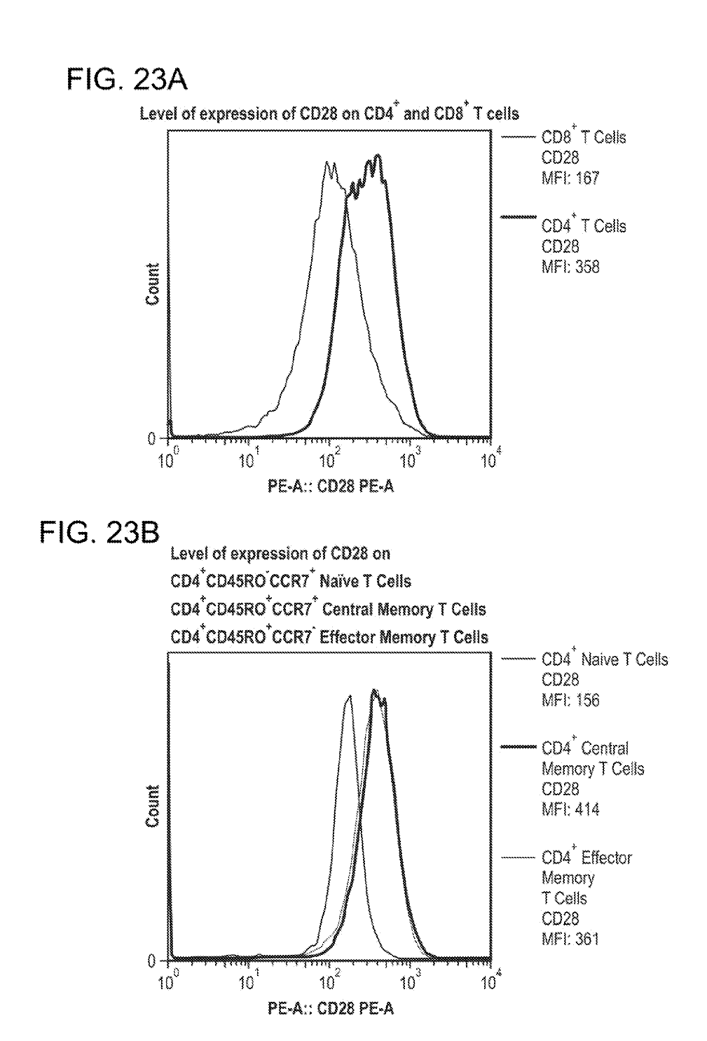

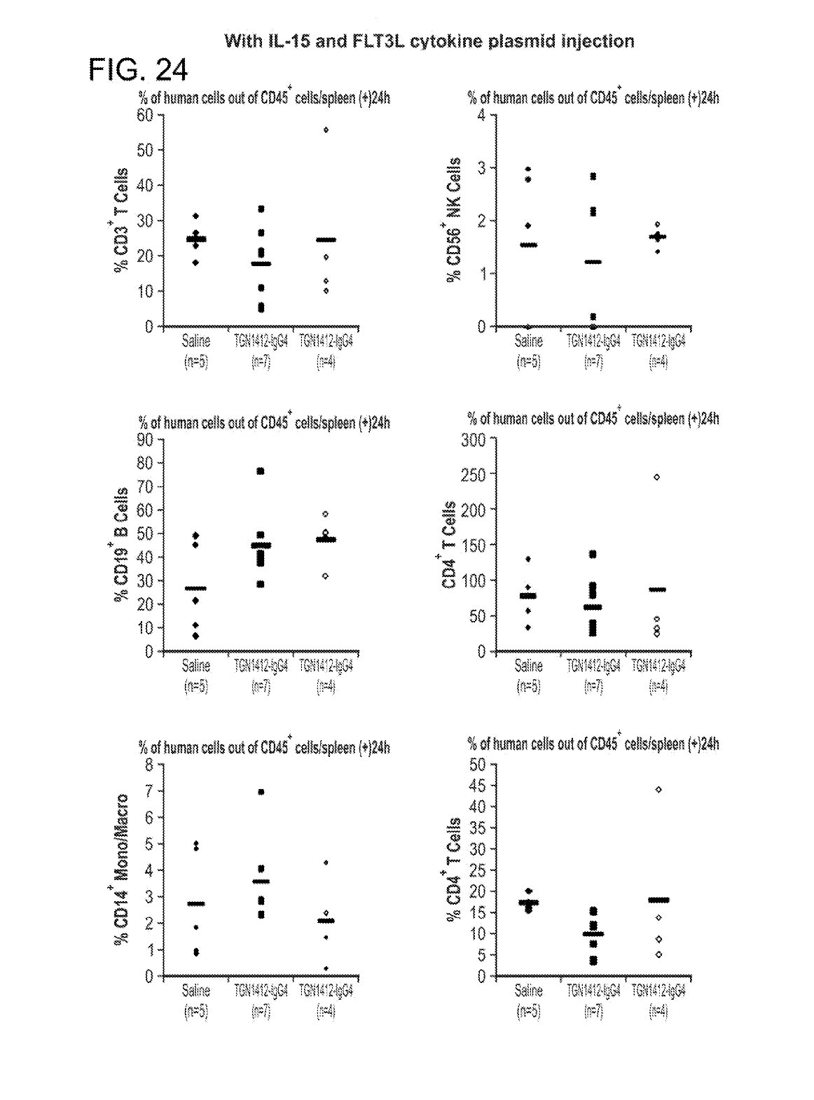

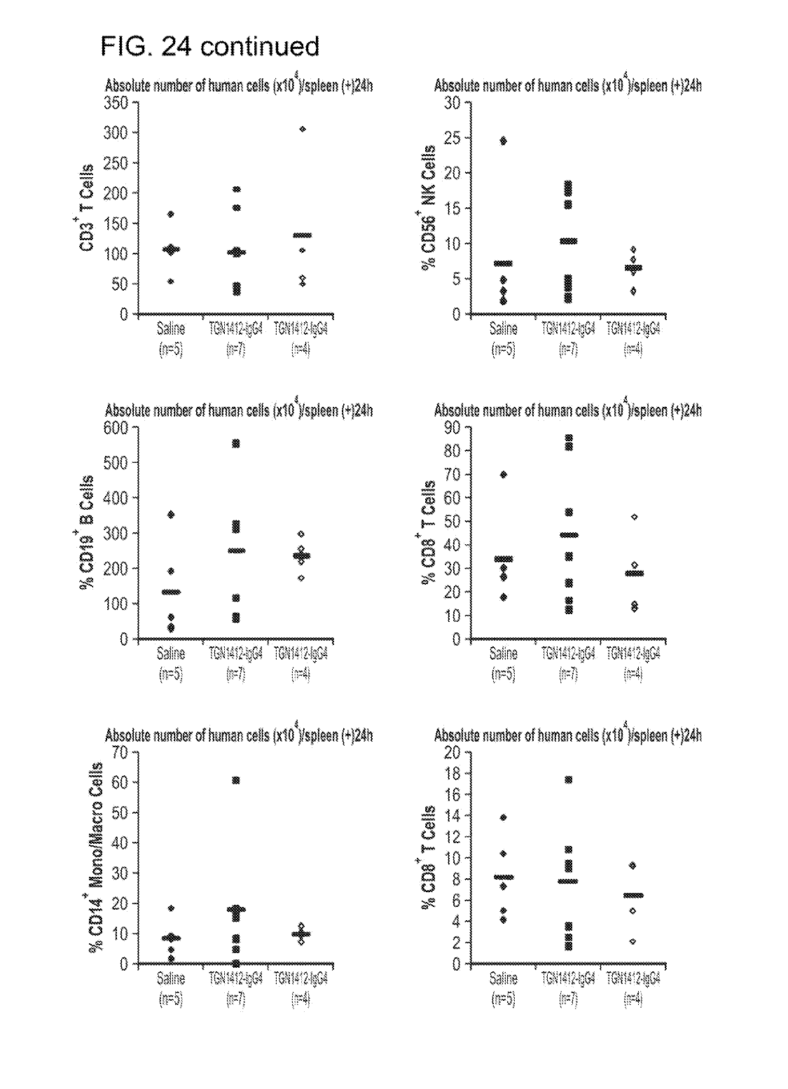

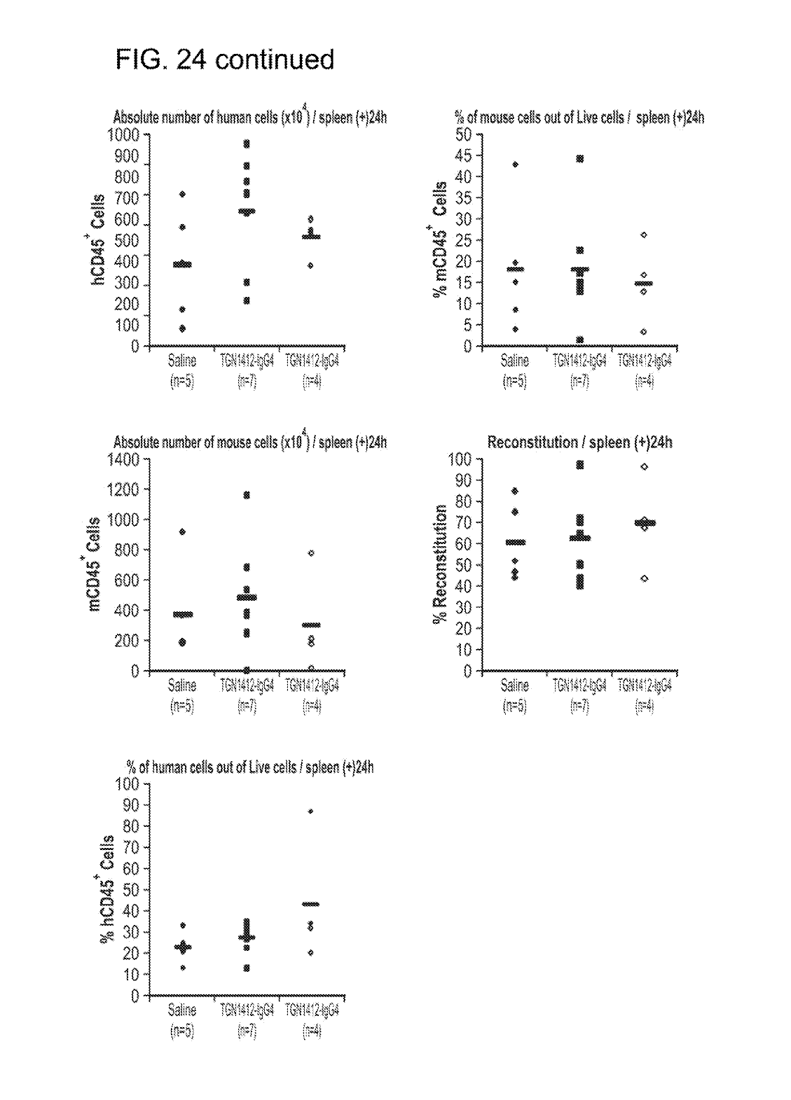

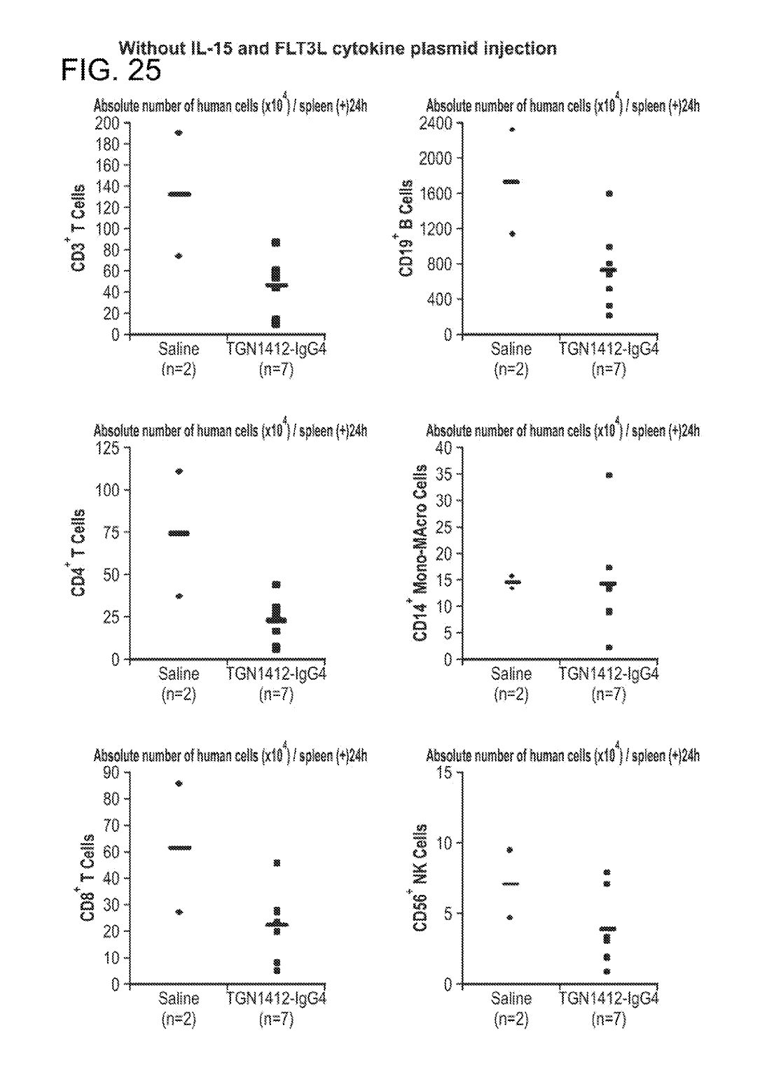

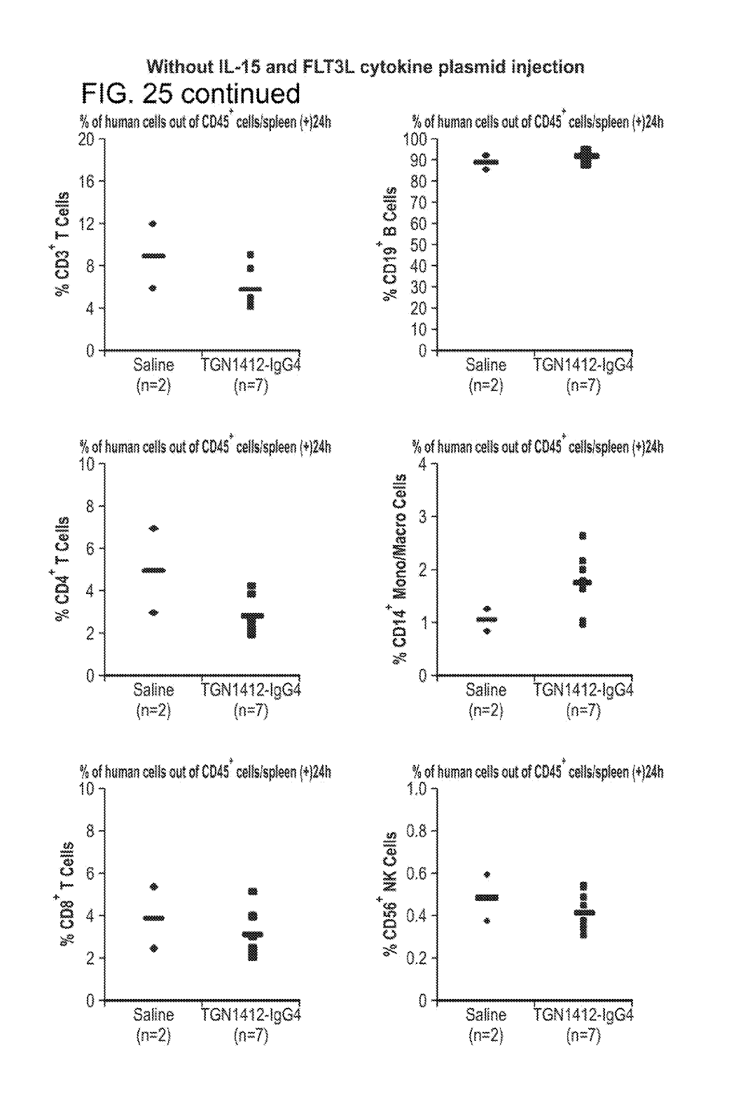

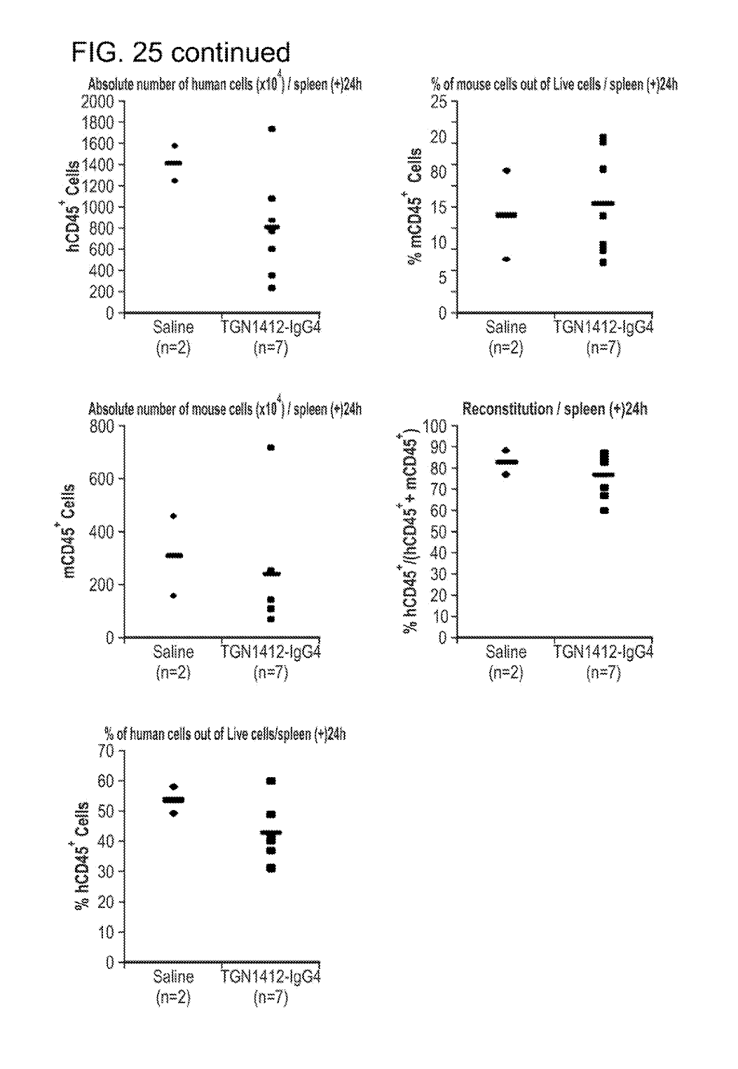

In the cytokine-treated humanized mouse, quantification of cell revealed that T cell numbers were reduced most, followed by NK cells, whereas the numbers of B cells (except for M-CSF treated mice) and monocytes remained unchanged (FIGS. 16A and 20A). At 2 hrs post-injection cytokines, mice injected with the TGN1412-IgG4 showed a more prominent decrease of circulating T cells and NK cells in the peripheral blood compared to the treatment with TGN1412-AA (FIG. 16A). Regardless of cytokines levels a comparable result was observed with the cytokine none treated humanized mouse although a diminution of the B cells absolute number was also noted 24 hrs after the TGN1412-IgG4 injection (FIGS. 18A, 18C). A similar trend was observed in the spleen of mice treated or not with plasmids encoding the human IL-15 and Flt-3L at 24 hrs post injection (FIGS. 24 and 25). The noted reduction of T cells was comparable to the drop observed in the failed clinical trial. Furthermore, a much more prominent reduction was observed within the CD3+CD4+ subset as compared to CD3+CD8+ cells. While all CD3+ cells expressed the CD28, the mean fluorescence intensity (MFI) of CD28 on CD4+ cells was 2-fold higher than on the CD8+ subset (FIGS. 23A-23B). Moreover, the expression of CD28 within the CD4+ population was 3 time higher on memory (central and effector) T cells as compared to naïve T cells (FIGS. 23A-23B). Lastly, the liver toxicity biomarkers: the level of Aspartate Aminotransferase (AST) (FIGS. 22A, 22B) and the Alanine Transaminase (ALT) were significantly elevated only in the blood of the treated groups (FIGS. 22C, 22D).

### OKT3 Induces Similar Adverse Side Effects in Cytokine-Treated Humanized Mice as in Humans

Following a similar experimental approach as used for the TGN1412 testing, the effect of OKT3 treatment of humanized mice was evaluated. Cytokine-treated humanized mice were injected with 1 mg/kg of OKT3, and blood was sampled at 2 and 24 hrs. A significant increase in the circulating levels of IL-2, IL-6, IL-8 and IFN-γ was observed as early as 2 hrs post-injection when compared the baseline level before human mAb injection (FIG. 15). While the IL-2 expression was diminished 24-hours post injection, the other cytokines remained elevated 24 hrs post injection. Notably, the cytokine concentrations were 3 to 10 fold higher in OKT3 treated mice as compared TGN1412-treated mice. At 2 hours-post injection, as was observed with TGN1412, mice treated with M-CSF plasmid in comparison with IL-15 and Flt-3L treated mice showed a increase in serum levels of human IL-6 (131.8 vs 1512.8 pg/ml) and IL-8 (266.5.8 vs 722.6 pg/ml) (FIGS. 15 and 19). Furthermore, a complete depletion of T cells was noticed at both 2 and 24 hrs post-injection (FIGS. 16A, 16D). Comparable to TGN1412 treated mice a transient decrease in the relative and absolute number of NK cells was observed after OKT3 treatment (FIGS. 16A, 16D). In contrast with TGN1412 treatment, OKT3 injection induced a 4-fold reduction of the CD14+ cells at 2 hours post-injection (FIG. 16A).

### Alemtuzumab Induces Some Human Inflammatory Cytokines but a Dramatic Depletion of Lymphocytes, NK Cells and Monocytes in Humanized Mice

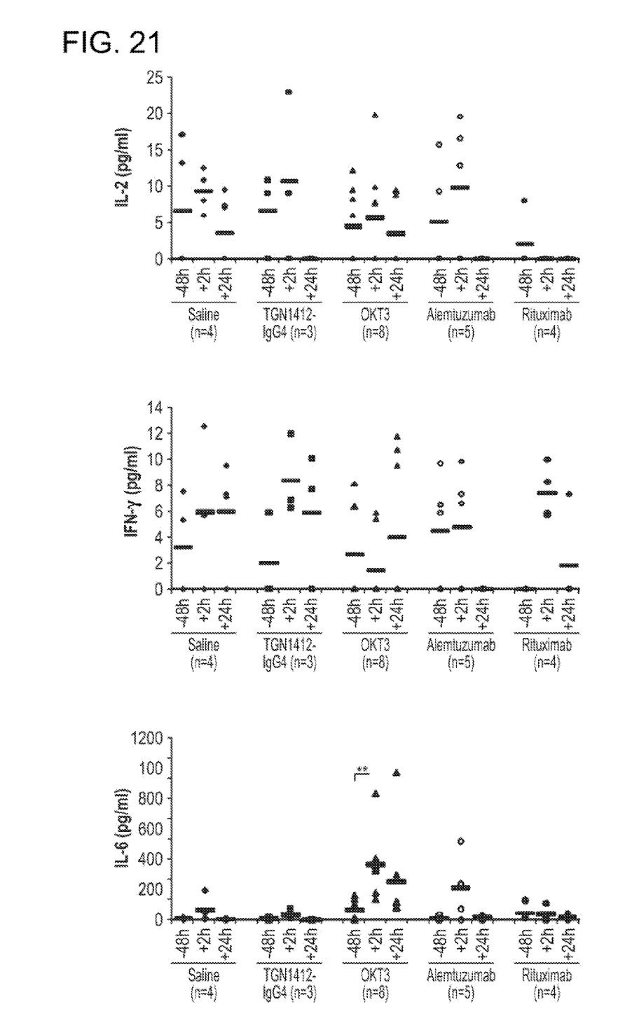

Cytokine-treated humanized mice were injected with 1 mg/kg Alemtuzumab. An increase in the serum levels of IL-2, IL-6, IL-8 and IL-1β was detected 2 hours post-injection when compared with baseline levels (FIG. 15). However, most cytokines, except IL-1β, had returned to the baseline level by 24 hrs post injection. The observation was comparable with a previous study of IL-2 (31.6 vs 14.0 pg/ml), IL-6 (19.2 vs 88 pg/ml) and IL-8 (224.1 vs 6050 pg/ml) response of PBMCs to Alemtuzumab incubated in aqueous phase. Moreover, a complete depletion of lymphocytes, monocytes and NK cells was observed after injection of CD52 (FIGS. 16A, 16B). An increase of mouse IL-6 cytokine was noted for cytokine-treated humanized mice injected with Alemtuzumab and OKT3 most probably related to the binding to the Fc-receptor on mouse monocytes and macrophages (FIG. 21).

Rituximab does not Induce any Significant Side Effects

Cytokine-treated humanized mice were injected i.v. with Rituximab (1 mg/kg) and were compared to a saline-injected control group. The cytokine expression measured after the Rituximab treatment differs dramatically from the response seen with both the TNG1412 and OKT3, as no release of any of the measured cytokines (IL-1β, IL-2, IL-4, IL-6, IL-8, IFN-γ, TNF-α) was noted (FIG. 15). Furthermore, injected mice didn't show any signs of weakness or distress. As expected, the cell type affected, among the different cell lineages, was the B cell (percentage and total number) as most cells where depleted at 2 hours post-injection (FIGS. 16A, 16B). Although a reduction of the T cells number 2 and 24 hrs after mAb injection was also observed in accordance with the result of a clinical trial describing a transient decrease of the peripheral T Cells counts post Rituximab infusion (Protocol number: WA17042). In addition, the liver enzymes levels (AST and ALT) remained unchanged after the monoclonal antibody treatment (FIGS. 22A-22D).

### DISCUSSION

The suspension of clinical trials due to major side-effects reflects the failure of conventional pre-clinical systems to accurately predict such adverse responses in humans. As described herein, whether the NSG mice engrafted with human HSCs and treated with human cytokines to enhance the immune system is able to serve as an accurate and reliable system for assaying and predicting immune toxicity in humans was investigated. Four monoclonal antibodies specific for CD28, CD3, CD52 and CD20 were used to validate the system because these antibodies are known to exhibit different side effects in humans. A major side effect is “cytokine storm”; the cytokine release in treated mice was measured and the secretion profiles were compared to the clinical data. Following TGN1412 injection, mice showed a significant increase in serum levels of human IL-2, IFN-γ, TNF-α and IL-8. The kinetics of cytokine production was in line with the expression curve depicted in the clinical trial, where all 6 volunteers manifested a similar trend. However, only 2 of the 6 recruits showed an increased level of IL-4 and 3/6 for IL-1β. Similarly, only 2 of 9 mice showed an increased IL-4 level but no change in the IL-1β level. Besides the similarities observed at the cytokine level, T cell lymphopenia dramatically decreased in both humans and humanized mice, and CD4+ cells seemed to be more profoundly affected probably because they showed a higher expression of the CD28. The single difference noted was the drop of the monocytes counts only in the clinical trial; this could be related to an initial difference in the monocytes numbers between the mice and humans. It is noteworthy to mention that none of these symptoms is detected in the initial study in wild type mice prior to the clinical trial. In the cytokine-treated humanized mice the CD4 effector memory cells CD4+CD45RO+CCR7-CD28+ were identified as responsible for the production of pro-inflammatory cytokines following TGN1412 stimulation. This subset is specific to humans and absent in non-humans primates, and wild type mice, and can account for the absence of significant side effects detected in the conventional models. Collectively, the results achieved in the model described herein, if performed at the right time of the drug development process, would have alerted the hazard of using such molecule in human testing, and therefore prevented the catastrophic event seen in the failed clinical trial.

Similar to the results obtained with TGN1412, the administration of OKT3 in the cytokine-treated humanized mice resulted in elevation of human IL-2, IL-6, IL-8 and IFN-γ cytokines. These findings were in parallel with results reported in kidney transplanted patients, treated with OKT314.

The T cell depletion noted was also in accordance with the results in patients, were lymphopenia and neutropenia as evident at two hours after the injection. Alemtuzumab, known to induce a cytokine storm in patients, was also tested. The administration of Alemtuzumab to the cytokine-treated humanized mice induced an elevated level of human IL-2, IL-6, IL-8 and IL-1β at 2 hrs but returned to the baseline level at 24 h, except for IL-1β. A severe reduction in B and T cells, NK cells and monocytes was observed 2 hrs after the injection, with only residual monocytes cell remaining at 24 hrs. All these results were similar with what was observed in patients treated with Alemtuzumab.

Rituximab, a monoclonal antibody known for having no to minimal inflammatory cytokines release in patients, in contrast with the severe adverse effects described for the TGN1412 and OKT3, was also tested. In one clinical study, it was shown that following Rituximab administration, some leukemic patients showed a slight rise of the IL-6 (˜80 pg/ml) and TNF-β (˜870 pg/ml) levels with minimal change in IL-2, IL-4 and IFN-γ compared to the baseline levels. The cytokine release was transient as both cytokine levels returned to baseline after completion of the initial Rituximab infusion. This induced expression was related to leukemic patients with large numbers of B lymphocytes. The absence of cytokines release in the study described herein could be related to the low number of CD20+ lymphocytes in comparison with the chronic lymphocytic leukemia patients. However, the model described herein is more comparable to the outcome noted in the rheumatoid arthritis patients, where only a depletion of peripheral-blood B cells was noted. It might be of interest in testing Rituximab in humanized mice in the presence of its target human tumor cells. T cells are known as the principals' producer of inflammatory cytokines after injection of OKT3 and TGN1412, although for Rituximab and Alemtuzumab which have no intrinsic T cell-activation potential, they can be responsible for clinically relevant cytokine release most probably through FcR binding on other cytokines producers like monocytes, macrophages and NK cells.

Finally, strong evidence showing that the cytokine-treated humanized mice present a robust prediction tools for drug immunotoxicity testing is presented herein. This system contributes to estimate and sets up the first-dose-in-man, based on the ‘no observed adverse effect level’ (NOAEL) and on the ‘minimal anticipated biological effect level’ (MABEL) as determined in toxicity studies. Furthermore, another application would be to identify the different mechanisms that underlie the side effect and determine the most sensitive, and predictive common biological markers to use for the cytokine release syndrome in humans.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of determining whether an agent causes immune toxicity in a human comprising:
a) administering the agent to a non-human mammal that has been engrafted with human hematopoietic stem cells (HSCs) and administered one or more human cytokines; and
b) b) determining whether the agent causes immune toxicity in the non-human mammal,
wherein if the agent causes immune toxicity in the non-human mammal then the agent causes toxicity in a human.

2. The method of claim 1 wherein the non-human mammal is a mouse.

3. The method of claim 2 wherein the mouse is an immunodeficient mouse.

4. The method of claim 3 wherein the immunodeficient mouse's immune system is populated with human T cells.

5. The method of claim 2, wherein the immunodeficient mouse is a NOD.Cg-Prkdcscid 112rgtm1Wjl/SzJ (NOD Scid gamma) mouse.

6. The method of claim 1 wherein the one or more human cytokines comprise interleukin-15 (IL-15), IL-4, Fms-related tyrosine kinase 3 ligand (Flt-3L), granulocyte/macrophage colony stimulating factor (GM-CSF), macrophage colony stimulating factor (MCSF), stem cell growth factor, IL-3 or a combination thereof.

7. The method of claim 1 wherein the non-human mammal is treated with two cytokines.

8. The method of claim 7 wherein the cytokines are IL-15 and Flt-3L.

9. The method of claim 1 wherein the agent is a therapeutic agent.

10. The method of claim 9 wherein the therapeutic agent is an antibody, a protein, a nucleic acid, a polysaccharide, a lipopolysaccharide, a lipoprotein, a lipid, a microbial antigen or a nanoparticle.

11. The method of claim 10 wherein the antibody is a monoclonal antibody.

12. The method of claim 1 wherein whether the agent causes immune toxicity in the non-human mammal is determined by measuring immune cell phenotype, increased expression of one or more liver enzymes, increased expression of one or more pro-inflammatory cytokines or a combination thereof that occurs in the non-human mammal after administration of the agent.

13. The method of claim 12 wherein the immune cell phenotype is measured by determining cell surface markers, proliferation, activation or a combination thereof of one or more immune cells.

14. The method of claim 13 wherein the one or more immune cells comprise T cells, B cells, natural killer (NK) cells, monocytes/macrophages, CD45.1+ cells or a combination thereof.

15. The method of claim 14 wherein T cells are determined by measuring cells expressing CD3+, B cells are determined by measuring cells expressing CD19+, NK cells are determined by measuring cells expressing CD56+, and monocytes/macrophages are determined by measuring cells expressing CD14+.

16. The method of claim 12 wherein the one or more pro-inflammatory human cytokines comprise interleukin-2 (IL)-2, IL-6, IL-8, IL-1β, IL-4, gamma interferon (IFN-γ), tumor necrosis factor alpha (TNF-α) or a combination thereof.

17. The method of claim 16 wherein the one or more pro-inflammatory cytokines are measured using fluorescence activated cell sorting.

18. The method of claim 12 wherein the one or more liver enzymes comprises aspartate, alanine aminotransferase or a combination thereof.

19. The method of claim 1 wherein whether the agent causes toxicity in the non-human mammal is determined within one or more hours, days, weeks, months or years after the agent is administered.

20. The method of claim 1 further comprising comparing whether the agent causes toxicity in a control.

21. A method of determining whether administration of an agent to a human will cause cytokine release syndrome in the human comprising:
a) administering the agent to a non-human mammal that has been engrafted with human hematopoietic stem cells (HSCs) and administered one or more human cytokines; and
b) determining whether the agent causes cytokine release syndrome in the non-human mammal,
wherein if the agent causes cytokine release syndrome in the non-human mammal then the agent will cause cytokine release syndrome in the human.

22. The method of claim 21 wherein the non-human mammal is a mouse.

23. The method of claim 22 wherein the mouse is an immunodeficient mouse.

24. The method of claim 23 wherein the immunodeficient mouse's immune system is populated with human T cells.

25. The method of claim 23 wherein the immunodeficient mouse is a NOD.Cg-Prkdcscid 112rgtm1Wjl/SzJ (NOD Scid gamma) mouse.

26. The method of any one of claim 21 wherein the one or more human cytokines comprise interleukin-15 (IL-15), IL-4, Fms-related tyrosine kinase 3 ligand (Flt-3L), granulocyte/macrophage colony stimulating factor (GM-CSF), macrophage colony stimulating factor (MCSF), stem cell growth factor, IL-3 or a combination thereof.

27. The method of any one of claim 21 wherein the non-human mammal is treated with two cytokines.

28. The method of claim 27 wherein the cytokines are IL-15 and Flt-3L.

29. The method of any one of claim 21 wherein the agent is a therapeutic agent.

30. The method of claim 29 wherein the therapeutic agent is an antibody, a protein, a nucleic acid, a polysaccharide, a lipopolysaccharide, a lipoprotein, a lipid, a microbial antigen or a nanoparticle.

31. The method of claim 30 wherein the antibody is a monoclonal antibody.

32. The method of any one of claim 21 wherein whether the agent causes cytokine release syndrome in the non-human mammal is determined by measuring immune cell phenotype, increased expression of one or more liver enzymes, increased expression of one or more pro-inflammatory cytokines or a combination thereof that occurs in the non-human mammal after administration of the agent.

33. The method of claim 32 wherein the immune cell phenotype is measured by determining cell surface markers, proliferation, activation or a combination thereof of one or more immune cells.

34. The method of claim 33 wherein the one or more immune cells comprise T cells, B cells, natural killer (NK) cells, monocytes/macrophages, CD45.1+ cells or a combination thereof.

35. The method of claim 34 wherein T cells are determined by measuring cells expressing CD3+, B cells are determined by measuring cells expressing CD19+, NK cells are determined by measuring cells expressing CD56+, and monocytes/macrophages are determined by measuring cells expressing CD14+.

36. The method of claim 32 wherein the one or more pro-inflammatory human cytokines comprise interleukin-2 (IL)-2, IL-6, IL-8, IL-1b, IL-4, gamma interferon (IFN-γ), tumor necrosis factor alpha (TNF-α) or a combination thereof.

37. The method of claim 36 wherein the one or more pro-inflammatory cytokines are measured using fluorescence activated cell sorting.

38. The method of claim 32 wherein the one or more liver enzymes comprises aspartate, alanine aminotransferase or a combination thereof.

39. The method of claim 21 wherein whether the agent causes toxicity in the non-human mammal is determined within one or more hours, days, weeks, months or years after the agent is administered.

40. The method claim 21 further comprising comparing whether the agent causes toxicity in a control.
